(19) United States
(12) Patent Application Publication
(10) Pub. No.: US 2026/0345800 A1
(43) Pub. Date: Nov. 5, 2026

(54) OCULAR DELIVERY OF THERAPEUTIC AGENTS

(71) Applicant: ENTRADA THERAPEUTICS, INC., Boston, MA (US)

(72) Inventors: Ziqing QIAN, Lexington, MA (US); Haoming LIU, Canton, MA (US); Xiang LI, Belmont, MA (US); Patrick DOUGHERTY, Independence, OH (US); Pingjuan LI, Franklin, MA (US); Silvia PASINI, Cambridge, MA (US); Xiulong SHEN, Waltham, MA (US)

(21) Appl. No.: 19/115,637

(22) PCT Filed: Sep. 29, 2023
(86) PCT No.: PCT/US2023/034113

§ 371 Date: Mar. 26, 2025

Related U.S. Application Data
(60) Provisional application No. 63/411,840, filed on Sep. 30, 2022.
(60) Provisional application No. 63/425,807, filed on Nov. 16, 2022.

(51) Int. Cl. A61K 47/64; A61K 9/00; A61P 27/02; C12N 15/113

(52) U.S. Cl. CPC A61K 47/64; A61K 9/0048; A61P 27/02; C12N 15/113; C12N 2310/11

(57) ABSTRACT

The present disclosure is directed to compounds and compositions that contain cell penetrating peptides and therapeutic moieties for treating a disease of the eye. The compounds and compositions may be delivered to the eye for treating a disease of the eye. Method of administering the compounds and compositions to the eye for treating a disease of the eye are also provided.

### CROSS-REFERENCE TO RELATED APPLICATION

This application claims the benefit of U.S. Provisional Patent Application No. 63/411,840, filed Sep. 30, 2022, and U.S. Provisional Patent Application No. 63/425,807, filed Nov. 16, 2022, each of which is incorporated herein by reference in its entirety.

### INTRODUCTION

A number of therapeutic agents for treating ocular diseases exist or have been proposed. Some ocular therapeutic agents, such as oligonucleotides, polypeptides, or intracellularly acting small molecule therapeutics, may have limited effectiveness unless they are delivered to the correct eye location, cell-type, and/or to an intracellular compartment where their effects may be realized.

The anatomy and physiology of the eye presents challenges to delivering therapeutic agents to a desired eye location and/or specific cells of the eye. The eye includes static and dynamic barriers that tend to exclude entry of xenobiotics and discourage active absorption of therapeutic agents (see, for example, Gote, et al., J Pharm Exp Ther, September 2019, 370(3): 602-624). Static barriers include tissue that provides a passive physical barrier to therapeutic agent penetration (see Rowe-Rendleman, et al., Invest Opthamol Vis Sci, 2014 April, 55(4):2714-2730). Dynamic barriers include physiological clearance mechanisms such as aqueous turnover, vitreous flow, ocular drug metabolism, and eye metabolizing enzymes (see, id).

For different routes of administration, the eye presents distinct barriers for achieving effective levels of therapeutic agents. For example, barriers for intraocular administration, such as intravitreal injection, includes static barriers such as the vitreous humor and dynamic barriers such as anterior aqueous humor flow, posterior trans-retinal flow, and efflux transporters (see, id). Barriers of suprachoroidal administration, such as via suprachoroidal injection, includes static barriers, such as the choroid, Bruch's membrane, PRE tight junctions, and the retina, as well as dynamic barriers such as choroidal circulation and retinal circulation (see, id). Barriers for periocular administration, such as sub-tenon's injection, includes static barriers, such as the sclera, the choroid, Bruch's membrane, RPE tight junctions, the retina, as well as dynamic barriers such as subconjunctival-episcleral lymph and blood vessels, choroidal circulation, and retinal circulation (see, id). Barriers for topical administration, such as eye drops, includes static barriers, such as the cornea, the conjunctiva, the sclera, the choroid, Bruch's membrane, RPE tight junctions, and the retina, as well as dynamic barriers, such as tears and lacrimal drainage, subconjunctival-episcleral lymph and blood vessels, choroidal circulation, and retinal circulation (see, id).

To achieve a therapeutic effect, a therapeutic agent not only needs to be delivered to an appropriate location of the eye, but also needs to be delivered to the appropriate cells and cellular compartments of the eye. Carrier systems, such as polymers, cationic liposomes, or chemical modifications, for example, by the covalent attachment of cholesterol molecules, have been used to facilitate intracellular delivery. Still, intracellular delivery efficiency by these approaches is often low and improved delivery systems to increase efficacy of intracellular delivery have remained elusive.

In the late 1980s, it was discovered that the highly positively charged HIV Tat peptide could translocate across the mammalian cell membrane. Subsequently, other “cell penetrating peptides” (CPPs) have been discovered that are capable of penetrating the cell membrane at low micromolar concentrations without causing significant membrane damage. However, effective cytosolic delivery by many of these CPPs is limited by poor endosomal escape efficiency, and some may have undesirably high toxicity levels.

Accordingly, new cell penetrating peptides having endosomal escape properties and compositions comprising such cell penetrating peptides and therapeutic agents suitable for treating ocular diseases are needed.

### SUMMARY

The present disclosure relates to, among other things, cargo conjugates and compositions containing the same, for delivering a cargo to the eye. The cargo conjugate may include a cyclic cell penetrating peptide and a therapeutic agent. The therapeutic agent may be designed to have a biological effect, for example, to treat a disease of the eye. As such, the cargo conjugates and compositions containing the same, may be used to treat ocular diseases.

The present disclosure is related to, among other things, methods of administering the cargo conjugates and compositions containing the same to a subject in need thereof. The method may include identifying a subject that may benefit from the delivery of a therapeutic agent to the eye. For example, the subject may have, or be at risk of having, an ocular disease. The method may further include delivering the cargo conjugate or composition containing the same directly to the eye. In embodiments, cargo conjugate and/or compositions containing the same are delivered to the surface of the eye. In embodiments, cargo conjugate and/or compositions containing the same are intraocularly injected. In embodiments, cargo conjugate and/or compositions containing the same are injected into the retina. In embodiments, cargo conjugate and/or compositions containing the same are delivered intravitreally.

In embodiments, a method of delivering a therapeutic agent to the eye of a subject comprises administering a therapeutically effective amount of a cargo conjugate to the eye of the subject. The cargo conjugate comprises (i) a cargo comprising the therapeutic agent that selectively binds to a target molecule associated with a disease of the eye; and (ii) an ocular delivery construct comprising an exocyclic peptide (EP), a first cyclic cell penetrating peptide (cCPP) and one or more linkers. The ocular delivery construct has the structure:

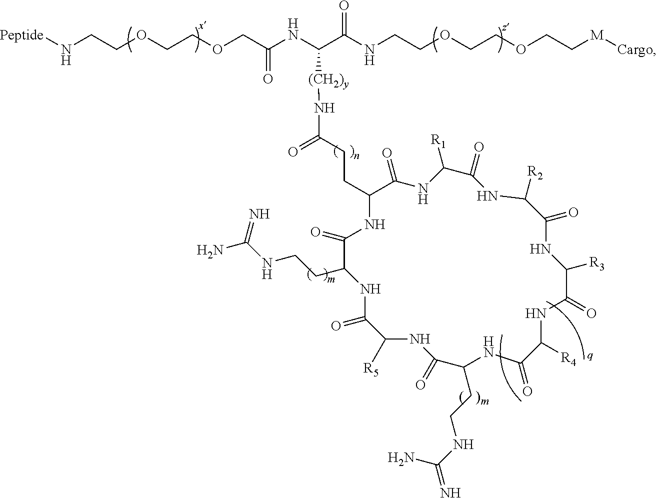

or a protonated form thereof, wherein:
R₁, R₂, and R₃ are each independently H or an aryl or heteroaryl side chain of an amino acid;
at least two of R₁, R₂, and R₃ are an aryl or heteroaryl side chain of an amino acid;
R₄ and R₆ are independently H or an amino acid side chain;
peptide is an exocyclic peptide (EP) comprising from 2 to 10 amino acids;
M is a bonding group;
each m is independently an integer from 0-3;
n is an integer from 0-2;
x′ is an integer from 0-20;
y is an integer from 1-5;
q is 1-4;
z′ is an integer from 1-23; and
Cargo is a therapeutic moiety.

In embodiments, the amino acid residue comprising an aryl or heteroaryl group is phenylalanine or 3-(2-naphtbyl)-alanine. In embodiments, R₄ and R₆ are, independently, H or a side chain of an amino acid selected from arginine, citrulline, serine or histidine. In embodiments, at least one of R₄ and R₆ are H. In embodiments, at least one of R₄ and R₆ are an amino acid side chain of arginine. In embodiments, at least one of R₄ and R₅ are an amino acid side chain of serine. In embodiments, at least one of R₄ and R₆ are an amino acid side chain of histidine.

In embodiments, the cCPP of the ocular delivery construct has a sequence selected from: FfΦRrRrQ, FGFGRGRQ; GfFGrGrQ, FfΦGRGRQ; FfFGRGRQ; FfΦGrGrQ; FGFGRRRQ; and FGFRRRRQ.

In embodiments, a method of delivering a therapeutic agent to an eye of a subject is provided, the method comprising administering a therapeutically effective amount of a cargo conjugate to the eye of the subject. The cargo conjugate comprises (i) a cargo comprising the therapeutic agent that selectively binds to a target molecule associated with a disease of the eye; and (ii) an ocular delivery construct comprising an exocyclic peptide (EP), a first cyclic cell penetrating peptide (cCPP) and one or more linkers, wherein the ocular delivery construct has the structure:

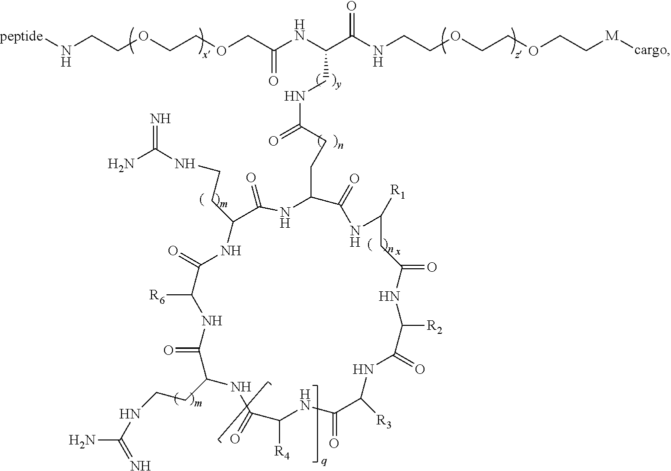

or a protonated form thereof,
wherein:
R₁, R₂, and R₃ can each independently be H or an amino acid residue having a side chain comprising an aryl or heteroaryl group;
at least two of R₁, R₂, and R₃ is an aryl or heteroaryl side chain of an amino acid;
R₄, and R₆ are independently H or an amino acid side chain;
AA_{SC} is an amino acid side chain;
nₓ is 1;
q is 1, 2, 3 or 4;
peptide is an exocyclic peptide (EP) comprising from 2 to 10 amino acids;
M is a bonding group;
each m is independently an integer from 0-3;
n is an integer from 0-2;
x′ is an integer from 0-20;
y is an integer from 1-5;
z′ is an integer from 1-23; and
Cargo is a therapeutic moiety.

In embodiments, the amino acid residue comprising an aryl or heteroaryl group is phenylalanine, beta homophenylalanine, or 3-(2-naphthyl)-alanine. In embodiments, R₄ and R₆ are, independently, H or a side chain of an amino acid selected from arginine, citrulline, serine or histidine. In embodiments, at least one of R₄ and R₆ are H. In embodiments, at least one of R₄ and Re are an amino acid side chain of arginine. In embodiments, at least one of R₄ and R₆ are an amino acid side chain of serine. In embodiments, at least one of R₄ and R₆ are an amino acid side chain of histidine.

In embodiments, the cCPP of the ocular delivery construct has a sequence selected from: βhF-FΦSRSRQ and βhF-FΦGRGRQ

In embodiments, M comprises

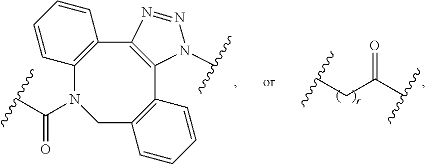

wherein t′ is 0 to 10.
In embodiments, M comprises

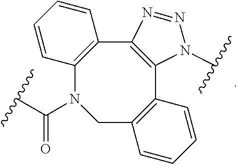 [SMILES: *C(=O)N1Cc2ccccc2-c2c(nnn2*)-c2ccccc21]

In embodiments, M comprises

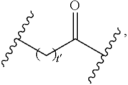

wherein t′ is 0 to 10. In embodiments, M comprises

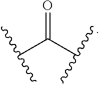 [SMILES: *C(*)=O]

In embodiments, the EP comprises 1 or 2 amino acids comprising a side chain comprising a guanidine group, or a protonated form thereof. In embodiments, the EP comprises 1, 2, 3, or 4 lysine residues. In embodiments, the EP comprises one of the following sequences: PKKKRKV; KR; RR; KKK; KGK; KBK; KBR; KRK; KRR; RKK; RRR; KKKK; KKRK; KRKK; KRRK; RKKR; RRRR; KGKK; KKGK; KKKKK; KKKRK; KBKBK; KKKRKV; PGKKRKV; PKGKRKV; PKKGRKV; PKKKGKV; PKKKRGV; or PKKKRKG. In embodiments, EP comprises PKGKRKV.

In embodiments, the cargo comprises a therapeutic oligonucleotide. In embodiments, the therapeutic oligonucleotide is an antisense oligonucleotide. In embodiments, the antisense oligonucleotide is a phosphorodiamidate morpholino oligomer (PMO). In embodiments, the cargo comprises a therapeutic peptide.

In embodiments, the disease of the eye is selected from one or more of diabetic retinopathy; glaucoma; retinitis pigmentosa; Usher syndrome; retinal tears or holes; retinal detachment, retinal ischemia; damage associated with laser therapy including photodynamic therapy; surgical light induced iatrogenic retinopathy; drug-induced retinopathies; autosomal dominant optic atrophy; toxic and/or nutritional amblyopias; Leber's hereditary optic neuropathy; atypical retinitis pigmentosa; Bardet-Biedl syndrome; blue-cone monochromacy; cataracts; central areolar choroidal dystrophy; choroideremia; cone dystrophy; rod dystrophy; rod-cone dystrophy; congenital stationary night blindness; cytomegalovirus retinitis, diabetic macular edema; dominant drusen; giant cell arteritis; Goldmann Favre dystrophy; graves' ophthalmopathy; gyrate atrophy; iritis; juvenile retinoschisis; Kearns-Sayre syndrome; Lawrence-Moon syndrome; Leber Congenital Amaurosa, wet macular degeneration; dry macular degeneration; macular dystrophy; ocularhistoplasmosis syndrome; Oguchi disease; oxidative damage; proliferative vitreoretinopathy; refsum disease; retinitis Punctata albescens; retinopathy of prematurity; rod monochromatism; Usher syndrome such as Usher Syndrome type 2A; scleritis; Sjogren-Larsson syndrome; Sorsby fundus dystrophy; Stargardt disease; choroideremia; optic neuropathy; Bietti crystalline dystrophy; Alport syndrome; X-linked retinoschisis; Macula dystrophy; Achromatopsia; congenital stationary night blindness; Best disease; Pattern dystrophy; and Doyne's honeycomb dystrophy.

In embodiments, administering a cargo conjugate to directly to an eye of the subject comprises local administration. In embodiments, local administration is selected from topical administration, intravitreal administration, periocular administration, subretinal administration, suprachoroidal administration, and intracameral administration.

In embodiments, the method further comprises identifying a subject having an ocular disease. In embodiments, the subject is suffering from an ocular disease or disorder, or is at risk of developing, an ocular disease or disorder. In embodiments, the subject has a genetic disease or disorder of the eye. In embodiments, the subject is a mammal. In embodiments, the mammal is a human.

In embodiments, administration of the compound modulates expression or activity of a target molecule. In embodiments, administration of the compound downregulates expression or activity of the target molecule. In embodiments, administration of the compound upregulates expression or activity of the target molecule. In embodiments, the target molecule is a protein. In embodiments, the target molecule is an oligonucleotide.

In embodiments, the cargo conjugate is administered in a pharmaceutical composition.

### BRIEF DESCRIPTION OF THE DRAWINGS

FIG. 1A is a schematic drawing of the eye with a section of the retina shown as schematically enlarged. Adapted from Simple Anatomy of the Retina by Helga Kolb, Webvision, the Organization of the Retina and Visual System (webvision.med.utah.edu/book/part-i-foundations/simple-anatomy-of-the-retina/).

FIG. 1B is a schematic drawing of layers and cell components of the retina. Adapted from Koeppen & Stanton: Berne and Levy Physiology, 6^{th} Edition, 2008 by Mosby, an imprint of Elsevier, Inc.

FIG. 2 shows examples of modified nucleotides used in antisense oligonucleotides described herein. Structures 1-3 (1=Phosphorothioate; 2=(SC5-Rp)-α,β-CAN; 3=PMO) are phosphate backbone modifications; 4 (2-thio-dT) is a base modification; 5-8 (5=2′-OMe-RNA, 6=2′O-MOE-RNA; 7=2′F-RNA; 8=2′F-ANA) are 2′ sugar modifications; 9-11 are constrained nucleotides; 12-14 (9=LNA; 10=(S)-cET; 11=tcDNA; 12=FHNA; 13=(S)5′-C-methyl; 14=UNA) are additional sugar modification; and 15-18 (15=E-VP; 16=Methyl phosphonate; 17=5′ phosphorothioate; 18=(S)-5′-C-methyl with phosphate) are 5′ phosphate stabilization modifications; 19=a morpholino sugar. 20 is a representation of a phosphorodiamidate morpholino oligomer backbone. Reformatted from Khvorova, A., et al., Nat. Biotechnol. 2017 March; 35(3): 238-248.

FIGS. 3A-3D illustrate examples of conjugation chemistries for connecting an antisense compound (AC), such as an antisense oligonucleotide (ASO), to a cyclic cell penetrating peptide. FIG. 3A shows amide bond formation between peptides with a carboxylic acid group or with TFP activated ester and primary amine residues at the 5′ end of an AC. FIG. 3B shows conjugation of secondary amine or primary amine modified AC at 3′ and peptide-TFP ester through amide bond formation. FIG. 3C shows conjugation of a peptide-azide to the 5′ cyclooctyne modified AC via copper-free azide-alkyne cycloaddition. FIG. 3D demonstrates another example of conjugation between a 3′ modified cyclooctyne ACs or 3′ modified azide ACs and CPP containing linker-azide or linker-alkyne/cyclooctyne moiety, via a copper-free azide-alkyne cycloaddition or cupper catalyzed azide-alkyne cycloaddition, respectively (click reaction).

FIG. 4 shows an example of conjugation chemistry for connecting an ASO and cCPP with an additional linker modality containing a polyethylene glycol (PEG) moiety.

FIG. 5 shows images of sectioned mouse eye tissue treated with a vehicle or a PMO-EEV conjugate. Tissue was stained for nuclear material (DAPI, blue) and for PMOs (anti-PMO antibody, green).

FIG. 6 shows images of sectioned mouse retinal tissue treated with a vehicle or a PMO-EEV conjugate. The tissue was stained for nuclear material (DAPI, blue) and for PMOs (anti-PMO antibody, green). The top images show both the DAPI and anti-PMO stains and the bottom images show only the anti-PMO stains. RPE=retinal pigment epithelium; ONL=outer nuclear layer; INL=inner nuclear layer; GCL=ganglion cell layer

FIG. 7 shows images of sectioned mouse retinal tissue treated with a vehicle or a PMO-EEV conjugate. The tissue was stained for nuclear material (DAPI, blue) and for glial fibrillary acidic protein (GFAP; pink).

FIG. 8 is a plot showing the percent exon skipping after mice were treated with various cargo conjugates.

### DETAILED DESCRIPTION

Methods and compositions are provided herein that can be used to transport a therapeutic moiety or therapeutic agent to the cells of the eye of a subject. Methods and compositions are provided herein may be used to treat ocular diseases.

Disclosed herein are cargo conjugates comprising an ocular delivery construct conjugated to a cargo. The cargo may be a therapeutic moiety for which delivery to a cell of the eye is desired. The therapeutic moiety may be a molecule useful for treating a disease of the eye. In embodiments, the therapeutic moiety selectively binds to a target molecule associated with an ocular disease. In embodiments, the target molecule is a macromolecule implicated in a disease or pathology of the eye. In embodiments, the target molecule is a polypeptide or protein. In embodiments, the target molecule is an oligonucleotide. In embodiments, the oligonucleotide target comprises DNA. In embodiments, the oligonucleotide target comprises genomic DNA. In embodiments, the oligonucleotide target comprises RNA. In embodiments, the oligonucleotide target comprises mRNA. In embodiments, the target is associated with an ocular disease. In embodiments, selective binding of the therapeutic moiety with the target molecule is useful for the treatment of a disease, pathology or other abnormal state or condition of the eye. In embodiments, selective binding of the therapeutic moiety to the target molecule upregulates expression or activity of the target molecule. In embodiments, selective binding of the therapeutic moiety to the target molecule downregulates expression or activity of the target molecule.

As used herein, the term “ocular delivery construct” refers to a compound that, when conjugated to cargo and ophthalmically administered, enhances uptake of the cargo in a cell of the eye relative to uptake of the cargo alone (when not conjugated to the ocular delivery construct). The ocular delivery construct may preferentially targets a tissue of the eye. In embodiments, the ocular delivery construct targets one or more structures, tissues, layers, and/or cells of eye. In embodiments, the ocular delivery construct comprises a cyclic cell penetrating peptide (cCPP); a compound comprising a cCPP and a linker; a compound comprising a cCPP and an exocyclic peptide; or a compound comprising an endosomal escape vehicle (EEV) which comprises a cCPP, an exocyclic peptide, and a linker.

In embodiments, an ocular delivery construct provided herein can be used to transport a cargo, such as a therapeutic agent, to a cell of the eye of subject. In embodiments, the ocular delivery construct transports a therapeutic moiety to an intracellular compartment of a cell. In embodiments, an ocular delivery construct transports a therapeutic moiety to the retina of a subject. In embodiments, the ocular delivery construct transports a therapeutic moiety intracellularly to cells of one or more cell types within the retina. In embodiments, the ocular delivery construct preferentially transports a therapeutic agent intracellularly to cells of one or more cell type within the retina.

In embodiments, a cargo conjugate or a composition comprising a cargo conjugate may be ophthalmically administered. As used herein, the term “ophthalmically administered,” “ophthalmic administration,” “ocularly administered,” and “ocular administration” refer to local delivery of a compound or composition directly to one or more components of the eye (described in detail later herein). Such terms exclude systematic administration of the cargo conjugate.

For ocular diseases, ocular administration of a therapeutic may allow for a more effective treatment than systematic administration. Systemic administration, in some cases, may not achieve effective concentrations of therapeutics in the eye due in part to systemic clearance mechanisms and blood-ocular barriers. Ocular administration may avoid, mitigate, or circumvent one or more systemic clearance mechanisms, such as first-pass metabolism, and/or blood-ocular barriers, such as the blood-aqueous barrier and the blood-retinal barrier to achieve therapeutically effective concentrations in cells of the eye. The blood-ocular barriers may hinder a therapeutic from penetrating the eye and/or reaching portions of the eye at a therapeutically relevant concentration when systemically administered. Additionally, ocular administration may result in fewer side effects than systemic administration of a therapeutic because ocular administration provides local delivery of the therapeutic to the tissue to be treated.

In embodiments, a cargo conjugate may be targeted to and/or localize to one or more structures, layers, and/or cell types of the eye. In embodiments, an ocularly administered cargo conjugate may be targeted to and/or localize to one or more structures, layers, and/or cell types of the eye. As used herein in the context of biodistribution of a cargo conjugate or delivery construct, the term “targeted to x” (where x is a system, such as the neuromuscular system, an organ, a structure, a layer, a tissue type, or a cell type) refers to the cargo conjugate or delivery construct preferentially interacting with x. It is understood that a cargo conjugate or delivery construct target to a particular system, organ, structure, layer, tissue type, or cell type may interact with other one or more additional systems, on or more additional organs, one or more additional tissue types, one or more additional cell types, or any combination thereof. Reference to a cargo conjugate or delivery construct targeted to x includes all the components comprising x. For example, a cargo conjugate targeted to the eye includes the cargo conjugate being targeted to one or more structures, tissues, layers, and/or cells that make up the eye.

As used herein in the context of biodistribution of a delivery construct or a cargo conjugate, the terms “localize” and “localized” refer to the physical position of the cargo conjugate within a body, an organ, a tissue, or a cell. Reference to a cargo conjugate or delivery construct being localized within a particular locale includes all the sublocales comprising the locale. For example, a cargo conjugate or delivery construct localized to the eye includes the cargo conjugate or delivery construct being localized to one or more structures, tissues, layers, and/or cells that make up the eye and can be referred to herein as an “ocular delivery construct.” An ocular delivery construct localized to the retina may be further localized to one or more of the layers and/or cells that comprise the retina.

### Anatomy of the Eye

The anatomy, physiology, and chemical composition of the eye provide barriers to delivery of therapeutic agents. Many therapeutics struggle to penetrate target tissues in the eye, such as the cornea or retina, which can result in poor bioavailability. The eye has static and dynamic barriers that inhibit therapeutic agents from reaching target tissues at therapeutically effective concentrations. Static barriers of the eye include tissues that provide a passive physical barrier to therapeutic agent penetration. Dynamic barriers include physiological clearance mechanisms and barriers that include aqueous turnover, vitreous flow, ocular drug metabolism, and eye metabolizing enzymes.

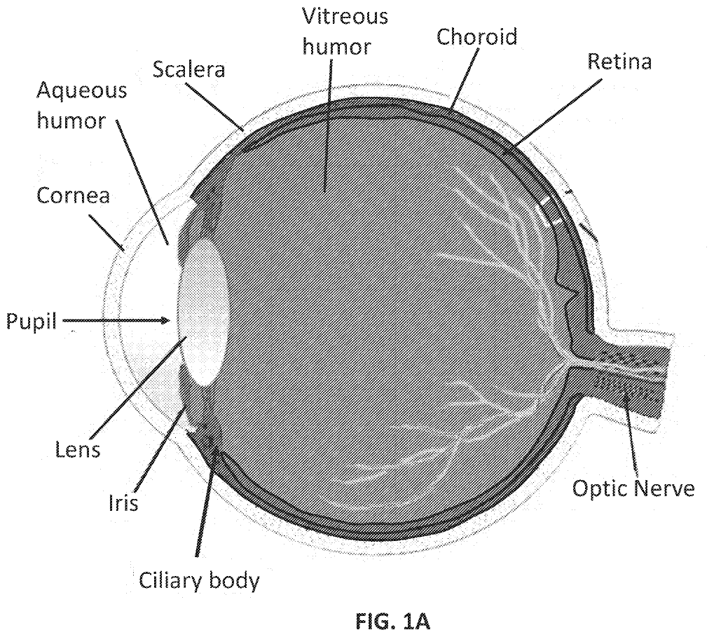

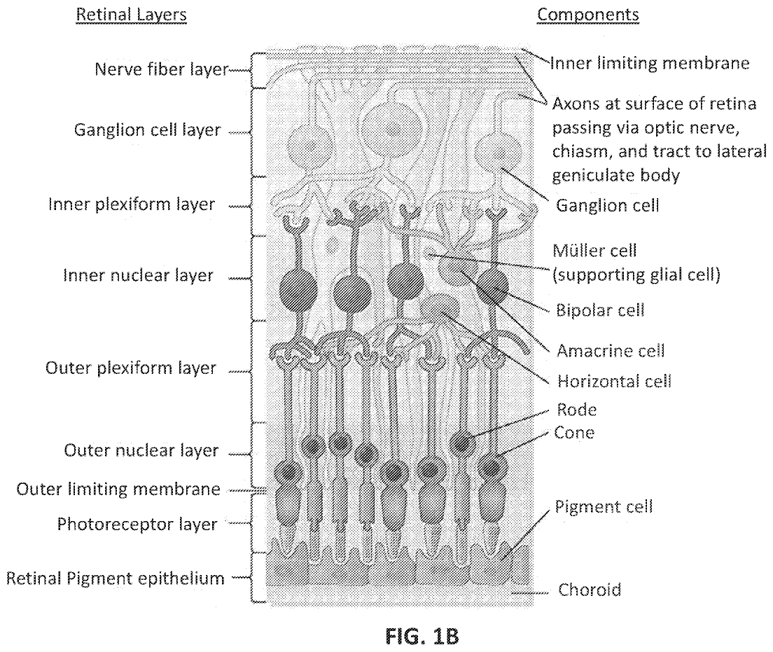

As shown in FIG. 1A, main structures in a mammalian eye include: the cornea, iris, pupil, aqueous humor, lens, vitreous humor, retina, and optic nerve. The retina is a layered structure containing distinct layers of neurons interconnected by synapses, some of which are shown in FIG. 1B. Retina layers from the anterior to posterior surface include: inner limiting membrane, nerve fiber layer (NFL), ganglion cell layer (GCL), inner plexiform layer, inner nuclear layer (INL), middle limiting membrane, outer plexiform layer, outer nuclear layer (ONL), outer (external) limiting membrane, photoreceptor layer, and the retinal pigment epithelium (RPE). The cells in the retina can be subdivided into several cell types shown in FIG. 1B: ganglion cells, Müller cells (Müller glia), bipolar cells, amacrine cells, horizontal cells, photoreceptor cells (rods and cones), and pigment cells. The majority of cell types in the retina span one, two, or three retinal layers (see FIG. 1B). Some cell types are elongated such that a first part of the cells of a cell type is in a first retinal layer, a second part of the cells of the cell type is in a second retinal layer, and a third part of the cells in the cell type is in a third retinal layer. For example, the nuclei of the photoreceptor cells are generally in the outer nuclear layer, the rods/cones of the photoreceptor cells are generally in the photoreceptor layer, and the synapses of the photoreceptor cells are generally in the outer plexiform layer.

In embodiments, an ocular delivery construct can be used to transport a therapeutic moiety (as a conjugate construct) to a structure of the eye. In embodiments, an ocular delivery construct may be used to transport a therapeutic moiety to the retina. In embodiments, the ocular delivery construct may be used to transport a therapeutic moiety to one or more retinal layer. In embodiments, the ocular delivery construct may be used to transport a therapeutic moiety to one or more of the inner limiting membrane, nerve fiber layer (NFL), ganglion cell layer (GCL), inner plexiform layer, inner nuclear layer (INL), middle limiting membrane, outer plexiform layer, outer nuclear layer (ONL), outer (external) limiting membrane, photoreceptor layer, and the retinal pigment epithelium (RPE). In embodiments, the ocular delivery construct may be used to transport a therapeutic moiety to and within one or more of a ganglion cell, a Müller cell (Müller glia), a bipolar cell, an amacrine cell, a horizontal cell, a photoreceptor cell (rods and cones), and a pigment cell.

In embodiments, when ophthalmically administered, a cargo conjugate may be targeted to and/or localize in the retina. In embodiments, the cargo conjugate may be targeted to and/or localize in one or more of the layers of the retina such as the inner limiting membrane, nerve fiber layer (NFL), ganglion cell layer, inner plexiform layer, inner nuclear layer, middle limiting membrane, outer plexiform layer, outer nuclear layer, outer (external) limiting membrane, photoreceptor layer, and retinal pigment epithelium, and/or one or more of the cell types of the retina such as a ganglion cell, a Müller cell, a bipolar cell, an amacrine cell, a horizontal cell, a photoreceptor cell (rods and cones), and a pigment cell.

### Ocular Diseases and Mechanisms of Therapeutic Moieties

The ocular delivery constructs provided herein may be conjugated to a therapeutic moiety for which delivery to the eye is desired. For example, the cargo conjugate may include a therapeutic moiety designed to provide a therapeutic effect to a subject inflicted with an ocular disease. In embodiments, the cargo conjugate includes a therapeutic moiety useful for treating a disease of the eye (i.e., an ocular disease). In embodiments, the therapeutic moiety targets a protein or gene associated with a disease of the eye. As such, the cargo conjugates and compositions may be used to treat an ocular disease. In embodiments, the therapeutic moiety modulates expression of a protein or gene associated with a disease of the eye. In embodiments, the therapeutic moiety upregulates expression of a protein or gene associated with a disease of the eye. In embodiments, the therapeutic moiety downregulates expression of a protein or gene associated with a disease of the eye. In embodiments, the therapeutic moiety modulates the composition of mRNA. In embodiments, the therapeutic moiety modulates processing of the mRNA. In embodiments, the therapeutic moiety modulates splicing of a pre-mRNA target transcript. In embodiments, the therapeutic moiety modulates alternative splicing of a pre-mRNA target transcript. In embodiments, modulation of splicing or alternative splicing induces exon skipping and/or exon inclusion.

Non-limiting examples of ocular diseases that a cargo conjugate may be used to treat include, diabetic retinopathy; glaucoma; retinitis pigmentosa (RP), such as autosomal dominant RP, autosomal recessive RP, sector RP, and RP associated with Usher syndrome; retinal tears or holes; retinal detachment; retinal ischemia; damage associated with laser therapy including photodynamic therapy; surgical light induced iatrogenic retinopathy; drug-induced retinopathies; autosomal dominant optic atrophy; toxic and/or nutritional amblyopias; Leber's hereditary optic neuropathy; atypical retinitis pigmentosa; Bardet-Biedl syndrome; blue-cone monochromacy; cataracts; central areolar choroidal dystrophy; choroideremia; cone dystrophy; rod dystrophy; rod-cone dystrophy; congenital stationary night blindness; cytomegalovirus retinitis; diabetic macular edema; dominant drusen; giant cell arteritis; Goldmann Favre dystrophy; graves' ophthalmopathy; gyrate atrophy; iritis, juvenile retinoschisis; Kearns-Sayre syndrome; Lawrence-Moon syndrome; Leber Congenital Amaurosa; wet macular degeneration; dry macular degeneration; macular dystrophy; ocularhistoplasmosis syndrome; Oguchi disease; oxidative damage; proliferative vitreoretinopathy; refsum disease; retinitis Punctata albescens; retinopathy of prematurity; rod monochromatism; Usher syndrome such as Usher Syndrome type 2A; scleritis; Sjogren-Larsson syndrome; Sorsby fundus dystrophy; Stargardt disease; choroideremia; optic neuropathy; Bietti crystalline dystrophy; Alport syndrome; X-linked retinoschisis; Macula dystrophy; Achromatopsia; congenital stationary night blindness; Best disease; Pattern dystrophy; and Doyne's honeycomb dystrophy.

In embodiments, a cargo conjugate having a therapeutic moiety designed to treat an ocular disease is provided. In embodiments, a composition comprising such a cargo conjugate is ocularly delivered to a subject in need thereof. In embodiments, the composition is administered directly the retina. In embodiments, the composition is administered intravitreally.

### Mechanisms of Action of Therapeutic Moieties

The cargo conjugate may include a therapeutic moiety (TM) for which delivery to the eye is desired, for example, to treat an ocular disease. TMs can include antisense oligonucleotides (ASO), polypeptides, and small molecules.

In embodiments, a TM is an effector that modulates target gene expression and/or target protein activity. A target gene is the gene of which modulation of gene expression and/or protein activity is desired. A target transcript is the pre-mRNA or mRNA transcript that is transcribed from the target gene. A target protein is the polypeptide or protein encoded by the target transcript. The target gene, target transcript, or target protein may be associated with a disease state. The target gene, target transcript, or target protein may be associated with an ocular disease. For example, a target gene may be a mutated gene associated with the disease state; a target transcript may have a coding sequence associated with the disease state, may be aberrantly spliced in the disease state, may be present in reduced or increased levels in the disease state, or the like; a target protein may be present in reduced or increased levels in the disease state, may have aberrant function, reduced function, or no function in the disease state, or the like; or any combination thereof. As used herein, an “ocular target” or “target molecule” can be a target gene, a target transcript, or a target protein associated with an ocular disease. In embodiments, the target gene, target transcript, and target protein are a gene, transcript, and protein associated with a disease of the retina.

A TM may exert a therapeutic effect through any suitable mechanism. In embodiments, a cargo conjugate includes a TM that modulates one or more aspects of target gene expression, such as transcription and translation, and/or target protein activity. For example, a TM may function, relative to the disease phenotype, to downregulate target gene expression; upregulate target gene expression; inhibit target protein function; increase target protein function; or any combination thereof.

In embodiments, the cargo conjugate includes a TM that modulates one or more aspects of pre-mRNA processing of a target transcript. Pre-mRNA molecules are made in the nucleus and are processed before or during transport to the cytoplasm for translation. Processing of the pre-mRNAs includes addition of a 5′ methylated cap and an approximately 200-250 base poly(A) tail to the 3′ end of the transcript. mRNA processing also includes splicing of the pre-mRNA, which occurs in the maturation of 90-95% of mammalian mRNAs. Introns (or intervening sequences) are regions of a primary transcript (or the DNA encoding it) that are not included in the coding sequence of the mature mRNA. Exons are regions of a primary transcript that remain in the mature mRNA. The exons are spliced together to form the mature mRNA sequence. Splice junctions are also referred to as splice sites with the 5′ side of the junction often called the “5′ splice site,” or “splice donor site” and the 3′ side called the “3′ splice site” or “splice acceptor site.” In splicing, the 3′ end of an upstream exon is joined to the 5′ end of the downstream exon. Thus, the unspliced RNA (or pre-mRNA) has an exon/intron junction at the 5′ end of an intron and an intron/exon junction at the 3′ end of an intron. After the intron is removed, the exons are contiguous at what is sometimes referred to as the exon/exon junction or boundary in the mature mRNA. Cryptic splice sites are those which are less often used but may be used when the usual splice site is blocked or unavailable. Cryptic splice sites may result from one or more mutations in an intron. Such mutations may result in a high occurrence of splicing at the cryptic splice site. Many eye diseases are characterized by intronic mutations that result in a high occurrence of cryptic splice site use. Alternative splicing, defined as the splicing together of different combinations of exons, often results in multiple mRNA transcripts from a single gene. A TM may modulate alternative splicing of a pre-mRNA transcript by, for example, inducing exon skipping and/or inducing exon inclusion. Induction of exon skipping and/or exon inclusion may result in a mRNA that has a different combination of exons than is observed in a mRNA transcript of a diseased state.

Some genetic mutations alter splicing which can result in the inclusion of exons not normally present in the processed mRNA and/or exclusion of exons normally present in the processed mRNA. Such inclusions and exclusions may result in a truncated protein, a protein with decreased function, a protein with no function, an unstable protein, or any combination thereof. Similarly, mutations in exons that do not necessarily affect splicing, may result in a truncated protein, a protein with decreased function, a protein with no function, an unstable protein, or any combination thereof.

In embodiments, the TM is an antisense oligonucleotide (ASO). An ASO is an oligonucleotide that binds (e.g., hybridizes) to a portion of an RNA transcript. The ASO may comprise natural or modified nucleotides. The ASO may comprise one or more modified nucleosides, one or more modified internucleoside linkages, one or more conjugate groups, or combinations thereof. In embodiments, the ASO may be used to modulate pre-mRNA processing by modulating splicing of the pre-mRNA target transcript. As used herein, “modulation of splicing,” “splicing modulation,” “modulating splicing,” and “modulate splicing” refer to altering the processing of a pre-mRNA target transcript such that the spliced mRNA transcript contains either a different combination of exons as a result of exon skipping or exon inclusion, a deletion in one or more exons, or the deletion or addition of a sequence not normally found in the spliced mRNA of the disease state (e.g., an intron sequence). Modulation of splicing may result in alternative splicing. As such modulation of splicing may result in modulation of alternative splicing. In embodiments, ASO hybridization to a target sequence in a pre-mRNA target transcript modifies splicing to produce a mature mRNA encoding a fully functional protein, a partially functional protein, or a non-functional protein. In embodiments, ASO hybridization to a target sequence in a pre-mRNA target transcript modifies splicing of a pre-mRNA in a diseased cell to produce a mature mRNA encoding a protein that is present in a non-diseased cell (e.g., a wild-type protein). In embodiments, ASO hybridization results in alternative splicing of the target pre-mRNA.

In embodiments, ASO hybridization results in skipping of one or more exons. The term “exon skipping” refers to modulation of splicing in a cell to produce a mature mRNA lacking one or more exons relative to a mature mRNA in the cell for which exon skipping has not occurred. In embodiments, an ASO induces exon skipping to produce a mature mRNA that lacks one or more exons associated with a disease state. In embodiments, an ASO induces exon skipping to produce a mature mRNA that lacks one or more pseudo-exons associated with a disease state. In embodiments, an ASO induces exon skipping to produce a mature mRNA that lacks one or more exons associated with the disease state but, as a result of the exon skipping includes one or more exons that is not present in a mature mRNA of the disease state. The one or more exons that are included may be exons present in wild-type mature mRNA that are missing in a mature mRNA in the disease state. In embodiments, the skipped exon comprises a frameshift mutation, a nonsense mutation, or a missense mutation. In embodiments, the skipped exon sequence comprises a nucleic acid deletion, substitution, or insertion. In embodiments, the skipped exon itself does not comprise a sequence mutation, but a neighboring intron or exon comprises a mutation.

In embodiments, ASO hybridization to a target sequence within a target pre-mRNA prevents inclusion of an exon sequence in the mature mRNA molecule. In embodiments, antisense oligonucleotides hybridization to a target sequence within a target pre-mRNA results in preferential expression of a wild type target protein isomer. In embodiments, antisense oligonucleotides hybridization to a target sequence within a target pre-mRNA results in expression of a re-spliced target protein comprising an active fragment of a wild type target protein.

Depending on the disease and the desired phenotype, ASOs can be designed to promote exon skipping in a target transcript to result in, for example, a protein of the healthy phenotype (full length and fully functional), a full-length protein having at least some function, a non-functional full-length protein, a fully functional truncated protein, a truncated protein having at least some function, or a non-functional truncated protein. Exon skipping may also be used to introduce a premature stop codon in a target transcript. mRNA comprising a premature stop codon may undergo nonsense mediated decay. Inducing nonsense mediate decay of a target transcript may be beneficial to treat diseases characterized by expression of a deleterious target protein or high concentrations of a target protein and/or target transcript.

An ASO can induce exon skipping by binding to and sterically inhibiting splicing of a target transcript (see, e.g., International Patent Application No. International Patent Application No. PCT/US22/28357, filed on 9 May 2022, and entitled COMPOSITIONS AND METHODS FOR MODULATING mRNA SPLICING, which application is hereby incorporated herein by reference in its entirety).

In embodiments, ASO hybridization results in exon inclusion. The term “exon inclusion” refers to modulation of splicing in a cell to produce a mature mRNA that includes one or more exons relative to a mature mRNA in the cell for which exon inclusion has not occurred. In embodiments, an ASO induces exon inclusion to produce a mature mRNA that includes one or more exons that are deleted in a disease state. In embodiments, an ASO induces exon inclusion to produce a mature mRNA that includes one or more exons whose deletion is associated with the disease state, but as a result of the exon inclusion, the mature mRNA includes the exons not present in the mature mRNA of the disease state. The one or more exons that are included may be exons present in wild-type mature mRNA that are missing in a mature mRNA in the disease state.

In embodiments, ASO hybridization to a target sequence within a pre-mRNA target transcript induces inclusion of one or more exon sequences in the mature mRNA molecule. In embodiments, ASO hybridization to a target sequence within a pre-mRNA target transcript results in preferential expression of a wild type target protein isomer. In embodiments, antisense oligonucleotides hybridization to a target sequence within a target pre-mRNA results in correction of alternative splicing observed in a disease state.

Depending on the disease and the desired phenotype, ASOs can be designed to promote intron inclusion in a target transcript to result in, for example, a protein of the healthy phenotype (full length and fully functional), a full-length protein having at least some function, a non-functional full-length protein, a fully functional truncated protein, a truncated protein having at least some function, or a non-functional truncated protein.

In embodiments, the TM may be an ASO that modulates pre-mRNA processing by inhibiting the polyadenylation of a target transcript. Inhibition of addition of a poly(A) tail to target transcript may decrease the levels of the target transcript and/or target protein levels. ASOs conjugated to cyclic peptides for modulating polyadenylation of mRNA is disclosed in International Patent Application No. PCT/US22/28354, filed on 9 May 2022, and entitled COMPOSITIONS AND METHODS FOR MODULATING GENE EXPRESSION, which application is hereby incorporated herein by reference in its entirety.

In embodiments, the TM may modulate one or more aspects of mRNA translation. In embodiments, the TM may be an ASO that prevents degradation of a mRNA target transcript. Since a single mRNA transcript is used as a translation template to produce multiple protein copies, stabilizing a target mRNA transcript may increase the number of times the transcript is used as a template, thereby increasing the levels of the protein encoded by the target transcript. Stabilization of a target mRNA transcript may increase the resistance of the target transcript to degradation (e.g., 5″-3′ and/or 3′-5′ exonuclease degradation), thereby increasing the half-life of the target transcript. Therapeutic moieties may stabilize an mRNA target transcript by binding to, for example, the 3′ untranslated region, the 5′ untranslated region, or both. Increasing the stability of an mRNA target transcript may be beneficial to treat diseases characterized by low concentrations of a protein.

In embodiments, the TM may modulate target gene expression by decreasing the stability of a mRNA target transcript and/or preventing translation of the mRNA target transcript. For example, a TM may be an ASO that sterically prevents translation of an mRNA target transcript and/or increases the susceptibility of the mRNA target transcript to decay. In embodiments, the TM may induce nonsense mediated decay of the target transcript. In embodiments the ASO comprises DNA, and binding of the ASO to the target transcript creates a DNA/RNA hybrid that can be degraded by the enzyme RNaseH. Decreasing the stability and/or preventing translation of an mRNA target transcript may be beneficial to treat diseases where reducing the amount of a target protein is desired.

In embodiments, the ASO modulates one or more aspects of target gene transcription or translation through steric blocking. The following review article describes the mechanisms of steric blocking and applications thereof and is incorporated by reference herein in its entirety: Roberts et al. Nature Reviews Drug Discovery (2020) 19: 673-694. The ASO may modulate one or more aspects of gene expression and/or protein function through various mechanisms some of which are disclosed herein.

In embodiments, the TM may modulate the activity of a target protein. In embodiments, the TM may be a polypeptide or a small molecule that functions to inhibit or increase the function of a target protein.

### Usher Syndrome

In embodiments, a cargo conjugate having a TM designed to treat Usher syndrome such as Usher syndrome type II or Usher syndrome type Ila is provided. In embodiments, a composition comprising such a cargo conjugate is ocularly delivered to a subject in need thereof, for example to treat Usher syndrome. In embodiments, the composition is administered directly the retina. In embodiments, the composition is administered intravitreally.

Usher syndrome type IIA (OMIM 276901) is the most common type of Usher syndrome and is characterized by progressive vision loss and congenital moderate hearing loss. Usherin (USH2A; NCBI Gene ID: 7399), a protein implicated in Usher syndrome type IIA, regulates the long-term maintenance of retinal photoreceptors. Pathogenic mutations in the gene that encodes usherin that disrupt usherin production, for example, by introducing a premature stop codon that results in a truncated protein and/or non-functional protein, can lead to degeneration of photoreceptors. There are currently no approved disease-modifying therapies for Usher syndrome type IIA.

Mutations in exon 13 of the USH2A gene are the most recurring mutations in Usher syndrome type IIA and include, for example, c.2299delG and c.2276G>T (See Dulla, et al., Molecular Therapy, 4 Aug. 2021, 29(8):2441-2455). Skipping of exon 13 in USH2A pre-mRNA represents a potential therapeutic strategy for treating Usher syndrome type IIA. Dulla, et al. illustrate that a functional truncated usherin results from exon skipping in the USH2A transcripts of zebrafish using antisense oligonucleotides. Furthermore, expression of human usherin (in which exon 13 of USH2A transcripts was deleted) in mouse cells in which USH2A was knocked out, resulted in a truncated version of usherin that partially restored function (see, Pendse, et al., “Exon 13-skipped USH2A protein retains functional integrity in mice, suggesting an exo-skipping therapeutic approach to treat USH2A-associated disease,” bioRxiv, 4 Feb. 2020, doi: doi.org/10.1101/2020.02.04.934240).

The following proteins and genes, which are associated with Usher syndrome, may be targeted to treat Usher syndrome: myosin VIIA (MY07A), harmonin (USH1C), cadherin 23 (CDH23), protocadherin 15 (PDCH15), USH1G, SANS, usherin (USH2A), VLGR1, GRP98, whirlin (DBFB3), and clarin-1 (USH3A).

In embodiments, a cargo conjugate includes a TM that modulates target gene expression and/or target protein activity related to one or more target genes and/or target proteins associated with Usher syndrome type IIA. In embodiments, the target gene is USH2A and the target protein is usherin. In embodiments, the TM is an ASO that modulates one or more aspects of transcription and/or translation of one or more target genes associated with Usher syndrome type IIA. In embodiments, the ASO is directed to a target nucleotide sequence within USH2A pre-mRNA and modulates one or more aspects of USH2A pre-mRNA splicing. In embodiments, ASO induces exon skipping of one or more exons of a USH2A target transcript. In embodiments, the cargo conjugate comprising the ASO induces exon skipping of exon 13 of a human USH2A target transcript. In embodiments, skipping of an exon in the USH2A pre-mRNA results in an in-frame deletion to produce a truncated usherin protein that has at least partial activity. In embodiments, skipping of exon 13 of the USH2A target transcript results in an in-frame deletion to produce a truncated usherin protein that has at least partial activity. One of skill in the art may design suitable ASO targeting a human USH2A transcript based on the sequence of the gene, which is available at ncbi.nlm.nih.gov/gene/7399, “USH2A usherin [Homo sapiens (human)],” Gene ID: 7399. In embodiments, the ASO is a phosphorodiamidate morpholino oligomer (PMO). In embodiments, the cargo conjugate comprising the ocular delivery construct and the ASO that results in skipping of one or more exons of a human USH2A target transcript is ocularly delivered to a subject in need thereof. In embodiments, the composition is administered directly the retina. In embodiments, the composition is administered intravitreally.

### Diabetic Retinopathy

In embodiments, a cargo conjugate has a TM designed to treat diabetic retinopathy (DR). In embodiments, a composition comprising such a cargo conjugate is ocularly delivered to a subject in need thereof, for example, to treat diabetic retinopathy. In embodiments, the composition is administered directly the retina. In embodiments, the composition is administered intravitreally.

Diabetic retinopathy (DR) is the leading cause of blindness in Americans between 20 and 74 years of age. Clinical and epidemiological studies have identified a genetic component to DR, but data from studies aimed at identifying genes or genome regions associated with DR have been inconsistent (see, e.g., Cho and Sobrin, Curr Diab Rep, 2014 Aug. 14(8):515). Some data suggests that erythropoietin (EPO), an angiogenic factor observed in the eye, may be linked with DR (see, ibid). Other studies suggest that transcription factor 7-like 2 (TCF7L2) may be linked with DR (see, ibid).

In embodiments, a cargo conjugate includes a TM that modulates target gene expression and/or target protein activity related to one or more target genes and/or target proteins associated with DR. In embodiments, a cargo conjugate may include a TM that modulates one or more aspects of gene expression and/or target protein activity of one or both of EPO and TCF7L2. In embodiments, the TM is an ASO.

### Glaucoma

In embodiments, a cargo conjugate has a TM designed to treat glaucoma. In embodiments, a composition comprising such a cargo conjugate is ocularly delivered to a subject in need thereof, for example, to treat glaucoma. In embodiments, the composition is administered directly the retina. In embodiments, the composition is administered intravitreally.

Glaucoma is the leading cause of blindness in the United States. Glaucoma is generally caused by damage to the optic nerve by increases in eye pressure. A number of proteins and genes have been associated with glaucoma including myocilin (MYOC), optineurin (OPTN); tank-binding kinase 1 (TBK1); ATP-binding cassette transporter A1 (ABCA1); actin filament associated protein 1 (AFAP1), GDP-Mannose 4,6-Dehydratase (GMDS); phosphomannomutase 2 (PMM2); transforming growth factor beta receptor 3 (TGFBR3); fibronectin type III domain containing 3B (FNDC3B); rho guanine nucleotide exchange factor 12 (ARHGEF12); growth arrest specific 7 (GAS7); forkhead Box C1 (FOXC1); ataxin 2 (ATXN2), thioredoxin reductase 2 (TXNRD2); ependymin related 1 (EPDR1); choline O-acetyltransferase (CHAT); GLIS family zinc finger 3 (GLIS3); FERM domain containing kindlin 2 (FERMT2); DPM2-FAM102; and P/Q-type or CaV2.1 voltage-gated calcium channel (CACNA1A) (see, e.g., Wiggs and Pasquale, Human Mol Genet, 2017 Aug. 1, 26(R1):R21-R27). In embodiments, a cargo conjugate includes a TM that modulates target gene expression and/or target protein activity related to one or more target genes and/or target proteins associated with glaucoma.

### Retinitis Pigmentosa

In embodiments, a cargo conjugate has a TM designed to treat retinitis pigmentosa (RP). In embodiments, a composition comprising such a cargo conjugate is ocularly delivered to a subject in need thereof, for example to treat RP. In embodiments, the composition is administered directly to the retina. In embodiments, the composition is administered intravitreally.

RP is characterized by the loss of photoreceptor cells over time, leading to progressive vision loss. A number of genes have been associated with RP, with the majority of them expressed in either the photoreceptors or the retinal pigment epithelium of the eye (See, e.g., Ferari et al., Curr Genomics, 2011 Jun. 12(4):238-249). For example, the following proteins and genes are associated with autosomal dominant RP: bestrophin-1 (BEST1); carbonic anhydrase IV (CA4); cone-rod homeobox protein (CRX); fascin homolog 2; actin-bundling protein (FSCN2); guanylate cyclase activator 1B (GUCA1B); inosine 5′-monophosphate dehydrogenase 1 (IMPDH1); kelch-like 7 (KLHL7); nuclear receptor subfamily 2, group E, member 3 (NR2E3); neural retina leucine zipper protein (NRL); PRP3 pre-mRNA processing factor 3 homolog (PRPF3); PRP8 pre-mRNA processing factor 8 homolog (PRPF8); PRP31 pre-mRNA processing factor 31 homolog (PRPF31); peripherin 2 (PRPH2, RDS); retinol dehydrogenase 12 (RDH12); rhodopsin (RHO), retinal outer segment membrane protein 1 (ROM1); retinitis pigmentosa 1 (RPq); RP-9 (RP9); sema domain, immunoglobulin domain (Ig) transmembrane domain I and short cytoplasmic domain of semiphorin 4A (SEMA4A); small nuclear ribonucleoprotein 200 kDa (SNRNP200); and topoisomerase I binding, arginine/serine-rich (TOPORS). In embodiments, a cargo conjugate includes a TM that modulates target gene expression and/or target protein activity related to one or more target genes and/or target proteins associated with RP.

Retinitis pigmentosa may be autosomal recessive. The following proteins and genes are associated with autosomal recessive RP: RP22; RP29; RP32; ATP-binding cassette; subfamily A (ABC1), member 4 (ABCA4); bestrophin-1 (BEST1); chromosome 2 open reading frame 71 (C2ORF71); ceramide kinase-like protein (CERKL); cyclic nucleotide gated channel alpha1 (CNGA1); cyclic nucleotide gated channel beta1 (CNGB1); crumbs homolog 1 (CRB1); eyes shut homolog (EYS, RP25); family with sequence similarity 161 member A (FAM161A); NAD(+)-specific isocitrate dehydrogenase 3 beta (IDH3B); interphotoreceptor matrix proteoglycan 2 (IMPG2); lecithin retinol acyltransferase (LRAT); C-mer proto-oncogene tyrosine kinase (MERTK); nuclear receptor subfamily 2; group E, member 3 (NR₂E3); neural retina leucine zipper protein (NRL), phosphodiesterase 6A, cGMP-specific, rod alpha (PDE6A); phosphodiesterase 6B, CGMP-specific, rod beta (PDE6B); phosphodiesterase 6G, cGMP-specific, rod gamma (PDE6G); progressive rod-cone degeneration (PRCD); prominin 1 (PROM1); retinol binding protein 3 (RBP3); retinal G protein-coupled receptor (RGR); rhodopsin (RHO); retinaldehyde-binding protein 1 (RLBP1); RP-1 protein (RP1); retinal pigment epithelium-specific 65 kDa protein (RPE65); S-antigen; retina and pineal gland (arrestin) (SAG); spermatogenesis associated protein 7 (SPATA7); tetratricopeptide repeat domain 8 (TTC8); tubby-like protein 1 (TULP1); usher syndrome 2a (USH2A); and zinc finger protein 513 (ZNF513). In embodiments, a cargo conjugate includes a TM that modulates target gene expression and/or target protein activity related to one or more target genes and/or target proteins associated with autosomal recessive RP.

Retinitis pigmentosa may be X-linked. The following proteins and genes are associated with X-linked RP: retinitis pigmentosa 2 protein (RP2); RP6; RP23; RP24; RP34; and retinitis pigmentosa GTPase regulator (RPGR). In embodiments, a cargo conjugate includes a TM that modulates target gene expression and/or target protein activity related to one or more target genes and/or target proteins associated with X-linked RP.

Retinitis pigmentosa may be autosomal dominant Autosomal dominate RP (adRP) is caused by heterozygous mutations in the PRFPF31 gene. The PRPF31 gene encodes human pre-mRNA processing factor 3 (PRPF31). PRPF31 is a ubiquitous pre-mRNA splicing factor that is a part of the small nuclear ribonucleoprotein complex of the spliceosome. More than 130 genetic PRPF31 mutations are known, some of which are loss of function variants. Penetrance of PRPF31-associated adRP is incomplete; that is, carriers may be asymptomatic but still pass the disease to offspring. The major determinant of PRPF31 mutation penetrance is thought to be the expression level of the nonmutant PRPF31 allele (See Ali-Nasser Et. al., Mol Vis, 2022; 28, pg 359-368, PMID: 36338669, PMCID: PMC9603903). Carriers that have a high expression level of the nonmutant PRPF31 protein may not be symptomatic. Wild-type PRPF31 gene expression is highly variable and impacted by several factors such as CCR4-NOT.

CCR4-NOT (transcription complex subunit 3; also called CNOT3) is a multi-protein structure that is a negative regulator of PRPF31 transcription (see, PLOS Genet 8(11): e1003040 doi: 10.1371/journal.pgen.1003040). CCR4-NOT binds to the promoter of PRPF31 inhibiting transcription of PRPF31. CCR4-NOT is encoded by the CNOT3 gene. Modulating the activity and/or level of the CCR4-NOT t protein and/or the level of CNOT3 transcripts may be used to modulate PRPF31 gene expression.

In embodiments, a cargo conjugate is provided that includes a TM that modulates target gene expression and/or target protein activity related to one or more target genes and/or target proteins associated with adRP. In embodiments, the target gene or target protein may be PRPF31 (PRPF31), CNOT3 (CCR4-NOT), or both. In embodiments, the TM increases PRPF31 target transcript and/or protein levels. In embodiments, the TM decreases CCR4-NOT target protein levels and/or CNOT3 target transcript levels which may result in the increase of PRPF31 transcript levels and/or PRPF31 protein levels. In embodiments, the TM induces nonsense mediate decay of CNOT3 target transcripts thereby reducing the level of CNOT3 target transcripts and/or CCR4-NOT target protein levels. In embodiments, the TM is an ASO that hybridizes to at least a portion of a CNOT3 target transcript to induce nonsense mediated decay of the CNOT3 target transcript.

### Bardet-Biedl Syndrome

In embodiments, a cargo conjugate has a TM designed to treat Bardet-Biedl syndrome. In embodiments, a composition comprising such a cargo conjugate is ocularly delivered to a subject in need thereof, for example to treat Bardet-Biedl syndrome. In embodiments, the composition is administered directly the retina. In embodiments, the composition is administered intravitreally.

Bardet-Biedl syndrome is characterized rode and/or cone dystrophy. A number of proteins and genes have been associated with Bardet-Biedl syndrome including BBS1, BBS2, ARL6 (BBS3), BBS4, BBS5, MKKS (BBS7), TTCB (BBS8), PTHB1 (BBS9), BBS10, TRIM32 (BBS11), BBS12, MKS1 (BBS13), CEP290/NPNP6/LCA10 (BBS14), WDPCP/FRITZ (BBS15), SDCCAG8 (BBS16), LZTFL1 (BBS17), BBIP1/10 (BBS18), IFT27 (BBS19), and AZI1/CEP131 (BBS20) (See, Priya, et al., Indian J Opthamol, 2016 September, 64(9):620-627). In embodiments, a cargo conjugate includes a TM that modulates target gene expression and/or target protein activity related to one or more target genes and/or target proteins associated with Bardet-Biedl syndrome.

### Autosomal Dominant Optic Atrophy

In embodiments, a cargo conjugate has a TM designed to treat autosomal dominant optic atrophy (ADOA). In embodiments, a composition comprising such a cargo conjugate is ocularly delivered to a subject in need thereof, for example, to treat ADOA. In embodiments, the composition is administered directly the retina. In embodiments, the composition is administered intravitreally.

Autosomal dominant optic atrophy (ADOA) is the most common hereditary optic neuropathy (heritable in an autosomal dominant manner). Symptoms of ADOA commonly present in early childhood, and vision loss progresses throughout the lifetime of the patient. In addition to progressive visual failure, ADOA has also been associated with sensorineural deafness, ataxia, myopathy, and external ophthalmoplegia. ADOA often impacts retinal ganglion cells and the axon forming the optic nerve.

ADOA is associated with mutations in the OPA1 gene. The OPA1 (3q38-q29) gene encodes the OPA1 protein, a ubiquitously expressed mitochondrial GTPase that is involved in regulating mitochondrial function such as, for example, oxidative phosphorylation, mitochondrial DNA maintenance, and apoptosis. The OPA1 gene includes 30 exons which encode OPA1 pre-cursor proteins. The OPA1 pre-cursor proteins are targeted to the mitochondria and cleaved into OPA1 long-form proteins which become anchored to the inner mitochondrial membrane, or OPA1 short-form proteins which are soluble within the cell. In humans, there are at least eight OPA1 protein variants due to differential splicing of exon 4, 4b, and 5b.

Over 400 OPA1 genetic mutations have been reported, some of which result in the expression of a less-functional or non-functional OPA1 protein. The truncated proteins often lack a complete GTPase domain. Some OPA1 genetic mutations are thought to result in OPA1 haploinsufficiency (see Kushnareva, et al., Cell Death Dis. 2016 July; 7(7): e2309, doi: 10.1038/cddis.2016.160). Increasing the global expression of OPA1 (from both alleles) may allow for an increase in the levels of full-length functional OPA1.

In embodiments, a cargo conjugate includes a TM that modulates target gene expression and/or target protein activity related to one or more target genes and/or target proteins associated with ADOA. In embodiments, the target gene is OPA1. In embodiments, the TM acts to increase OPA1 target transcript levels which may increase in functional OPA1 protein levels. In embodiments, the TM is an ASO. In embodiments, the ASO is a PMO. In embodiments, the TM may be used to target the 5′ UTR and/or the 3′ UTR of a OPA1 target transcript to stabilize the transcript. One of skill in the art may design suitable antisense oligonucleotides targeting the gene and/or the transcript of human OPA1 based on the sequence of the gene, which is available at ncbi.nlm.nih.gov/gene/4976, “OPA1 mitochondrial dynamin like GTPase [Homo sapiens (human)],” Gene ID: 4976.

### Leber Congenital Amaurosis

In embodiments, a cargo conjugate has a TM designed to treat Leber congenital amaurosis (LCA). In embodiments, a composition comprising such a cargo conjugate is ocularly delivered to a subject in need thereof, for example to treat LCA. In embodiments, the composition is administered directly the retina. In embodiments, the composition is administered intravitreally.

Leber congenital amaurosis (LCA) is a form of inherited retinal degradation causing blindness or visual impairment, often before the age of one. LCA is a heterogenous disease in which mutants of at least 15 genes are associated with LCA. CEP290 (15%), GUCY2D (12%) and CRB1 (10%) are the most frequently mutated LCA genes (See Prog Retin Eye Res. 2008 July; 27(4):391-419. Doi: 10.1016/j.preteyeres.2008.05.003). At least 35 different mutations have been identified in The CEP290 gene that are associated with LCA. The gene product of CEP290 is the centrosomal protein 290 kDA protein (CEP290; also known as nephrovystin-6 (NPHP6), MKS3, and BBS14). CEP290 plays a role in the centrosome. CEP290 also plays a role in cilia development, such as the primary cilia of the photoreceptor cells of the retina. In embodiments, a cargo conjugate includes a TM that modulates target gene expression and/or target protein activity related to one or more target genes and/or target proteins associated with LCA. In embodiments, the target gene is CEP290.

A common hypomorphic (partial loss of gene function through reduced transcription and/or translation) is located within intron 26 of a CEP290 transcript (c.2991+1655A>G) (see Collin, et al., Molecular Therapy-Nucleic Acids (2012) 1, e14; doi: 10.1038/mtna.2012.3). This mutation creates a cryptic slice donor site within intron 26. The cryptic splice site results in the inclusion of an aberrant 128 base pair exon into the transcript and introduces a pre-mature stop codon (p.C998X) into the transcript. Ultimately, patients with this mutation low levels (or none) of the full-length functional CEP290 protein.

In embodiments, the TM acts to increase full-length CEP290 transcript levels. The full-length CEP290 transcripts lack the aberrant 128 base pair exon addition and the pre-mature stop codon. In embodiments, the TM induces exon skipping of the aberrant 128 base pair exon in a CEP290 target transcript to restore normal splicing and result in an increase of full-length CEP290 transcript levels and/or protein levels. In embodiments, the TM is an ASO that induces exon skipping by targeting the cryptic splice donor site to sterically block splicing factors from binding to the CEP290 target transcript to complete the cryptic splicing. In embodiments, the ASO is a PMO. One of skill in the art may design suitable antisense oligonucleotides targeting the transcript or gene of human CEP290 based on the sequence of the gene, which is available at ncbi.nlm.nih.gov/gene/80184, “entrosomal protein 290 [Homo sapiens (human)],” Gene ID: 80184.

### Macular Dystrophy/Stargardt Disease

In embodiments, a cargo conjugate has a TM designed to treat macular dystrophy. In embodiments, a composition comprising such a cargo conjugate is ocularly delivered to a subject in need thereof, for example to treat glaucoma. In embodiments, the composition is administered directly the retina. In embodiments, the composition is administered intravitreally.

Macular dystrophy is a group of diseases that cause deterioration of the retina. Examples of macular dystrophy diseases include Stargardt disease, Vitelliform macular dystrophy, and North Carolina macular dystrophy. Stargardt disease (STGD) is the most common form of macular dystrophy. STGD is characterized by the buildup of lipofuscin (lipid containing residues of lysosomal digestion) on the macula. The accumulation of lipofuscin damages the cells responsible for sharp central vision.

There are three known subtypes of STGD; STGD1, STGD2, and STDG3. STDG1 is caused by mutations in the ABCA4 gene. The ABCA4 gene encodes an ATP-binding cassette transporter subfamily A member 4 protein (ABCA4; See ABCA4 ATP binding cassette subfamily A member 4 [Homo sapiens (human)], Gene ID NO; 24, ncbi.nlm.nih.gov/gene/24). ABCA4 is involved in the visual cycle. More specifically, ABC4A is involved in the clearance of trans-retinal and excess 11-cis retinal/11-cis-retinal Schiff-base conjugates from photoreceptor cells to prevent the buildup of toxic bisretinoid compounds (See Quazi and Molday, PNAS, vol 111, no 13, pg. 5024-5029, doi: 10.1073/pnas. 1400780111). Dysfunctional ABCA4 may result in accumulation of bisretinoid compounds which can damage cells. Common mutations in the ABC4 gene include c.5461-101T>C and c.4539_2001G>A (See, e.g., Dulla, et al., Investigative Opthalmology & Visual Science, July 2018, Vol., 59, 5315; and Garanto, Et. Al., Genes (Basel), 2019 Jun. 14; 10(6):452, doi: 10.3390/genes10060452).

In embodiments, a cargo conjugate includes a TM that modulates target gene expression and/or target protein activity related to one or more target genes and/or target proteins associated with STGD, such as STGD1, STGD2, and STDG3. In embodiments, the target gene is ABCA4. In embodiments, the TM is an ASO. In embodiments, the ASO is a PMO.

### Other Diseases

A number of other genes and proteins associated with other diseases of the eye are known and can be readily identified through, for example, a literature search. The cargo conjugates may include a TM that targets such genes or gene products.

### Therapeutic Moieties

The cargo conjugates provided herein comprises an ocular delivery construct and a therapeutic moiety or therapeutic agent. The therapeutic moiety may be any suitable therapeutic moiety for treating a disease of the eye. As used herein, the terms “therapeutic moiety” or “therapeutic agent” refer to any molecule (e.g., polypeptide, small molecule, oligonucleotide, gene editing machinery) that is designed to have and/or has prophylactic or other biological activity. In embodiments, the therapeutic moiety selectively binds to a target molecule associated with an ocular disease. In embodiments, the target molecule is a macromolecule implicated in a disease or pathology of the eye. In embodiments, the target molecule is a polypeptide or protein. In embodiments, the target molecule is an oligonucleotide. In embodiments, the oligonucleotide target comprises DNA. In embodiments, the oligonucleotide target comprises genomic DNA. In embodiments, the oligonucleotide target comprises RNA. In embodiments, the oligonucleotide target comprises mRNA. In embodiments, the target molecule is associated with an ocular disease. In embodiments, selective binding of the therapeutic moiety with the target molecule is useful for the treatment of a disease, pathology or other abnormal state or condition of the eye. In embodiments, selective binding of the therapeutic moiety to the target molecule upregulates expression or activity of the target molecule. In embodiments, selective binding of the therapeutic moiety to the target molecule downregulates expression or activity of the target molecule.

In embodiments, the therapeutic moiety comprises a therapeutic oligonucleotide. In embodiments, the therapeutic moiety comprises a polypeptide. In embodiments, the therapeutic moiety comprises a small molecule. In embodiments, the therapeutic moiety includes one or more components of gene editing machinery.

### Therapeutic Oligonucleotides

In embodiments, the therapeutic moiety comprises a therapeutic oligonucleotide. In embodiments, the therapeutic oligonucleotide comprises an antisense oligonucleotide (ASO). In embodiments, the therapeutic oligonucleotide comprises siRNA, RNAi, microRNA, antagomir, an aptamer, a ribozyme, an immunostimulatory oligonucleotide, a decoy oligonucleotide, a supermir, a miRNA mimic, a miRNA inhibitor, or a combination thereof (See, for example, Chery, J., “RNA therapeutics: RNAi and antisense mechanisms and clinical applications,” Postdoc J, July 2016, 4(7):35-50, and Zhu, et al., “RNA-based therapeutics: an overview and prospectus: Cell Death & Disease, 23 Jul. 2022, 12(644) (doi: 10.1038/s41419-022-05075-2).

In embodiments, therapeutic oligonucleotides are provided that include from about 5 to about 100 nucleic acids in length. In embodiments, the therapeutic oligonucleotide is from about 5 to about 50, about 8 to about 40, about 10 to about 30, about 15 to about 30, or about 20 to about 30 nucleotides in length. In embodiments, the therapeutic oligonucleotide includes one or more modified nucleosides, one or more modified internucleoside linkages, one or more conjugate groups, or combinations thereof.

### Antisense Oligonucleotides (ASO)

In embodiments, the therapeutic oligonucleotide is an antisense oligonucleotide (ASO) directed to a target gene or a target transcript associated with a disease of the eye. The ASO may be directed to and bind to a target nucleotide sequence located within a target gene or a target transcript. In embodiments, the target nucleotide sequence is within a target gene and/or target transcript associated with an eye disease.

The term “antisense oligonucleotide” refers to an oligonucleotide that is at least partially complementary to a target sequence within a target polynucleotide. An antisense oligonucleotide (ASO) is a single stranded molecule that contains DNA, RNA, or combinations or modifications thereof that are at least partially complementary to a chosen sequence, e.g., a target nucleotide sequence within a target gene or target transcript.

An ASO may modulate one or more aspects of gene expression and/or protein function via hybridization of the ASO with a target nucleotide sequence. The ASO may modulate one or more aspects of gene expression and/or protein function through various mechanisms. For example, hybridization of an ASO to a target nucleotide sequence of a target transcript may modulate splicing of a target transcript such as, for example, via exon skipping; exon inclusion; alternative splicing; prevent polyadenylation of the target transcript; increase the target transcript stability; induce target transcript degradation; prevent translation of the target transcript; or any combination thereof. ASOs have been demonstrated to be effective and targeted inhibitors of protein synthesis, and, consequently, can be used to modulate gene expression of a targeted gene.

In embodiments, the ASO hybridizes with a target sequence of a target gene or target transcript having sequence from about 5 to about 50 nucleotides in length, which can also be referred to as the length of the ASO. In embodiments, the ASO is from about 5 to about 50, about 8 to about 40, about 10 to about 30, about 15 to about 30, or about 20 to about 30 nucleotides in length. In embodiments, the ASO is at least about 5, about 6, about 7, about 8, about 9, about 10, about 11, about 12, about 13, about 14, or about 15, and up to about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 nucleotides in length. In embodiments, the ASO is about 15 nucleotides in length. In embodiments, the ASO is about 16 nucleotides in length. In embodiments, the ASO is about 17 nucleotides in length. In embodiments, the ASO is about 18 nucleotides in length. In embodiments, the ASO is about 19 nucleotides in length. In embodiments, the ASO is about 20 nucleotides in length. In embodiments, the ASO is about 21 nucleotides in length. In embodiments, the ASO is about 22 nucleotides in length. In embodiments, the ASO is about 23 nucleotides in length. In embodiments, the ASO is about 24 nucleotides in length. In embodiments, the ASO is about 25 nucleotides in length. In embodiments, the ASO is about 26 nucleotides in length. In embodiments, the ASO is about 27 nucleotides in length. In embodiments, the ASO is about 28 nucleotides in length. In embodiments, the ASO is about 29 nucleotides in length. In embodiments, the ASO is about 30 nucleotides in length.

In embodiments, the ASO may be less than about 100 percent complementary to a target nucleotide sequence. As used herein, the term “percent complementarity” refers to the number of nucleobases of an ASO that have nucleobase complementarity with a corresponding nucleobase of target nucleotide sequence by the total length (number of nucleobases) of the ASO. One skilled in the art recognizes that the inclusion of mismatches is possible without eliminating the activity of the antisense compound. In embodiments, the ASOs contain no more than about 15%, no more than about 10%, no more than 5%, or no mismatches. In embodiments, the ASOs are at least about 80%, at least about 85%, at least about 90%, at least about 95%, at least about 96%, at least about 97%, at least about 98%, at least about 99%, about 100%, or 100% complementary to a target nucleic acid. Percent complementarity of an oligonucleotide is calculated by dividing the number of complementary nucleobases by the total number of nucleobases of the oligonucleotide. Percent complementarity of a region of an oligonucleotide is calculated by dividing the number of complementary nucleobases in the region by the total number of nucleobases region.

In embodiments, incorporation of nucleotide affinity modifications allows for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between a an ASO and a target nucleotide sequence, such as by determining melting temperature (Tm). Tm or change in Tm (ΔTm) can be calculated by techniques that are familiar to one of ordinary skill in the art. For example, techniques described in Freier et al. (Nucleic Acids Research, 1997, 25, 22:4429-4443) allow one of ordinary skill in the art to evaluate nucleotide modifications for their ability to increase the melting temperature of an RNA:DNA duplex.

### Therapeutic Oligonucleotides Design

Design of a therapeutic oligonucleotides (e.g., an ASO) will depend upon the target gene. Targeting a therapeutic oligonucleotide to a particular target nucleotide sequence can be a multistep process. The process usually begins with the identification of gene of interest. The transcript of the gene of interest is analyzed and a target nucleotide sequence is identified. In embodiments, the target gone is a gene associated with a disease of the eye.

One of skill in the art will be able to design, synthesize, and screen therapeutic oligonucleotides of different nucleobase sequences to identify a sequence that results in antisense activity. For example, a therapeutic oligonucleotide can be designed that inhibits expression of a target gene. Methods for designing, synthesizing, and screening therapeutic nucleotides for antisense activity against a preselected target nucleic acid and/or target gene can be found, for example in “Antisense Drug Technology, Principles, Strategies, and Applications” Edited by Stanley T. Crooke, CRC Press, Boca Raton, Florida, which is incorporated by reference in its entirety for any purpose.

The efficacy of a therapeutic oligonucleotide (e.g., an ASO) may be assessed by evaluating the antisense activity effected by their administration. As used herein, the term “antisense activity” refers to any detectable and/or measurable activity attributable to the hybridization of a therapeutic oligonucleotide to its target nucleotide sequence. Such detection and/or measuring may be direct or indirect. In embodiments, antisense activity is assessed by detecting and or measuring the amount of target protein in a cell or population of cells before and after administration of the therapeutic oligonucleotide to the cell or population of cells. In embodiments, antisense activity is assessed by detecting and/or measuring the amount of target transcript in a cell or population of cells.

### Therapeutic Oligonucleotides Structure

Therapeutic oligonucleotides comprise nucleosides linked through internucleoside linkages. Nucleosides include a pentose sugar (e.g., ribose or deoxyribose) and a nitrogenous base (nucleobase or simply base) covalently attached to sugar. The naturally occurring (traditional) bases found in DNA and/or RNA are adenine (A), guanine (G), thymine (T), cytosine (C), and uracil (U). The naturally occurring (traditional) sugars found in DNA and/or RNA deoxyribose (DNA) and ribose (RNA). A naturally occurring (traditional) nucleoside linkage is a phosphodiester bond. In embodiments, the therapeutic oligonucleotides may have all natural sugars, natural bases, and natural internucleoside linkages.

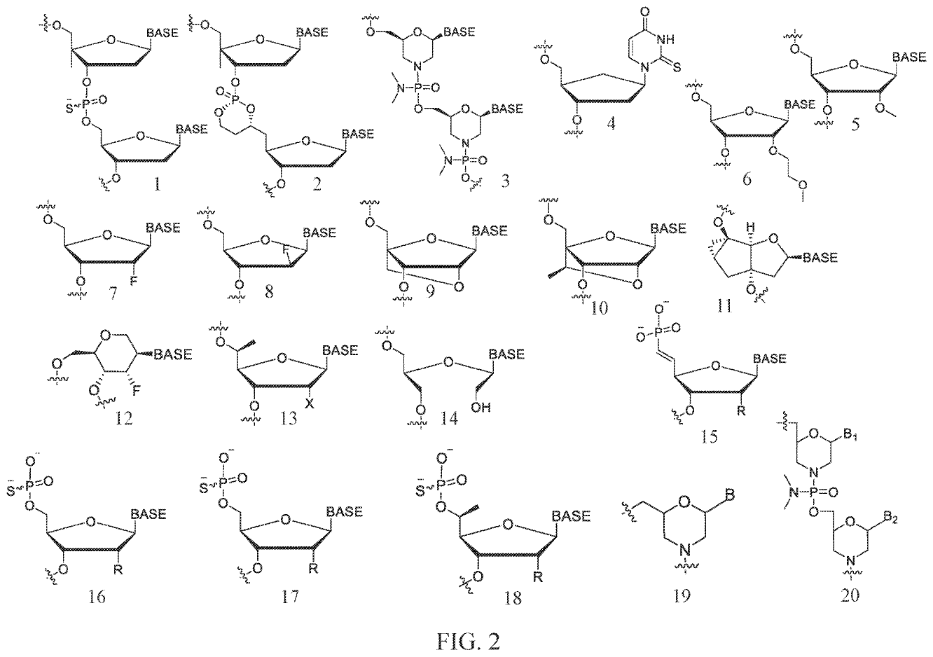

Chemically modified nucleosides are routinely used for incorporation into therapeutic oligonucleotides to enhance one or more properties, such as nuclease resistance, pharmacokinetics, or affinity for a target gene or target transcript. Non-limiting examples of nucleosides are provided in FIG. 2 and in Khvorova et al. Nature Biotechnology (2017) 35:238-248, which is incorporated by reference herein in its entirety. In embodiments, a therapeutic oligonucleotide has one or more modified nucleosides. In embodiments, a therapeutic oligonucleotide has one or more modified sugars. In embodiments, a therapeutic oligonucleotide has one or more modified bases. In embodiments, a therapeutic oligonucleotide has one or more modified internucleoside linkages.

In general, a nucleobase is any group that contains one or more atoms or groups of atoms capable of hydrogen bonding to a base of another nucleic acid. In addition to natural nucleobases, many modified nucleobases or nucleobase mimetics known to those skilled in the art are amenable with the compounds described herein. The terms modified nucleobase and nucleobase mimetic can overlap, but generally a modified nucleobase refers to a nucleobase that is fairly similar in structure to the parent nucleobase, such as for example a 7-deaza purine, a 5-methyl cytosine, or a G-clamp, whereas a nucleobase mimetic generally includes more complicated structures, such as for example a tricyclic phenoxazine nucleobase mimetic. Methods for preparation of the above noted modified nucleobases are well known to those skilled in the art.

In embodiments, a therapeutic oligonucleotide includes one or more nucleosides having a modified sugar moiety. In embodiments, the furanosyl sugar ring of a natural nucleoside can be modified. A furanosyl sugar ring may be modified in any suitable manner, including, but not limited to, addition of a substituent group, bridging of two non-geminal ring atoms to form a bicyclic nucleic acid (BNA) and substitution of an atom or group such as —S—, —N(R)— or —C(R₁)(R₂) for the ring oxygen at the 4′-position. Modified sugar moieties are well known and can be used to alter, typically increase, the affinity of the antisense compound for its target and/or increase nuclease resistance. A representative list of modified sugars includes, but is not limited to, non-bicyclic substituted sugars, especially non-bicyclic 2′-substituted sugars having a 2′-F, 2′-OCH3 or a 2′-O(CH2)2-OCH3 substituent group, and 4′-thio modified sugars. Sugars can also be replaced with a sugar mimetic group, for example, a morpholino ring, a methylenemorpholine ring, among others.

In embodiments, a therapeutic oligonucleotide may include one or more bicyclic modified sugars (BNA's), such as, for example, LNA (4′-(CH2)-O-2′ bridge), 2′-thio-LNA (4′-(CH2)-S-2′ bridge), 2′-amino-LNA (4′-(CH2)-NR-2′ bridge), ENA (4′-(CH2)2-O-2′ bridge), 4′-(CH2)3-2′ bridged BNA, 4′-(CH2CH(CH3))-2′ bridged BNA″ cEt (4′-(CH(CH3)-O-2′ bridge), and cMOE BNAs (4′-(CH(CH2OCH3)-O-2′ bridge).

In embodiments, a therapeutic oligonucleotide may include one or more locked nucleic acids” (LNAs) in which the 2′-hydroxyl group of the ribosyl sugar ring is linked to the 4′ carbon atom of the sugar ring thereby forming a 2′-C,4′-C-oxymethylene linkage to form the bicyclic sugar moiety. The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Internucleoside linking groups link the nucleosides or otherwise modified monomer units of an oligonucleotide together. The two main classes of internucleoside linking groups are defined by the presence or absence of a phosphorus atom. Representative phosphorus containing internucleoside linkages include, but are not limited to, phosphodiesters, phosphotriesters, methylphosphonates, phosphoramidate, phosphorodiamidate, and phosphorothioates. Representative non-phosphorus containing internucleoside linking groups include, but are not limited to, methylenemethylimino (—CH2-N(CH3)-O—CH2-), thiodiester (—O—C(O)—S—), thionocarbamate (—O—C(O)(NH)—S—); siloxane (—O—Si(H)2-O—); and N,N′-dimethylhydrazine (—CH2-N(CH3)-N(CH3)-). Therapeutic oligonucleotides having non-phosphorus internucleoside linking groups are referred to as oligonucleosides. Modified internucleoside linkages, compared to natural phosphodiester linkages, can be used to alter, typically increase, nuclease resistance of the therapeutic oligonucleotides. Internucleoside linkages having a chiral atom can be prepared racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

In embodiments, a phosphate group can be linked to the 2′, 3′ or 5′ (or 6′, for a 6 membered ring, such as a methylenemorpholine ring) hydroxyl moiety of the sugar (or sugar mimetic). In forming oligonucleotides, the phosphate groups covalently link adjacent nucleosides to one another to form a linear polymeric compound. Within oligonucleotides, the phosphate groups are commonly referred to as forming the internucleoside backbone of the oligonucleotide. The normal linkage or backbone of RNA and DNA is a 3′ to 5′ phosphodiester linkage. In embodiments, the oligonucleotide is a phosphorodiamidate morpholino oligomer (PMO) comprising a backbone of methylenemorpholine rings linked through phosphorodiamidate internucleoside linkages.

PMOs are uncharged nucleic acid analogs bind to target nucleic acid through base paring. PMOs that bind to mRNA may block interaction of proteins to the mRNA through steric blockade (See, e.g., Nan and Zhang, Front. Microbiol. 20 Apr. 2019 (doi.org/10.3389/fmicb.2018.00750)). As uncharged, or net neutral charged, oligonucleotides, PMOs are particularly effective for intracellular delivery with ocular delivery construct.

The therapeutic oligonucleotides may contain one or more asymmetric centers and thus give rise to enantiomers, diastereomers, and other stereoisomeric configurations that may be defined, in terms of absolute stereochemistry, as (R) or (S); α or β; or as (D) or (L). Included in the antisense compounds provided herein are all such possible isomers, as well as their racemic and optically pure forms.

In embodiments, therapeutic oligonucleotides are modified by covalent attachment of one or more conjugate groups. In general, conjugate groups modify one or more properties of the attached therapeutic oligonucleotides including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as a therapeutic oligonucleotides. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In embodiments, the conjugate group is a polyethylene glycol (PEG), and the PEG is conjugated to either the therapeutic oligonucleotide, a linker, an EP, or the cyclic peptide.

### Gene-Editing Machinery

In embodiments, the therapeutic moiety comprises one or more component of gene-editing machinery. As used herein, “gene-editing machinery” refers to protein, nucleic acids, or combinations thereof, which may be used to edit a genome. Non-limiting examples of gene-editing machinery include guide RNAs (gRNAs), nucleases, nuclease inhibitors, and combinations and complexes thereof.

The gene editing machinery may be used to repair a mutated gene or to introduce a mutation into a gene. The gene may be a gene associated with a disease of the eye.

In embodiments, a linker conjugates the ocular delivery construct to the one or more components of gene-editing machinery. Any linker described in this disclosure or that is known to a person of skill in the art may be utilized.

gRNA

In embodiments, the therapeutic moiety includes a guide RNA (gRNA). A gRNA targets a genomic loci in a prokaryotic or eukaryotic cell.

In embodiments, the gRNA is a single-molecule guide RNA (sgRNA). A sgRNA includes a spacer sequence and a scaffold sequence. A spacer sequence is a short nucleic acid sequence used to target a nuclease (e.g., a Cas9 nuclease) to a specific nucleotide region of interest (e.g., a genomic DNA sequence to be cleaved). In embodiments, the spacer may be about 17-24 bases in length, such as about 20 bases in length.

In embodiments, the spacer targets a site that immediately precedes a 5′ protospacer adjacent motif (PAM). The PAM sequence may be selected based on the desired nuclease. For example, the PAM sequence may be any one of the PAM sequences shown in Table 1 below, wherein N refers to any nucleic acid, R refers to A or G, Y refers to C or T, W refers to A or T, and V refers to A or C or G.

TABLE 1
Nucleases and PAM sequences

| PAM sequence (5′ to 3′) | Nuclease | Isolated from |
|---|---|---|
| NGG | SpCas9 | Streptococcus pyogenes |
| NGRRT or NGRRN | SaCas9 | Staphylococcus aureus |
| NNNNGATT | NmeCas9 | Neisseria meningitidis |
| NNNNRYAC | CjCas9 | Campylobacter jejuni |
| NNAGAAW | StCas9 | Streptococcus thermophiles |
| TTTV | LbCpf1 | Lachnospiraceae bacterium |
| TTTV | AsCpf1 | Acidaminococcus sp. |

In embodiments, a spacer may target a sequence of a mammalian gene, such as a human gene. In embodiments, the spacer may target a mutant gene. In embodiments, the spacer may target a coding sequence. In embodiments, the spacer may target an exonic sequence. In embodiments, the spacer may target a polyadenylation site (PS). In embodiments, the spacer may target a sequence element of a PS. In embodiments, the spacer may target a polyadenylation signal (PAS), an intervening sequence (IS), a cleavage site (CS), a downstream element (DES), or a portion or combination thereof. In embodiments, a spacer may target a splicing element (SE) or a cis-splicing regulatory element (SRE).

The scaffold sequence is the sequence within the sgRNA that is responsible for nuclease (e.g., Cas9) binding. The scaffold sequence does not include the spacer/targeting sequence. In embodiments, the scaffold may be about 10 to about 150 nucleotides in length, or about 50 to about 100 nucleotides in length.

In embodiments, the gRNA is single guide RNA molecule comprising the spacer and the scaffold. In embodiments, the gRNA comprises two molecules that hybridize to form the gRNA. An example of a gRNA that includes two molecules is a gRNA comprising a crRNA and tracrRNA. In embodiments, the gRNA or one or more components thereof may further include a poly(A) tail.

In embodiments, a compound that includes a CPP is conjugated to a nucleic acid that includes a gRNA or a component thereof. In embodiments, the nucleic acid includes about 1, about 2, about 3, about 4, about 5, about 6, about 7, about 8, about 9, about 10, about 11, about 12, about 13, about 14, about 15, about 16, about 17, about 18, about 19, or about 20 gRNAs or components thereof. In embodiments, the gRNAs recognize the same target. In embodiments, the gRNAs recognize different targets.

In embodiments, a compound that includes a CPP is conjugated to a nucleic acid designed to express the gRNA within a cell. The nucleic acid may include a promoter sequence to drive expression of the gRNA.

### Nuclease

In embodiments, the therapeutic moiety includes a nuclease. In embodiments, the nuclease is a Type II, Type V-A, Type V-B, Type VC, Type V-U, Type VI-B nuclease. In embodiments, the nuclease is a transcription, activator-like effector nuclease (TALEN), a meganuclease, or a zinc-finger nuclease or a modified form or variant thereof. In embodiments, the nuclease is a Cas9, Cas12a (Cpf1), Cas12b, Cas12c, Tnp-B like, Cas13a (C2c2), Cas13b, or Cas14 nuclease or a modified form or variant thereof. For example, in embodiments, the nuclease is a Cas9 nuclease or a Cpf1 nuclease.

In embodiments, the ocular delivery construct is conjugated to a nucleic acid encoding a nuclease. In embodiments, the nucleic acid encoding a nuclease includes a sequence encoding a promoter, wherein the promoter drives expression of the nuclease.

gRNA and Nuclease Combinations

In embodiments, the therapeutic moiety includes a ribonucleoprotein (RNP) that includes a gRNA and a nuclease. A RNP is a complex of a gRNA bound to a nuclease. The gRNA, the nuclease, or both may be covalently attached to an ocular delivery construct to form a cargo conjugate having an RNP therapeutic moiety.

In embodiments, a composition that includes: (a) a cargo conjugate comprising an ocular delivery construct conjugated to a gRNA and (b) a nuclease is delivered to a cell. In embodiments, a composition that includes: (a) a cargo conjugate comprising an ocular delivery construct conjugated to a nuclease and (b) an gRNA is delivered to a cell. In embodiments, a composition that includes: (a) a first cargo conjugate comprising a first delivery construct conjugated to a gRNA and (b) a second cargo conjugate comprising a second delivery construct conjugated to a nuclease is delivered to a cell. In embodiments, the first delivery construct and the second delivery construct are the same. In embodiments, the first delivery construct and the second delivery construct are different.

In embodiments, a cargo conjugate comprises an ocular delivery construct conjugated to a nucleic acid encoding a gRNA and/or a nuclease. In embodiments, the nucleic acid encoding a nuclease and a gRNA includes a sequence encoding a promoter, wherein the promoter drives expression of the nuclease and the gRNA. In embodiments, the nucleic acid encoding a nuclease and a gRNA includes two promoters, wherein a first promoter controls expression of the nuclease and a second promoter controls expression of the gRNA. In embodiments, the nucleic acid encoding a gRNA and a nuclease encodes from about 1 to about 20 gRNAs, or from about 1, about 2, about 3, about 4, about 5, about 6, about 7, about 8, about 9, about 10, about 11, about 12, about 13, about 14, about 15, about 16, about 17, about 18, or about 19, and up to about 20 gRNAs. In embodiments, the gRNAs recognize different targets. In embodiments, the gRNAs recognize the same target.

### Nuclease Inhibitors

In embodiments, the therapeutic moiety includes a nuclease inhibitor. A limitation of gene editing is potential off-target editing. The delivery of a nuclease inhibitor may limit off-target editing. In embodiments, the nuclease inhibitor is a polypeptide, polynucleotide, or small molecule.

### Therapeutic Polypeptides

In embodiments, the therapeutic moiety includes a therapeutic polypeptide. In embodiments, the therapeutic polypeptide includes a peptide inhibitor. In embodiments, the peptide inhibitor inhibits a protein associated with a disease of the eye. In embodiments, the therapeutic polypeptide includes a peptide replacement therapy to functionally replace an aberrantly expressed protein associated with a disease of the eye.

In embodiments, the therapeutic moiety includes a protein or a fragment thereof. In embodiments, the therapeutic moiety includes an RNA binding protein or an RNA binding fragment thereof. In embodiments, the therapeutic moiety includes an enzyme. In embodiments, the therapeutic moiety includes an RNA-cleaving enzyme or an active fragment thereof.

### Antibodies

In embodiments, the therapeutic moiety includes an antibody or an antigen-binding fragment. Antibodies and antigen-binding fragments can be derived from any suitable source, including human, mouse, camelid (e.g., camel, alpaca, llama), rat, ungulates, or non-human primates (e.g., monkey, rhesus macaque).

The term “antibody” includes intact polyclonal or monoclonal antibodies and antigen-binding fragments thereof. For example, a native immunoglobulin molecule includes two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein that includes two heterologous polypeptide chains). The two heterodimeric proteins then associate by virtue of additional interchain disulfide bonds between the heavy chain polypeptides to form an immunoglobulin protein or polypeptide.

In embodiments, the therapeutic moiety is an antigen-binding fragment that binds to a target protein associated with a disease of the eye. An antibody may modulate the activity of the target protein to which it binds. In embodiments, the therapeutic moiety is an antigen-binding fragment that binds to a target transcript of a protein (Ye et al., PNAS (2008), 105(1):82-87; and Jung et al., (RNA (2014), 20(6):805-814). In embodiments, an antigen-binding fragment that binds to a target protein includes 1, 2, 3, 4, 5, or all 6 CDRs of a variable heavy chain (VH) and/or a variable light chain (VL) sequence from an antibody that specifically binds to the target protein. In embodiments, the antigen binding fragment includes 1, 2, or 3 of the CDRs of a camelid single domain antibody such as the VHH region. In embodiments, the antigen-binding fragment that binds to a target protein is a portion of a full-length antibody, such as Fab, F(ab′)2, Fab′, Fv fragments, minibodies, diabodies, single domain antibody (dAb), single-chain variable fragments (scFv), multispecific antibodies formed from antibody fragments, or any other modified configuration of the immunoglobulin molecule that includes an antigen-binding site or fragment of the required specificity.

In embodiments, the therapeutic moiety includes a bispecific antibody. Bispecific antibodies (BsAbs) are antibodies that can simultaneously bind two separate and unique antigens (or different epitopes of the same antigen). In embodiments, the therapeutic moiety includes a bispecific antibody that can simultaneously bind to a target protein associated with an eye disease and another target protein. Non-limiting examples include scFv (single-chain variable fragment), BsDb (bispecific diabody), scBsDb (single-chain bispecific diabody), scBsTaFv (single-chain bispecific tandem variable domain), DNL-(Fab)3 (dock-and-lock trivalent Fab), sdAb (single-domain antibody), and BssdAb (bispecific single-domain antibody).

BsAbs with an Fc region are useful for carrying out Fc mediated effector functions such as antibody-dependent cell-mediated cytotoxicity and complement-dependent cytotoxicity. They have the half-life of normal IgG. On the other hand, BsAbs without the Fc region (bispecific fragments) rely solely on their antigen-binding capacity for carrying out therapeutic activity. Due to their smaller size, these fragments have better solid-tumor penetration rates. BsAb fragments do not require glycosylation, and they may be produced in bacterial cells. The size, valency, flexibility and half-life of BsAbs to suit the application.

In embodiments, the therapeutic moiety includes a “diabody.” The term diabody refers to a bispecific antigen-binding antibody fragment in which VH and VL domains are expressed in a single polypeptide chain using a linker that is too short to allow for pairing between the two domains on the same chain, thereby forcing the domains to pair with complementary domains of another chain and creating two antigen-binding sites (see, e.g., Holliger et al., Proc. Natl. Acad. Sci. USA 90:6444-48 (1993) and Poljak et al., Structure 2:1121-23 (1994)). Diabodies may be designed to bind to two distinct antigens and are bi-specific antigen binding constructs.

In embodiments, the therapeutic moiety includes a “nanobody” or a “single domain antibody” (which can also be referred to herein as sdAbs or VHH). Single domain antibody refers to an antigen-binding fragment that includes a single monomeric variable antibody domain comprising one variable domain (VH) of a heavy-chain antibody. In embodiments, the variable chain is the VHH of a camelid single chain antibody.

In embodiments, the therapeutic moiety includes a minibody.

In embodiments, the therapeutic moiety is an antibody mimetic. Antibody mimetics are compounds that, like antibodies, can specifically bind antigens, but that are not structurally related to antibodies. They are usually artificial peptides or proteins with a molar mass of about 3 to 20 kD (compared to the molar mass of antibodies at ~150 kDa). Examples of antibody mimetics include affibody molecules affilins, affimers, affitins, alphabodies anticalins, avimers, DARPins, fynomers Kunitz domain peptides and monobodies.

### Other Peptides

In embodiments, the therapeutic moiety includes a peptide. In embodiments, the peptide acts as an agonist, increasing the activity of a target protein. In embodiments, the peptide acts as an antagonist, decreasing the activity of a target protein. In embodiments, the peptide is configured to inhibit protein-protein interaction (PPI). Protein-protein interactions (PPIs) are important in many biochemical processes, including transcription of nucleic acid and various post-translational modifications of translated proteins. PPIs can be experimentally determined by biophysical techniques such as X-ray crystallography, NMR spectroscopy, surface plasma resonance (SPR), bio-layer interferometry (BLI), isothermal titration calorimetry (ITC), radio-ligand binding, spectrophotometric assays and fluorescence spectroscopy. Peptides that inhibit protein-protein interaction can be referred to as peptide inhibitors.

In embodiments, the therapeutic moiety includes a peptide inhibitor. In embodiments, the peptide inhibitor includes from about 5 to about 100 amino acids, from about 5 to about 50 amino acids; from about 15 to about 30 amino acids; or from about 20 to about 40 amino acids. In embodiments, the peptide inhibitor includes one or more chemical modifications, for example, to reduce proteolytic degradation and/or to improve in vivo half-life. In embodiments, the peptide inhibitor includes one or more synthetic amino acids and/or a backbone modification. In embodiments, the peptide inhibitor has an α-helical structure.

In embodiments, the peptide inhibitor is configured to disrupt one or more function of a protein associated with a disease of the eye. In embodiments, the peptide inhibitor is configured to disrupt formation of protein complexes. In embodiments, binding of the peptide inhibitor to the protein blocks dimer formation.

### Small Molecules

In embodiments, the therapeutic moiety includes a small molecule for treating a disease of the eye. In embodiments, the small molecule does not readily gain access to an intracellular compartment of a cell of the eye when delivered by itself (not conjugated to an ocular delivery construct).

### Ocular Delivery Constructs

A cargo conjugate includes an ocular delivery construct. An ocular delivery construct may be used to transport a cargo (e.g., a therapeutic moiety) to a structure of the eye, an eye tissue, and/or a cell-type of the eye. In embodiments, an ocular delivery construct may be used to transport a cargo across a cell membrane, for example, to deliver the cargo to the cytosol or nucleus of a cell in the eye. An ocular delivery construct can comprise a cyclic cell penetrating peptide (cCPP); a cCPP and a linker; a cCPP and an exocyclic peptide (EP); or an endosomal escape vehicle (EEV) which comprises a cCPP, an EP, and a linker.

The configuration of the ocular delivery construct depends at least in part on the components of the ocular delivery construct. Two or more components that are coupled, conjugated, or linked are a part of the same compound. In embodiments, the ocular delivery construct comprises a cCPP, and the cCPP is conjugated to the cargo to from a cargo conjugate. In embodiments, the ocular delivery construct comprises a cCPP and a linker, and the cCCP is coupled to the linker and the linker is conjugated to the cargo to form a cargo conjugate. In embodiments, the ocular delivery construct comprises a cCPP and an EP, and the cCPP is coupled to the EP and the EP is conjugated to the cargo to from the cargo conjugate; the cCPP is coupled to the EP and the cCPP is conjugated to the cargo to form the cargo conjugate; or both the cCPP and the EP are conjugated to the cargo to form the cargo conjugate. In embodiments, the ocular delivery construct comprises an EEV, and the EP is coupled to the cCPP, the cCPP is coupled to the linker, and the linker is conjugated to the cargo; the cCPP is coupled to the EP, the EP is coupled to the linker, and the linker is conjugated to the cargo; the cCPP is coupled to the linker, the EP is coupled to the linker, and the EP is conjugated to the cargo, the EP is coupled to the linker, the cCPP is coupled to the linker, and the cCPP is conjugated to the cargo; or the EP is coupled to the linker, the cCPP is coupled to the linker, and the linker is conjugated to the cargo to from a cargo conjugate.

### Cell Penetrating Peptides (CPP)

The ocular delivery constructs comprise at least one cell penetrating peptide (CPP). The cell penetrating peptide can be a cyclic cell penetrating peptide (cCPP). In embodiments, the ocular delivery constructs comprise one cell penetrating peptide (cCPP). In embodiments, the ocular delivery constructs comprise two cell penetrating peptides (cCPP). In embodiments, the cCPP is capable of penetrating a cell membrane. In embodiments, the cCPP can deliver the cargo to the cytosol of the cell. The cCPP can deliver the cargo to a cellular location where a target gene, target transcript, and/or target protein is located. To conjugate the cCPP to a cargo, an EP, and/or a linker, at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP can comprise 6 to 10 amino acids. By way of example, cCPP comprising 6-10 amino acid residues can have a structure according to any of Formula I-A to I-E:

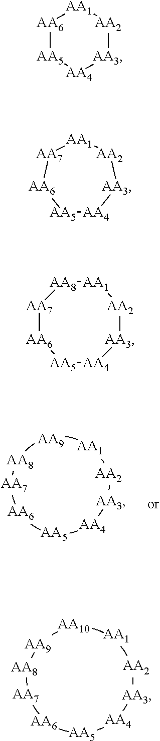

wherein AA₁, AA₂, AA₃, AA₅, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. Abbreviations used herein for some natural and non-natural amino acids are shown in Table 2.

As used herein, the term “amino acid” refers to compounds having an amino group and a carboxylic acid group. Most amino acids (except for glycine) also have a side chain. As used herein, “amino acid side chain” or “side chain” refers to the characterizing substituent bound to the α-carbon of the amino acid.

An “α-amino acid” is an amino acid in which the amino group is attached to the first (alpha) carbon adjacent to the carboxylic acid group, such that the carbon atom of the carbonyl is separated from the nitrogen atom of the amino group by one carbon atom. A “b-amino acid” (also called “beta-amino acid,” and “β-amino acid”) is an analog of an α-amino acid in which the amino group is attached to the second (beta) carbon, rather than the alpha-carbon, such that the carbon atom of the carbonyl is separated from the nitrogen atom of the amino group by two carbon atoms. Examples of b-amino acids include but are not limited to b-alanine and b-homophenylalanine.

An “uncharged” amino acid is an amino acid that does not have a charge at a physiological pH (between 5.0 and 8.0). It is noted that histidine can exist in neutral or positively charged forms at physiological pH.

A side chain that does not comprise an aryl or heteroaryl group, can be referred to herein as a “non-aryl” side chain. In embodiments, the side chain that does not comprise an aryl or heteroaryl group can be uncharged and is referred to herein as an uncharged, non-aryl side chain. Amino acids with uncharged non-aryl amino side chains include, but are not limited to, histidine, threonine; serine; leucine; isoleucine, valine; neopentylglycine, alanine; homoalanine; homoserine; 3-(4-thiazolyl)-alanine; 3-(4-furanyl)-alanine; 3-(4-thienyl)-alanine; and b-amino acid derivatives thereof.

The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, alloisoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof.

TABLE 2
Amino Acid Abbreviations

| | Abbreviations* | Abbreviations* |
|---|---|---|
| Amino Acid | L-amino acid | D-amino acid |
| Alanine | Ala (A) | ala (a) |
| Allo-isoleucine | Aile | Aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| Glutamic acid | Glu (E) | glu (e) |
| Glutamine | Gln (Q) | gln (q) |
| Glycine | Gly (G) | gly (g) |
| Histidine | His (H) | his (h) |
| Homoproline (aka pipecolic acid) | Pip (Θ) | pip (θ) |
| Isoleucine | Ile (I) | ile (i) |
| Leucine | Leu (L) | leu (l) |
| Lysine | Lys (K) | lys (k) |
| Methionine | Met (M) | met (m) |
| 3-(2-naphthyl)-alanine; 2-naphthylalanine | Nal; (Φ); (L-Nal or 2- | Nal; (φ); dNal, |
| | Nal) | dnal; D-Nal; or |
| | | 2-nal |
| 3-(1-naphthyl)-alanine; 1-naphthylalanine | 1-Nal | 1-nal |
| Norleucine | Nle (Ω) | nle |
| Phenylalanine | Phe (F) | phe (f) |
| Phenylglycine | Phg (Ψ) | phg |
| 4-(phosphonodifluoromethyl)phenylalanine | F₂Pmp (Λ) | f₂pmp |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |
| Beta-homophenylalanine | βhF, βh-F, B-hF, b-hF, | βhf, βh-f, Bhf, |
| | bhF | Bh-f, bhf, b-hf, |
| | | bh-f |

*single letter abbreviations: capital letters indicate the L-amino acid form, lower case letter indicate the D-amino acid form.

In embodiments, the cCPP can comprise 2 contiguous amino acids with hydrophobic side chains. In embodiments, the cCPP can comprise 3 contiguous amino acids with hydrophobic side chains. The hydrophobicity of amino acid residues can be measured and/or calculated using a variety of techniques. In embodiments, the hydrophobicity of an amino acid residue can be determined by calculating its consensus value on the consensus scale of D. Eisenberg et al., using the method described in D. Eisenberg et al., “Hydrophobic Moments and Protein Structure,” Faraday Symp. Chem. Soc. 1982, 17, 109-120 (e.g., D. Eisenberg et al.). A hydrophobic amino acid is an amino acid that has a hydrophobic side chain.

In embodiments, one or two amino acids in the cCPP can have no side chain. In embodiments, all amino acids in the cCPP have a side chain. As used herein, when no side chain is present, the amino acid has two hydrogen atoms on the carbon atom(s) (e.g., —CH₂) linking the amine and carboxylic acid of the amino acid residue. The amino acid having no side chain can be glycine or beta-alanine.

The cCPP can comprise from 6 to 20, from 6 to 10, or from 6 to 8 amino acid residues, wherein: (i) at least two amino acids can, independently, be glycine, b-alanine, serine, histidine or 4-aminobutyric acid; (ii) at least two amino acids can have a side chain comprising an aryl or heteroaryl group; and (iii) at least two amino acid has a side chain comprising a guanidine group, or a protonated form thereof. In embodiments, (i) two amino acids can, independently, be glycine, b-alanine, serine, histidine or 4-aminobutyric acid; (ii) two or three amino acids can have a side chain comprising an aryl or heteroaryl group; and (iii) two amino acid has a side chain comprising a guanidine group, or a protonated form thereof.

In embodiments, one amino acid of the cCPP can be glycine, b-alanine, serine, histidine, or 4-aminobutyric acid. In embodiments, two amino acids can be, independently, glycine, b-alanine, serine, histidine, or 4-aminobutyric acid. In embodiments, three amino acids can be glycine, b-alanine, serine, histidine, or 4-aminobutyric acid.

In embodiments, one amino acid of the cCPP can have a side chain comprising an aryl or heteroaryl group. In embodiments, two amino acids of the cCPP can have a side chain comprising an aryl or heteroaryl group. In embodiments, three amino acids of the cCPP can have a side chain comprising an aryl or heteroaryl group.

In embodiments, one amino acid of the cCPP can have a side chain that does not comprise an aryl or heteroaryl group, referred to herein as a “non-aryl” side chain. In embodiments, the side chain that does not comprise an aryl or heteroaryl group can be uncharged and is referred to herein as an uncharged, non-aryl side chain. In embodiments, two amino acids of the CPP (e.g., cCPP) can have an uncharged, non-aryl side chain. In embodiments, three amino acids of the CPP (e.g., cCPP) can have an uncharged, non-aryl side chain. Amino acids with uncharged non-aryl amino side chains include, but are not limited to, histidine; threonine; serine; leucine; isoleucine; valine; neopentylglycine; alanine; homoalanine; homoserine; 3-(4-thiazolyl)-alanine, 3-(4-furanyl)-alanine; and 3-(4-thicnyl)-alanine.

In embodiments, one amino acid of the cCPP has a side chain comprising a guanidine group, or a protonated form thereof. In embodiments, two amino acids of the cCPP can have a side chain comprising a guanidine group, or a protonated form thereof. In embodiments, three amino acids of the cCPP can have a side chain comprising a guanidine group, or a protonated form thereof. In embodiments, four amino acids of the cCPP can have a side chain comprising a guanidine group, or a protonated form thereof.

In embodiments, the cCPP can comprise 4 to 20 amino acids, such as 6 to 20 amino acids, wherein: (i) at least one amino acid has a side chain comprising a guanidine group, or a protonated form thereof; (ii) at least one amino acid has no side chain or a side chain comprising

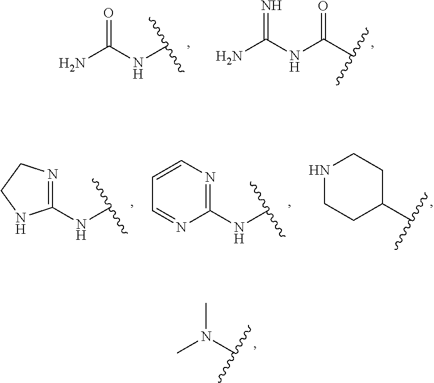

or a protonated form thereof; and (iii) at least two amino acids independently have a side chain comprising an aryl or heteroaryl group.

In embodiments, at least two amino acids can have no side chain or a side chain comprising

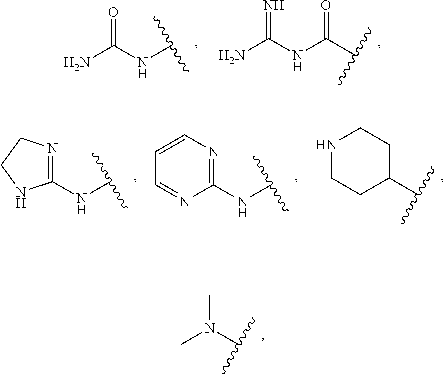

or a protonated form thereof. As used herein, when no side chain is present, the amino acid has two hydrogen atoms on the carbon atom(s) (e.g., —CH₂—) linking the amine and carboxylic acid.

In embodiments, the amino acid having no side chain can be glycine or β-alanine.

In embodiments, the cCPP can comprise from 6 to 20 amino acid residues, wherein: (i) at least two amino acids can be glycine, β-alanine, or 4-aminobutyric acid residues; (ii) at least two amino acids can have a side chain comprising an aryl or heteroaryl group; and (iii) at least two amino acids can have a side chain comprising a guanidine group,

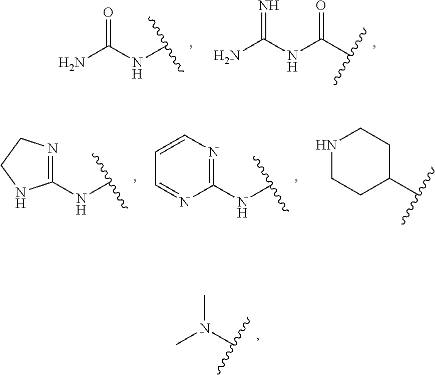

or a protonated form thereof.

In embodiments, the cCPP can comprise from 6 to 20 amino acid residues, wherein: (i) 2 amino acids can independently be glycine, β-alanine, or 4-aminobutyric acid residues; (ii) 2 or 3 amino acids can have a side chain comprising an aryl or heteroaryl group; and (iii) 2 amino acids have a side chain comprising a guanidine group,

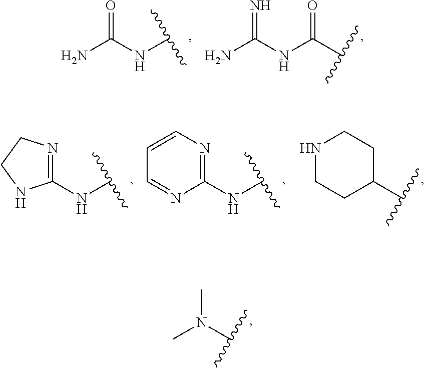

or a protonated form thereof.

In embodiments, the cCPP can comprise from 6 to 20 amino acid residues, wherein: (i) at least three amino acids can independently be glycine, β-alanine, or 4-aminobutyric acid residues; (ii) at least one amino acid can have a side chain comprising an aryl or heteroaryl group; and (iii) at least one amino acid can have a side chain comprising a guanidine group,

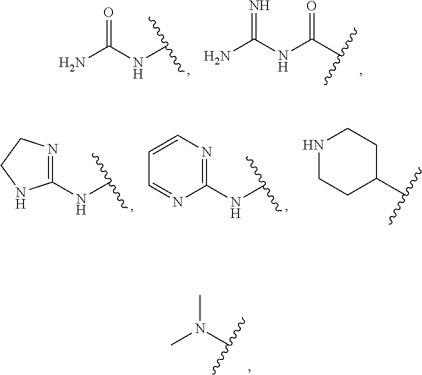

or a protonated form thereof.

### Glycine and Related Amino Acid Residues

In embodiments, the cCPP can comprise (i) 1, 2, 3, 4, 5, or 6 glycine, β-alanine, 4-aminobutyric acid residues, or combinations thereof. In embodiments, the cCPP can comprise (i) 2 glycine, β-alanine, 4-aminobutyric acid residues, or combinations thereof. In embodiments, the cCPP can comprise (i) 3 glycine, β-alanine, 4-aminobutyric acid residues, or combinations thereof. In embodiments, the cCPP can comprise (i) 4 glycine, β-alanine, 4-aminobutyric acid residues, or combinations thereof. In embodiments, the cCPP can comprise (i) 5 glycine, β-alanine, 4-aminobutyric acid residues, or combinations thereof. In embodiments, the cCPP can comprise (i) 6 glycine, β-alanine, 4-aminobutyric acid residues, or combinations thereof. In embodiments, the cCPP can comprise (i) 3, 4, or 5 glycine, β-alanine, 4-aminobutyric acid residues, or combinations thereof. In embodiments, the cCPP can comprise (i) 3 or 4 glycine, β-alanine, 4-aminobutyric acid residues, or combinations thereof.

In embodiments, the cCPP can comprise (i) 1, 2, 3, 4, 5, or 6 glycine residues. The cCPP can comprise (i) 2 glycine residues. In embodiments, the cCPP can comprise (i) 3 glycine residues. In embodiments, the cCPP can comprise (i) 4 glycine residues. In embodiments, the cCPP can comprise (i) 5 glycine residues. In embodiments, the cCPP can comprise (i) 6 glycine residues. In embodiments, the cCPP can comprise (i) 3, 4, or 5 glycine residues. In embodiments, the cCPP can comprise (i) 3 or 4 glycine residues. In embodiments, the cCPP can comprise (i) 2 or 3 glycine residues. In embodiments, the cCPP can comprise (i) 1 or 2 glycine residues.

In embodiments, the cCPP can comprise (i) 3, 4, 5, or 6 glycine, β-alanine, 4-aminobutyric acid residues, or combinations thereof. In embodiments, the cCPP can comprise (i) 3 glycine, β-alanine, 4-aminobutyric acid residues, or combinations thereof. In embodiments, the cCPP can comprise (i) 4 glycine, β-alanine, 4-aminobutyric acid residues, or combinations thereof. In embodiments, the cCPP can comprise (i) 5 glycine, β-alanine, 4-aminobutyric acid residues, or combinations thereof. In embodiments, the cCPP can comprise (i) 6 glycine, β-alanine, 4-aminobutyric acid residues, or combinations thereof. In embodiments, the cCPP can comprise (i) 3, 4, or 5 glycine, β-alanine, 4-aminobutyric acid residues, or combinations thereof. In embodiments, the cCPP can comprise (i) 3 or 4 glycine, β-alanine, 4-aminobutyric acid residues, or combinations thereof.

In embodiments, the cCPP can comprise at least three glycine residues. In embodiments, the cCPP can comprise (i) 3, 4, 5, or 6 glycine residues. In embodiments, the cCPP can comprise (i) 3 glycine residues. In embodiments, the cCPP can comprise (1) 4 glycine residues. In embodiments, the cCPP can comprise (i) 5 glycine residues. In embodiments, the cCPP can comprise (i) 6 glycine residues. In embodiments, the cCPP can comprise (i) 3, 4, or 5 glycine residues. The cCPP can comprise (i) 3 or 4 glycine residues.

In embodiments, none of the glycine, β-alanine, or 4-aminobutyric acid residues in the cCPP are contiguous. In embodiments, two or three glycine, β-alanine, 4- or aminobutyric acid residues can be contiguous. In embodiments, two glycine, β-alanine, or 4-aminobutyric acid residues can be contiguous.

In embodiments, none of the glycine residues in the cCPP are contiguous. For example, each glycine residues in the cCPP can be separated by an amino acid residue that is not glycine.

In embodiments, two or more of the glycine residues in the cCPP are contiguous. In embodiments, two or three glycine residues are contiguous. In embodiments, two glycine residues are contiguous

Amino Acid Side Chains with an Aryl or Heteroaryl Group

In embodiments, the cCPP can comprise (ii) 2, 3, 4, 5 or 6 amino acid residues independently having a side chain comprising an aryl or heteroaryl group. In embodiments, the cCPP can comprise (ii) 2 amino acid residues independently having a side chain comprising an aryl or heteroaryl group. In embodiments, the cCPP can comprise (ii) 3 amino acid residues independently having a side chain comprising an aryl or heteroaryl group. In embodiments, the cCPP can comprise (ii) 2, 3, or 4 amino acid residues independently having a side chain comprising an aryl or heteroaryl group. In embodiments, the cCPP can comprise (ii) 2 or 3 amino acid residues independently having a side chain comprising an aryl or heteroaryl group.

In embodiments, the cCPP can comprise (ii) 2, or 3 amino acid residues independently having a side chain comprising an aryl group. In embodiments, the cCPP can comprise (ii) 2 amino acid residues independently having a side chain comprising an aryl group. In embodiments, the cCPP can comprise (ii) 3 amino acid residues independently having a side chain comprising an aryl group. In embodiments, the cCPP can comprise (ii) 2 or 3 amino acid residues independently having a side chain comprising an aryl group.

The aryl group can be a 6- to 14-membered aryl. Aryl can be phenyl, naphthyl or anthracenyl, each of which is optionally substituted. Aryl can be phenyl or naphthyl, each of which is optionally substituted. The heteroaryl group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aryl or heteroaryl group can each independently be bis(homonaphthylalanine); homonaphthylalanine; naphthylalanine; phenylglycine; bis(homophenylalanine); homophenylalanine, phenylalanine, tryptophan, or tyrosine, each of which is optionally substituted with one or more substituents. The amino acid having a side chain comprising an aryl or heteroaryl group can each independently be selected from: naphthylalanine; homophenylalanine and phenylalanine.

The amino acid residue having a side chain comprising an aryl or heteroaryl group can each be independently a residue of phenylalanine, naphthylalanine, phenylglycine, homophenylalanine, homonaphthylalanine, bis(homophenylalanine), bis-(homonapbthylalanine), tryptophan, or tyrosine, each of which is optionally substituted with one or more substituents. The amino acid residue having a side chain comprising an aryl group can each independently be a residue of tyrosine, phenylalanine; 1-naphthylalanine, 2-naphthylalanine; tryptophan; phenylglycine; homophenylalanine; or β-homophenylalanine. The amino acid residue having a side chain comprising an aryl group can each independently be a residue of phenylalanine, 2-naphthylalanine, homophenylalanine, β-homophenylalanine or homonaphthylalanine, each of which is optionally substituted with one or more substituents. The amino acid residue having a side chain comprising an aryl group can each be independently a residue of phenylalanine, 2-naphthylalanine, homophenylalanine, or β-homophenylalanine, each of which is optionally substituted with one or more substituents. The amino acid residue having a side chain comprising an aryl group can each be independently a residue of phenylalanine or 2-naphthylalanine, each of which is optionally substituted with one or more substituents. At least one amino acid residue having a side chain comprising an aryl group can be a residue of phenylalanine. Two amino acid residues having a side chain comprising an aryl group can be residues of phenylalanine. Each amino acid residue having a side chain comprising an aryl group can be a residue of phenylalanine.

In embodiments, none of the amino acids having the side chain comprising the aryl or heteroaryl group are contiguous. In embodiments, two amino acids having the side chain comprising the aryl or heteroaryl group can be contiguous. In embodiments, two contiguous amino acids can have opposite stereochemistry. In embodiments, the two contiguous amino acids can have the same stereochemistry. In embodiments, three amino acids having the side chain comprising the aryl or heteroaryl group can be contiguous. In embodiments, three contiguous amino acids can have the same stereochemistry. In embodiments, three contiguous amino acids can have alternating stereochemistry.

The amino acid residues comprising aryl or heteroaryl groups can be L-amino acids. The amino acid residues comprising aryl or heteroaryl groups can be D-amino acids. The amino acid residues comprising aryl or heteroaryl groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

### Amino Acid Residues Having a Side Chain Comprising a Guanidine Group, Guanidine Replacement Group, or Protonated Form Thereof

As used herein, guanidine refers to the structure:

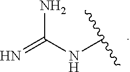 [SMILES: *NC(=N)N]

As used herein, a protonated form of guanidine refers to the structure:

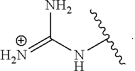 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups may facilitate cell penetration and delivery of a therapeutic agent while reducing toxicity associated with guanidine groups or protonated forms thereof. The cCPP can comprise at least one amino acid having a side chain comprising a guanidine or guanidinium replacement group. The cCPP can comprise two amino acids having a side chain comprising a guanidine or guanidinium replacement group. The cCPP can comprise three amino acids having a side chain comprising a guanidine or guanidinium replacement group. The cCPP can comprise four amino acids having a side chain comprising a guanidine or guanidinium replacement group. The cCPP can comprise five amino acids having a side chain comprising a guanidine or guanidinium replacement group. The cCPP can comprise six amino acids having a side chain comprising a guanidine or guanidinium replacement group.

The guanidine or guanidinium group can be an isostere of guanidine or guanidinium. The guanidine or guanidinium replacement group can be less basic than guanidine.

As used herein, a guanidine replacement group refers to

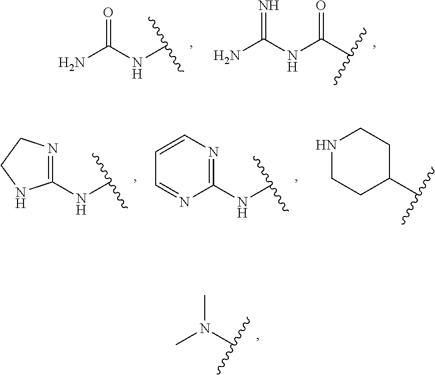

or a protonated form thereof.

In embodiments, a cCPP comprising from 6 to 20 amino acids residues is provided, wherein: (i) at least two amino acids have a side chain comprising a guanidine group, or a protonated form thereof; (ii) at least two amino acids have no side chain or a side chain comprising

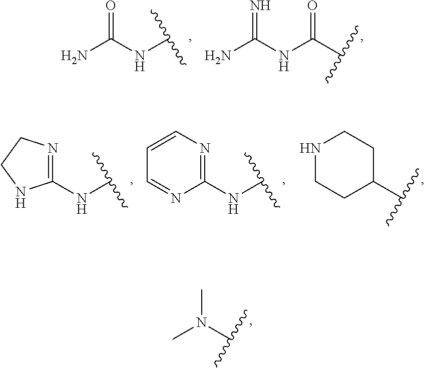

or a protonated form thereof, and (iii) at least two amino acids residues independently have a side chain comprising an aryl or heteroaryl group.

In embodiments, at least two amino acids residues can have no side chain or a side chain comprising

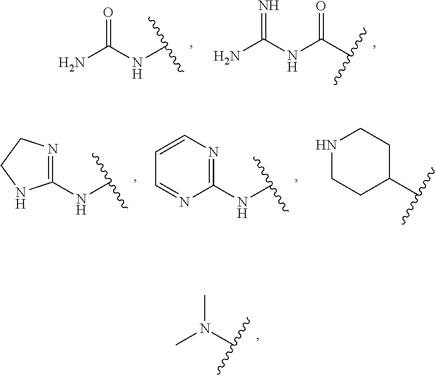

or a protonated form thereof. As used herein, when no side chain is present, the amino acid residue have two hydrogen atoms on the carbon atom(s) (e.g., —CH₂—) linking the amine and carboxylic acid. Glycine or b-alanine are examples of amino acids with no side chain.

In embodiments, the cCPP can comprise at least one amino acid having a side chain comprising one of the following moieties:

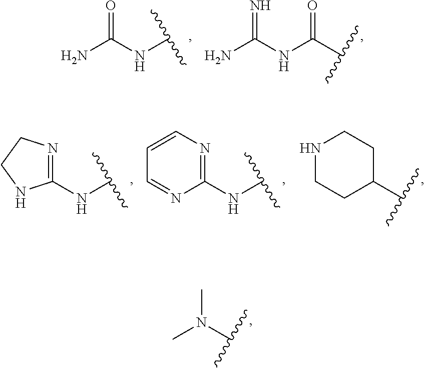

or a protonated form thereof.

In embodiments, the cCPP can comprise at least two amino acids each independently having one of the following moieties

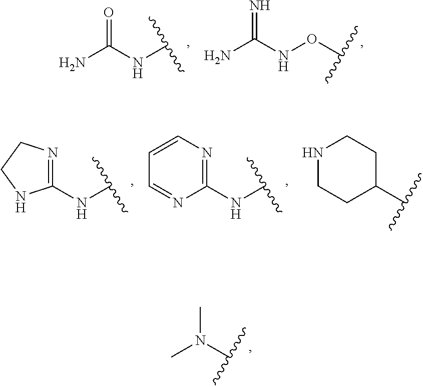

or a protonated form thereof. In embodiments, at least two amino acids can have a side chain comprising the same moiety selected from:

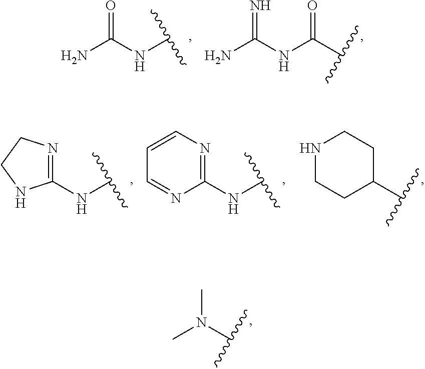

or a protonated form thereof. In embodiments, at least one amino acid can have a side chain comprising

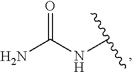 [SMILES: *NC(N)=O]

or a protonated form thereof In embodiments, at least two amino acids can have a side chain comprising

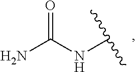 [SMILES: *NC(N)=O]

or a protonated form thereof. In embodiments, one, two, three, or four amino acids can have a side chain comprising

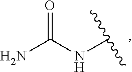 [SMILES: *NC(N)=O]

or a protonated form thereof. In embodiments, one amino acid can have a side chain comprising

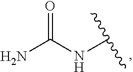 [SMILES: *NC(N)=O]

or a protonated form thereof. In embodiments, two amino acids can have a side chain comprising

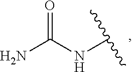 [SMILES: *NC(N)=O]

or a protonated form thereof.

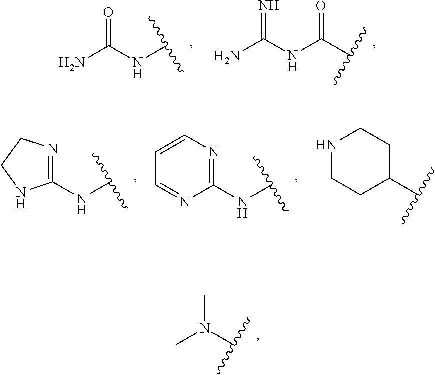

or a protonated form thereof, can be attached to the terminus of the amino acid side chain.

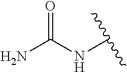 [SMILES: *NC(N)=O]

can be attached to the terminus of the amino acid side chain.

In embodiments, the cCPP can comprise (iii) 2, 3, 4, 5 or 6 amino acid residues independently having a side chain comprising a guanidine group, guanidine replacement group, or a protonated form thereof. In embodiments, the cCPP can comprise (iii) 2 amino acid residues independently having a side chain comprising a guanidine group, guanidine replacement group, or a protonated form thereof. In embodiments, the cCPP can comprise (iii) 3 amino acid residues independently having a side chain comprising a guanidine group, guanidine replacement group, or a protonated form thereof. In embodiments, the cCPP can comprise (iii) 4 amino acid residues independently having a side chain comprising a guanidine group, guanidine replacement group, or a protonated form thereof. In embodiments, the cCPP can comprise (iii) 5 amino acid residues independently having a side chain comprising a guanidine group, guanidine replacement group, or a protonated form thereof. In embodiments, the cCPP can comprise (iii) 6 amino acid residues independently having a side chain comprising a guanidine group, guanidine replacement group, or a protonated form thereof. In embodiments, the cCPP can comprise (iii) 2, 3, 4, or 5 amino acid residues independently having a side chain comprising a guanidine group, guanidine replacement group, or a protonated form thereof. In embodiments, the cCPP can comprise (iii) 2, 3, or 4 amino acid residues independently having a side chain comprising a guanidine group, guanidine replacement group, or a protonated form thereof. In embodiments, the cCPP can comprise (iii) 2 or 3 amino acid residues independently having a side chain comprising a guanidine group, guanidine replacement group, or a protonated form thereof.

In embodiments, the amino acid residues can independently have a side chain comprising the guanidine group, guanidine replacement group, or the protonated form thereof that are not contiguous. In embodiments, two amino acid residues can independently have the side chain comprising the guanidine group, guanidine replacement group, or the protonated form thereof can be contiguous. In embodiments, three amino acid residues can independently have the side chain comprising the guanidine group, guanidine replacement group, or the protonated form thereof can be contiguous. In embodiments, four amino acid residues can independently have the side chain comprising the guanidine group, guanidine replacement group, or the protonated form thereof can be contiguous. In embodiments, the contiguous amino acid residues can have the same stereochemistry. In embodiments, the contiguous amino acids can have alternating stereochemistry.

In embodiments, the amino acid residues independently having the side chain comprising the guanidine group, guanidine replacement group, or the protonated form thereof, can be L-amino acids. In embodiments, the amino acid residues independently having the side chain comprising the guanidine group, guanidine replacement group, or the protonated form thereof, can be D-amino acids. In embodiments, the amino acid residues independently having the side chain comprising the guanidine group, guanidine replacement group, or the protonated form thereof, can be a mixture of L- or D-amino acids.

In embodiments, each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. In embodiments, each amino acid residue having the side chain comprising the guanidine group, or the protonated form thereof, can independently be a residue of arginine or a protonated form thereof.

In embodiments, each amino acid having the side chain comprising a guanidine replacement group, or protonated form thereof, can independently be

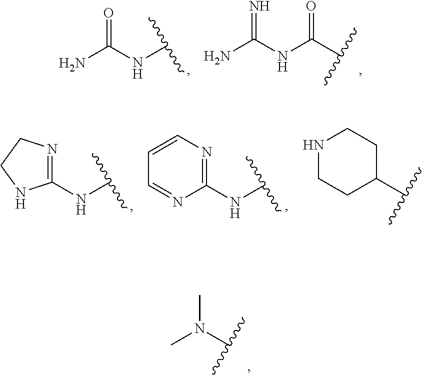

or a protonated form thereof. Those skilled in the art will appreciate that the N- and/or C-termini of the above non-natural aromatic hydrophobic amino acids, upon incorporation into the peptides, form amide bonds.

In embodiments, the cCPP can comprise a first amino acid having a side chain comprising an aryl or heteroaryl group and a second amino acid having a side chain comprising an aryl or heteroaryl group, wherein an N-terminus of a first glycine forms a peptide bond with the first amino acid having the side chain comprising the aryl or heteroaryl group, and a C-terminus of the first glycine forms a peptide bond with the second amino acid having the side chain comprising the aryl or heteroaryl group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may not refer to an amino acid located at the N-terminus of the peptide sequence.

In embodiments, the cCPP can comprise an N-terminus of a second glycine forms a peptide bond with an amino acid having a side chain comprising an aryl or heteroaryl group, and a C-terminus of the second glycine forms a peptide bond with an amino acid having a side chain comprising a guanidine group, or a protonated form thereof.

In embodiments, the cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

In embodiments, the cCPP can comprise a residue of asparagine, aspartic acid, glutamine, glutamic acid, or homoglutamine. In embodiments, the cCPP can comprise a residue of asparagine. In embodiments, the cCPP can comprise a residue of glutamine.

In embodiments, the cCPP can comprise a residue of tyrosine; phenylalanine; 1-naphthylalanine; 2-naphthylalanine; tryptophan; homophenylalanine; or β-homophenylalanine.

While not wishing to be bound by theory, it is believed that the chirality of the amino acids in the cCPPs may impact cytosolic uptake efficiency. In embodiments, the cCPP can comprise at least one D amino acid. In embodiments, the cCPP can comprise one to fifteen D amino acids. In embodiments, the cCPP can comprise one to ten D amino acids. In embodiments, the cCPP can comprise 1, 2, 3, 4, 5, 6, 7 or 8 D amino acids. In embodiments, the cCPP can comprise at least one L amino acid. In embodiments, the cCPP can comprise one to fifteen L amino acids. In embodiments, the cCPP can comprise one to ten L amino acids. In embodiments, the cCPP can comprise 1, 2, 3, 4, 5, 6, 7 or 8 L amino acids. In embodiments, the cCPP can comprise 2, 3, 4, 5, 6, 7, or 8 contiguous amino acids having alternating D and L chirality. In embodiments, the cCPP can comprise three contiguous amino acids having the same chirality. In embodiments, the cCPP can comprise two contiguous amino acids having the same chirality. In embodiments, at least two of the amino acids can have the opposite chirality. In embodiments, the at least two amino acids having the opposite chirality can be adjacent to each other. In embodiments, at least three amino acids can have alternating stereochemistry relative to each other. In embodiments, the at least three amino acids having the alternating chirality relative to each other can be adjacent to each other. In embodiments, at least four amino acids have alternating stereochemistry relative to each other. In embodiments, the at least four amino acids having the alternating chirality relative to each other can be adjacent to each other. In embodiments, at least two of the amino acids can have the same chirality. In embodiments, at least two amino acids having the same chirality can be adjacent to each other. In embodiments, at least two amino acids have the same chirality and at least two amino acids have the opposite chirality. In embodiments, the at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. In embodiments, the amino acid residues that form the cCPP can all be L-amino acids. In embodiments, the amino acid residues that form the cCPP can all be D-amino acids.

In embodiments, at least two of the amino acids can have a different chirality. In embodiments, at least two amino acids having a different chirality can be adjacent to each other. In embodiments, at least three amino acids can have different chirality relative to an adjacent amino acid. In embodiments, at least four amino acids can have different chirality relative to an adjacent amino acid. In embodiments, at least two amino acids have the same chirality and at least two amino acids have a different chirality. In embodiments, one or more amino acid residues that form the cCPP can be achiral. In embodiments, the cCPP can comprise a motif of 3, 4, or 5 amino acids, wherein two amino acids having the same chirality can be separated by an achiral amino acid. The cCPPs can comprise the following sequences: D/L-X-D/L; D/L-X-D/L-X; D/L-X-D/L-X-D/L; D-X-D; D-X-D-X; D-X-D-X-D; L-X-L; L-X-L-X; or L-X-L-X-L, wherein D/L indicates that the amino acid can be a D or an L amino acid and X is an achiral amino acid. The achiral amino acid can be glycine.

In embodiments, an amino acid having a side chain comprising:

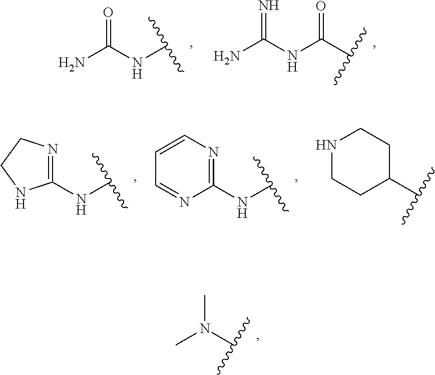

or a protonated form thereof, can be adjacent to an amino acid having a side chain comprising an aryl or heteroaryl group. In embodiments, an amino acid having a side chain comprising:

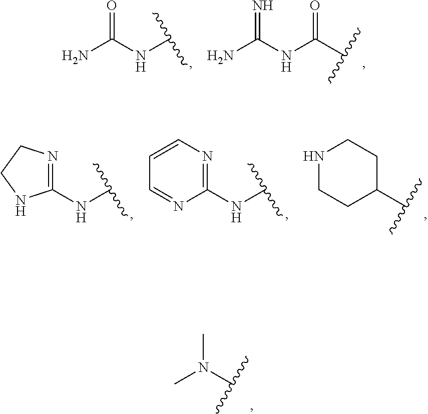

or a protonated form thereof, can be adjacent to at least one amino acid having a side chain comprising a guanidine or protonated form thereof. In embodiments, an amino acid having a side chain comprising a guanidine or protonated form thereof can be adjacent to an amino acid having a side chain comprising an aryl or heteroaryl group. In embodiments, two amino acids having a side chain comprising:

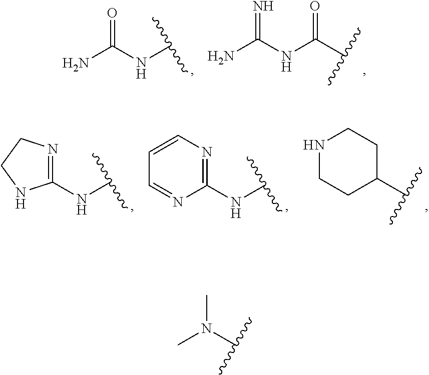

or protonated forms there, can be adjacent to each other. In embodiments, two amino acids having a side chain comprising a guanidine or protonated form thereof are adjacent to each other. In embodiments, a cCPP can comprise at least two contiguous amino acids having a side chain can comprise an aryl or heteroaryl group and at least two non-adjacent amino acids having a side chain comprising:

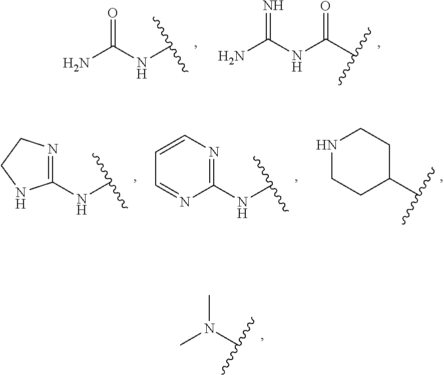

or a protonated form thereof. In embodiments, a cCPP can comprise at least two contiguous amino acids having a side chain comprising an aryl or heteroaryl group and at least two non-adjacent amino acids having a side chain comprising

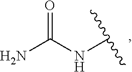 [SMILES: *NC(N)=O]

or a protonated form thereof. In embodiments, the adjacent amino acids can have the same chirality. In embodiments, the adjacent amino acids can have the opposite chirality. Other combinations of amino acids can have any arrangement of D and L amino acids, e.g., any of the sequences described in the preceding paragraph.

In embodiments, at least two amino acids having a side chain comprising:

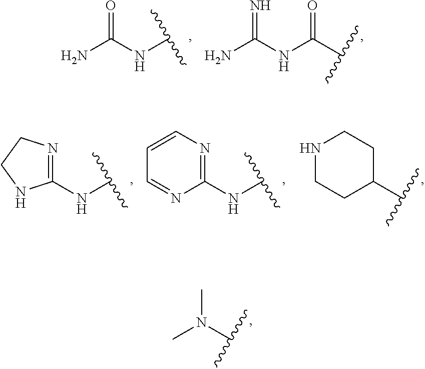

or a protonated form thereof, are alternating with at least two amino acids having a side chain comprising a guanidine group or protonated form thereof.

In embodiments, the cCPP can comprise the structure of Formula (A):

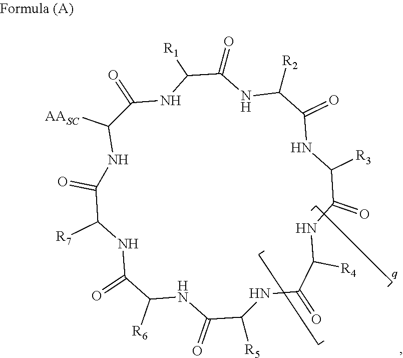

or a protonated form thereof,
wherein:
R₁, R₂, R₃, R₄, R₅, R₆, and R₇ are independently H or an amino acid side chain;
AA_{SC} is an amino acid side chain; and
q is 1, 2, 3 or 4.

The cCPP of the general Formula (A) can have any configuration and/or amino acid side chain as described in the published PCT application NO. US2020/066459 (WO2021127650A1) or U.S. Pat. No. 11,225,506.

In embodiments, AA_{SC} can be

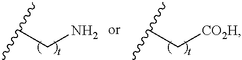

wherein t can be an integer from 0 to 5. AA_{SC} can be

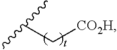

wherein t can be 0 or an integer from 1 to 5. t can be 1 to 5. T is 2 or 3. t can be 2. t can be 3. In embodiments, AA_{SC} can be conjugated to a linker.

In embodiments, the cCPP are of the general Formula (A) or a protonated form thereof, wherein:
R₁, R₂, and R₅ are each independently H or an aryl or heteroaryl side chain of an amino acid;
at least two of R₁, R₂, and R₃ is an aryl or heteroaryl side chain of an amino acid;
R₄, R₅, R₆, R₇ are independently H or an amino acid side chain;
at least two of R₄, R₅, R₆, R₇ is H or a side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, serine, histidine, N,N-dimethyllysine, β-homoarginine, 3-(1-piperidinyl) alanine;
AA_{SC} is an amino acid side chain, and
q is 1, 2, 3 or 4.

In embodiments the cCPP is of Formula (A), where at least one of R₄, R₅, R₆, R₇ are independently an uncharged, non-aromatic side chain of an amino acid. In embodiments, at least two of R₄, R₅, R₆, R₇ are, independently, H or a side chain of serine, histidine, or citrulline.

In embodiments, compounds are provided that include a cyclic peptide having from 6 to 12 amino acids, wherein at least two amino acids of the cyclic peptide are charged amino acids; at least two amino acids of the cyclic peptide are aromatic hydrophobic amino acids; and at least two amino acids of the cyclic peptide are uncharged, non-aromatic amino acids. In embodiments, at least two charged amino acids of the cyclic peptide are arginine. In embodiments, at least two aromatic, hydrophobic amino acids of the cyclic peptide are phenylalanine, naphtha alanine (3-Naphth-2-yl-alanine), or a combination thereof. In embodiments, at least two uncharged, non-aromatic amino acids of the cyclic peptide are citrulline, glycine or a combination thereof. In embodiments, the compound is a cyclic peptide having from 6 to 12 amino acids wherein two amino acids of the cyclic peptide are arginine; at least two amino acids are aromatic, hydrophobic amino acids selected from phenylalanine, naphtha alanine, homophenylalanine and combinations thereof; and at least two amino acids are uncharged, non-aromatic amino acids selected from citrulline, serine, histidine, glycine, and combinations thereof. In embodiments, the compound is a cyclic peptide having from 6 to 12 amino acids wherein two amino acids of the cyclic peptide are arginine; two or three amino acids are, independently, aromatic, hydrophobic amino acids selected from phenylalanine, 2-naphthylalanine, β-homophenylalanine; and two amino acids are independently, uncharged, non-aromatic amino acids selected from citrulline, serine, histidine, and glycine.

The cCPP can comprise the structure of Formula (I):

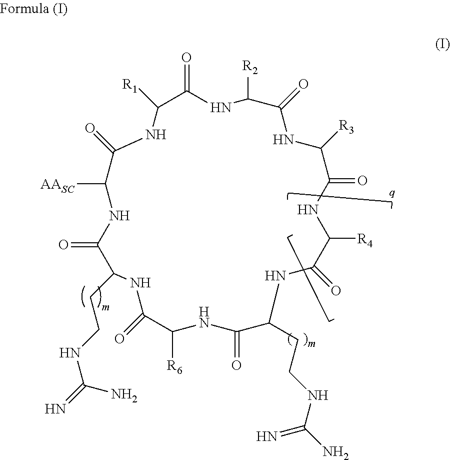

or a protonated form thereof,
wherein:
R₁, R₂, and R₃ can each independently be H or an amino acid residue having a side chain comprising an aryl or heteroaryl group;
at least two of R₁, R₂, and R₃ is an aryl or heteroaryl side chain of an amino acid;
R₄ and R₆ are independently H or an amino acid side chain;
AA_{SC} is an amino acid side chain;
q is 1, 2, 3 or 4; and
each m is independently an integer 0, 1, 2, or 3.

A cCPP of Formula (A) may be of Formula (I).

In embodiments, the cCPP are of Formula (I) or (A), where R₁, R₂, and R₃ can each independently be H; -alkylene-aryl; or -alkylene-heteroaryl. R₁, R₂, and R₃ can each independently be H, —C₁₋₃alkylene-aryl or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂, and R₃ can each independently be H, —CH2Ph, or —CH₂Naphthyl. R₁, R₂, and R₃ can each independently be H or —CH₂Ph.

In embodiments, the cCPP are of Formula (I) or (A), where R₁, R₂, and R₃ can each independently be the side chain of tyrosine, phenylalanine; 1-naphthylalanine; 2-naphthylalanine; tryptophan; 3-benzothienylalanine, 4-phenylphenylalanine; 3,4-difluorophenylalanine; 4-trifluoromethylphenylalanine; 2,3,4,5,6-pentafluorophenylalanine; homophenylalanine; β-homophenylalanine; 4-tert-butyl-phenylalanine; 4-pyridinylalanine; 3-pyridinylalanine; 4-methylphenylalanine; 4-fluorophenylalanine; 4-chlorophenylalanine; or 3-(9-anthryl)-alanine.

In embodiments, the cCPP are of Formula (I) or (A), where R₁ can be the side chain of tyrosine. R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of homophenylalanine. R₁ can be H.

In embodiments, the cCPP are of Formula (I) or (A), where Rz can be the side chain of tyrosine. R₂ can be the side chain of phenylalanine. R₂ can be the side chain of 1-naphthylalanine. R₂ can be the side chain of 2-naphthylalanine. R₂ can be the side chain of tryptophan. R₂ can be the side chain of homophenylalanine. R₂ can be H.

In embodiments, the cCPP are of Formula (I) or (A), where R₃ can be the side chain of tyrosine. R₃ can be the side chain of phenylalanine. R₃ can be the side chain of 1-naphthylalanine. R₃ can be the side chain of 2-naphthylalanine. R₃ can be the side chain of tryptophan. R₃ can be the side chain of homophenylalanine. R₃ can be H.

In embodiments, the cCPP are of Formula (I) or (A), where R₄ can be H, or a side chain of arginine, citrulline, serine or histidine. R₄ can be H. R₄ can be a side chain of arginine. R₄ can be a side chain of citrulline. R₄ can be a side chain of serine. R₄ can be a side chain of histidine.

In embodiments, the cCPP are of Formula (A), where R₅ can be H, or a side chain of arginine, citrulline, serine or histidine. R₅ can be H. R₅ can be a side chain of arginine. R₅ can be a side chain of citrulline. R₅ can be a side chain of serine. R₅ can be a side chain of histidine.

In embodiments, the cCPP are of Formula (I) or (A), where R₆ can be H, or a side chain of arginine, citrulline, serine or histidine. R₆ can be H. R₆ can be a side chain of arginine. R₆ can be a side chain of citrulline. R₆ can be a side chain of serine. R₆ can be a side chain of histidine.

In embodiments, the cCPP are of Formula (A), where R₇ can be H, or a side chain of arginine, citrulline, serine or histidine. R₇ can be H. R₇ can be a side chain of arginine. R₇ can be a side chain of citrulline. R₇ can be a side chain of serine. R₇ can be a side chain of histidine.

In embodiments, the cCPP are of Formula (I) or (A), where one, two, or three of R₁, R₂, R₃, R₄, R₅, R₆, and R₇ can be H. One of R₁, R₂, R₃, R₄, R₅, R₆, and R₇ can be H. Two of R₁, R₂, R₃, R₄, R₅, R₆, and R₇ can be H. Three of R₁, R₂, R₃, R₅, R₆, and R₇ can be H. At least one of R₁, R₂, R₃, R₄, R₅, R₆, and R₇ can be H.

In embodiments, the cCPP are of Formula (I) or (A), where one of R₁, R₂, or R₃, is H.

In embodiments, the cCPP are of Formula (I) or (A), where at least one of R₄, R₅, R₆, and R₇ can be H or a side chain of arginine, citrulline, serine, or histidine. At least one of R₄, R₅, R₆, and R₇ is H. At least one of R₄, R₅, R₆, and R₇ can be side chain of arginine. At least one of R₄, R₅, R₆, and R₇ can be side chain of citrulline. At least one of R₄, R₅, R₆, and R₇ can be side chain of serine. At least one of R₄, R₅, R₆, and R₇ can be side chain of histidine. One of R₄, R₅, R₆, and R₇ can be H or a side chain of arginine, citrulline, serine, or histidine. One of R₄, R₅, R₆, and R₇ is H. One of R₄, R₅, R₆, and R₇ can be side chain of arginine. One of R₄, R₅, R₆, and R₇ can be side chain of citrulline. One of R₄, R₅, R₆, and R₇ can be side chain of serine. One of R₄, R₅, R₆, and R₇ can be side chain of histidine.

In embodiments, the cCPP are of Formula (I) or (A), where two of R₄, R₅, R₆, and R₇ can be H or a side chain of arginine, citrulline, serine, or histidine. Two of R₄, R₅, R₆, and R₇ can be H. Two of R₄, R₅, R₆, and R₇ can be side chain of arginine. Two of R₄, R₅, R₆, and R₇ can be side chain of citrulline. Two of R₄, R₅, R₆, and R₇ can be side chain of serine. Two of R₄, R₅, R₆, and R₇ can be side chain of histidine.

In embodiments, the cCPP are of Formula (A), where three of R₄, R₅, R₆, and R₇ can be H or a side chain arginine, citrulline, serine or histidine. Three of R₄, R₅, R₆, and R₇ can be H. Three of R₄, R₅, R₆, and R₇ can be side chain of arginine. Three of R₄, R₅, R₆, and R₇ can be side chain of citrulline. Three of R₄, R₅, R₆, and R₇ can be side chain of serine. Three of R₄, R₅, R₆, and R₇ can be side chain of histidine.

In embodiments, the cCPP are of Formula (A), where AA_{SC} can be a side chain of a residue of asparagine, glutamine, or homoglutamine. AA_{SC} can be a side chain of a residue of glutamine. The cCPP can further comprise a linker conjugated to the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

In embodiments, the cCPP are of Formula (A), where q can be 1, 2, or 3. q can 1 or 2. q can be 1. q can be 2. q can be 3. q can be 4.

In embodiments, the cCPP are of Formula (A), where m can be an integer from 1 to 3. m can be 1 or 2. m can be 0. m can be 1. m can be 2. m can be 3.

The cCPP of Formula (A) or (I) can comprise the structure of Formula (I-a) or Formula (I-b):

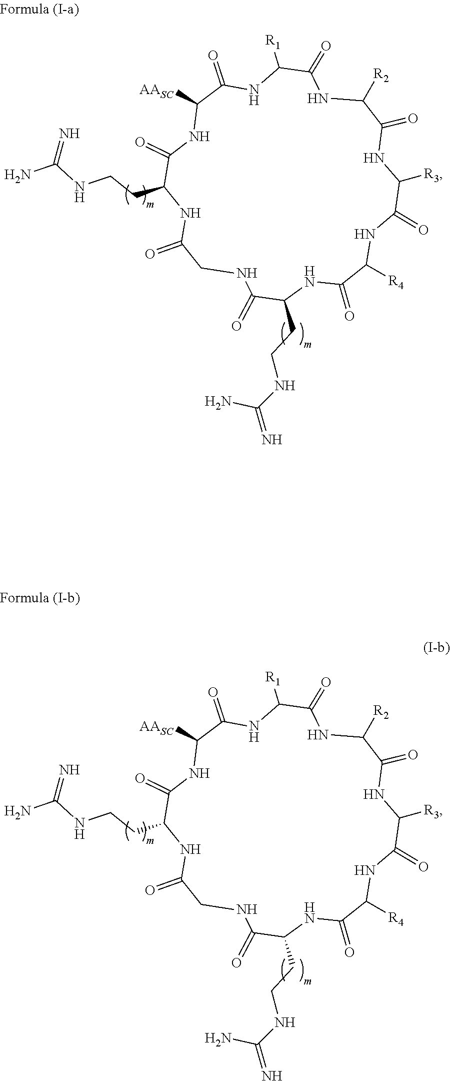

or protonated form thereof, wherein AA_{SC}, R₁, R₂, R₃, R₄, and m are as defined herein relative to Formula (A) and/or Formula (I).

The cCPP of Formula (A) or (I) can comprise the structures of Formulae (I-1), (I-2), (I-3), (I-4), (1-5), (I-6) or (I-7):

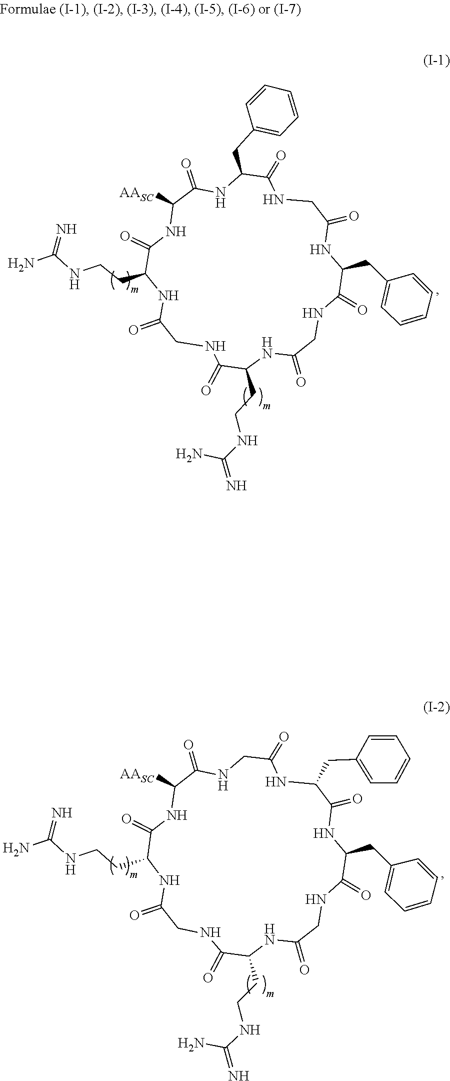

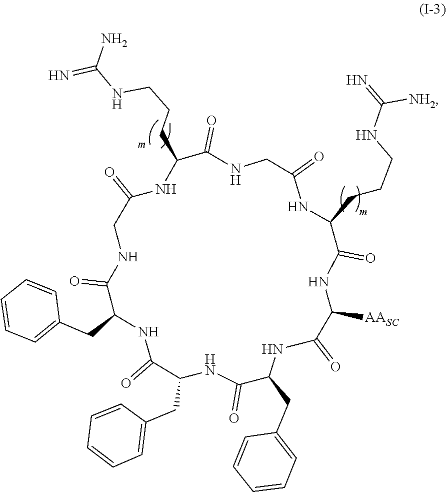

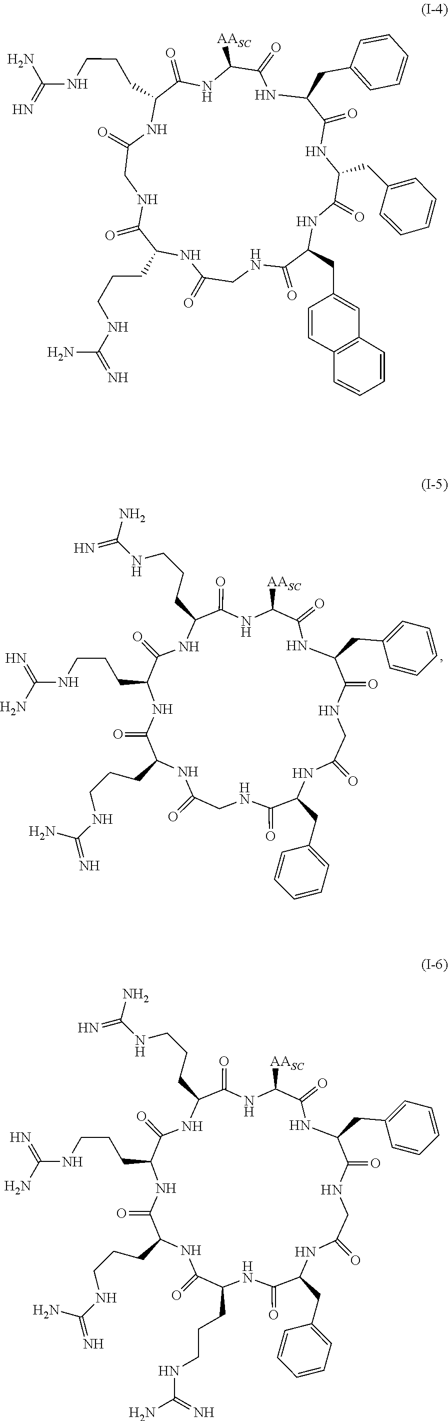

or protonated form thereof, wherein AA_{SC} and m are as defined herein relative to Formula (A) and/or Formula (I).

The cCPP can comprise one of the following sequences: FfΦRrRr, FGFGRGR; GfFGrGr, FfΦGRGR; FfFGRGR; FfΦGrGr; FGFGRRR; or FGFRRRR. The cCPP can have one of the following sequences: FfΦRrRrQ, FGFGRGRQ; GfFGrGrQ, FfΦGRGRQ; FfFGRGRQ; FfΦGrGrQ; FGFGRRRQ; or FGFRRRRQ.

The cCPP of Formula (A) or Formula (I) can have the structure of Formula (I-c):

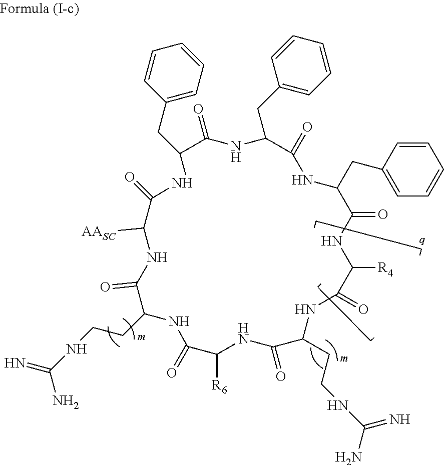

or a protonated form thereof, wherein R₄, R₆, q, m and AA_{SC} are as defined herein.

The cCPP of Formula (IA) or Formula (I) can have the structure of Formula (I-d):

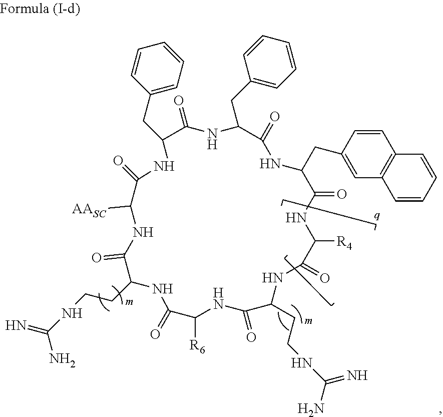

or a protonated form thereof, wherein R₄, R₆, q, m, and AA_{SC} are as defined herein.

The cCPP of Formula (A) or Formula (I) can have the structure of Formula (I-e):

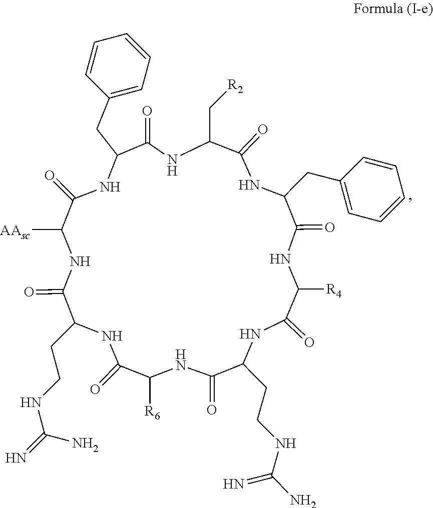

or a protonated form thereof,
wherein: R₂, R₄, R₆, q, m, and AA_{SC} are as defined herein. In embodiments, R₂ is H. The cCPP of Formula (A) can be selected from:

CPP Sequence

(FfΦRrRrQ)
(FfΦCit-r-Cit-rQ)
(FfΦGrGrQ)

The cCPP of Formula (A) can be selected from:

(FfFGRGRQ)
(FGFGRGRQ)
(GfFGrGrQ)
(FGFGRRRQ)
(FGFRRRRQ)

The cCPP of Formula (A) can be selected from:

FfΦGRGRQ
βhF-FΦSRSRQ
βhF-FΦGRGRQ

In embodiments, the cCPP is selected from:

CPP sequence
FΦRRRQ
FΦRRRC
FΦRRRU
RRRΦFQ
RRRRΦF
FΦRRRR
FφrRrRq
FφrRrRQ
FΦRRRRQ
fΦRrRrQ
RRFRΦRQ
FRRRRΦQ
rRFRΦRQ
RRΦFRRQ
CRRRRFWQ
FfΦRrRrQ
FFΦRRRRQ
RFRFRΦRQ
URRRRFWQ
RRFRΦRQ
FRRRRΦQ
CRRRRFWQ
FΦRRRRQK
FΦRRRRQC
fΦRrRrRQ
FΦRRRRRQ
RRRRΦFDΩC
FΦRRR
FWRRR
RRRΦF
RRRWF

Where Φ = L-naphthylalanine; φ = D-naphthylalanine; Ω = L-norleucine

In embodiments, the cCPP can be of the Formula (II)

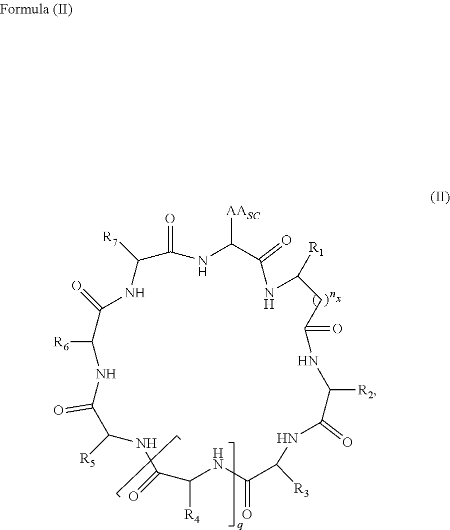

or a protonated form thereof,
wherein:
R₁, R₂, and R₃ can each independently be H or an amino acid residue having a side chain comprising an aryl or heteroaryl group;
at least two of R₁, R₂, and R₃ is an aryl or heteroaryl side chain of an amino acid;
R₄, R₅, R₆, R₇ are independently H or an amino acid side chain;
at least two of R₄, R₅, R₆, R₇ are independently a side chain of arginine;
AA_{SC} is an amino acid side chain;
nₓ is 0 or 1; and
q is 1, 2, 3 or 4.

In embodiments, the cCPP can be of the Formula (III)

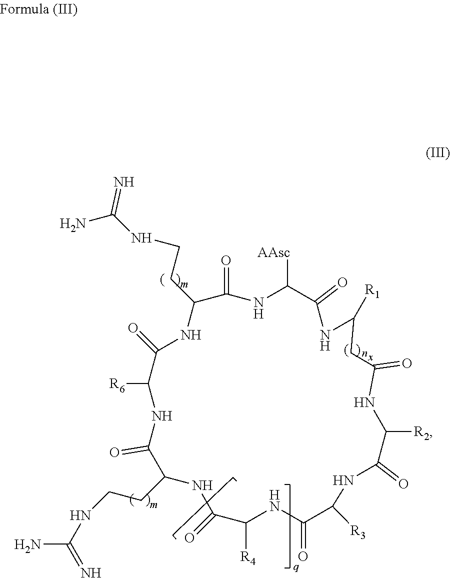

or a protonated form thereof, wherein;
R₁, R₂, and R₃ can each independently be H or an amino acid residue having a side chain comprising an aryl or heteroaryl group;
at least two of R₁, R₂, and R₃ is an aryl or heteroaryl side chain of an amino acid;
R₄, and R₆ are independently H or an amino acid side chain;
AA_{SC} is an amino acid side chain;
nₓ is 0 or 1;
each m is independently an integer 0, 1, 2, or 3; and
q is 1, 2, 3 or 4.

In embodiments, the cCPP is of Formula (II) or (III) where at least one of R₁, R₂, or R₃ are, independently, H. In embodiments, the cCPP is of Formula (II) or Formula (III) where at least one of R₁, R₂, and R₃ are, independently, an amino acid residue having a side chain comprising an aryl or heteroaryl group. In embodiments, the amino acid residue having a side chain comprising an aryl or heteroaryl group is phenylalanine, beta homophenylalanine, or 3-(2-naphthyl)-alanine. In embodiments, the cCPP is of Formula (II) where at least two of R₄, R₆ are each independently an amino acid residue having a side chain comprising a charged group. In embodiments, the amino acid residue having a side chain comprising a charged group is arginine. In embodiments, the cCPP is of Formula (II) or (III) where q is 1.

In embodiments, the cCPP is of Formula (II) where nₓ is 1 and where R₅ and R₇ are a side chain of arginine. In embodiments, the cCPP is of Formula (II) where nₓ is 1, wherein the aryl or heteroaryl group is phenylalanine, beta homophenylalanine, or 3-(2-naphthyl)-alanine, and where at least two of R₄, R₅, R₆ and R₇ are the side chain of arginine. In embodiments, the cCPP is of Formula (II) where nₓ is 1, where R₄, R₅, R₆ and R₇ are the side chain of arginine. In embodiments, the cCPP is of Formula (II) where nₓ is 1, where R₅ and R₇ are the side chain of arginine, and R₄ and R₆ are H. In embodiments, the cCPP is of Formula (II) or (III) where at least one of R₄, R₅ (if present), R₆, or R₇ (if present) are H or the amino acid side chain of serine or histidine. In embodiments the cCPP is of Formula (II) or (III) where at least two of R₄, R₅ (if present), R₆, or R₇ (if present) are, independently, H or the amino acid side chain of serine or histidine. In embodiments the cCPP is of Formula (II) where at least three of R₄, R₅, R₆, or R₇ are, independently, H or the amino acid side chain of serine or histidine. In embodiments the cCPP is of Formula (II) where at least four of R₄, R₅, R₆, or R₇ are, independently, H or the amino acid side chain of serine or histidine.

In embodiments of the cCPP of Formula (II): at least two of R₁, R₂, and R₃ are independently a side chain of phenylalanine, betahomophenylalanine, or naphthylalanine; at least two of R₄, R₅, R₆, or R₇ are independently a side chain of arginine; at least two of R₄, R₅, R₆, or R₇ are independently H or a side chain of arginine, serine or histidine; AA_{SC} is an amino acid side chain; nₓ is 0 or 1; and q is 1. It is understood that nₓ is 1 when R₁ is a side chain of betahomophenylalanine.

In embodiments of the cCPP of Formula (III): at least two of R₁, R₂, and R₃ are independently a side chain of phenylalanine, or naphthylalanine; R₄ and R₆ are each independently H or a side chain of arginine, serine or histidine; AA_{SC} is an amino acid side chain; nₓ is 0 or 1; and q is 1.

In embodiments, the cCPP is of Formula (II) or (III), where two of R₄, R₅ (if present), R₆, or R₇ (if present) are independently a side chain of serine. In embodiments, the cCPP is of Formula (II) or (III), where two of R₄, R₅ (if present), R₆, or R₇ (if present) are independently a side chain of histidine. In embodiments, the cCPP is of Formula (II) or (III), where two of R₄, R₅ (if present), R₆, or R₇ (if present) are independently, H.

In embodiments, the CPP is of Formula (II) wherein: at least two of R₁, R₂, and R₃ are independently a side chain of phenylalanine, betahomophenylaline, or naphthylalanine; at least two of R₄, R₅, R₆, or R₇ are independently a side chain of arginine; at least two of R₄, R₅, R₆, or R₇ are independently H or a side chain of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine; AA_{SC} is an amino acid side chain; nₓ is 0 or 1; and q is 1. It is understood that nₓ is 1 when R₁ is a side chain of betahomophenylalanine.

In embodiments, the CPP is of Formula (III) wherein: at least two of R₁, R₂, and R₃ are independently a side chain of phenylalanine, naphthylalanine, or betahomophenylanine; R₄ and R₇ are independently H or a side chain of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine; AA_{SC} is an amino acid side chain; nₓ is 0 or 1; and q is 1. It is understood that nₓ is 1 when R₁ is a side chain of betahomophenylalanine.

In embodiments, the CPP is of Formula (II) wherein: at least two of R₁, R₂, and R₃ are independently a side chain of phenylalanine, betahomophenylalanine, or naphthylalanine; at least two of R₄, R₅, R₆, or R₇ are independently a side chain of arginine; at least two of R₄, R₅, R₆, or R₇ are independently a side chain of serine or histidine; AA_{SC} is an amino acid side chain, nₓ is 0 or 1; and q is 1. It is understood that nₓ is 1 when R₁ is a side chain of betahomophenylalanine.

In embodiments, the CPP is of Formula (III) wherein: at least two of R₁, R₂, and R₃ are independently a side chain of phenylalanine, betahomophenylaline, or naphthylalanine; R₄ and R₆ are independently a side chain of serine or histidine; AA_{SC} is an amino acid side chain; nₓ is 0 or 1; and q is 1. It is understood that nₓ is 1 when Riis a side chain of betahomophenylalanine.

In embodiments, the CPP is of the general Formula (II) or (III), wherein at least one of R₁, R₂, or R₃ is H. In embodiments, the CPP is of the general Formula (II) or (III), wherein at least one of R₁, R₂, or R₃ is a side chain of phenylalanine. In embodiments, the CPP is of the general Formula (II) or (III), wherein at least two of R₁, R₂, or R₃ are a side chain of phenylalanine. In embodiments, the CPP is of the general Formula (II) or (III), wherein at least one of R₁, R₂, or R₃ is a side chain of naphtbylalanine.

In embodiments, the CPP is of the general Formula (II), wherein at least two of R₄, R₅, R₆, or R₇ are independently a side chain of serine or histidine. In embodiments, the CPP is of the general Formula (III), wherein R₄, and R₆ are independently H, or a side chain of serine or histidine. In embodiments, the CPP is of the general Formula (III), wherein R₄, and R₆ are the side chain of serine. In embodiments, the CPP is of the general Formula (III), wherein R₄, and R₆ are the side chain of histidine. In embodiments, the CPP is of the general Formula (III), wherein R₄, and R₆ are H.

In embodiments, the CPP is of the general Formula (II) or (III), wherein at least one of R₄, R₅ (if present), R₆, R₇ (if present) are independently an uncharged, non-aryl side chain of an amino acid. In embodiments, the CPP is of the general Formula (II) or (III), wherein at least two of R₄, R₅ (if present), R₆, or R₇ (if present) are independently side chains of an uncharged non-aryl amino acid (e.g., histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine). In embodiments, the CPP is of the general Formula (II) or (III), wherein at least two of R₄, R₅ (if present), R₆, or R₇ (if present) are independently side chains of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine. In embodiments, the CPP is of the general Formula (II) or (III), wherein at least two of R₄, R₅ (if present), R₆, or R₇ (if present) are independently side chains of an uncharged non-aryl amino acid selected from histidine, and serine.

In embodiments, the CPP is of the general Formula (II), wherein at least one of R₄, R₅, R₆, R₇ is, independently, H. In embodiments, the CPP is of the general Formula (II), wherein two of R₄, R₅, R₆, R₇ are, independently, H. In embodiments, the CPP is of the general Formula (III), wherein at least one of R₄ or R₆ is, independently, H. In embodiments, the CPP is of the general Formula (III), wherein two of R₄ and R₆ are H.

In embodiments, compounds are provided that include a cyclic peptide having from 6 to 12 amino acids, wherein at least two amino acids of the cyclic peptide are charged amino acids, at least two amino acids of the cyclic peptide are aryl or heteroaryl hydrophobic amino acids and at least two amino acids of the cyclic peptide are uncharged, non-aryl amino acids. In embodiments, at least two charged amino acids of the cyclic peptide are arginine. In embodiments, at least two aryl or heteroaryl, hydrophobic amino acids of the cyclic peptide are phenylalanine, naphthylalanine (3-naphth-2-yl-alanine), betahomophenylalanine, or a combination thereof. In embodiments, at least two uncharged, non-aryl amino acids of the cyclic peptide are glycine. In embodiments, two of the uncharged amino acids are serine, histidine or a combination thereof.

In embodiments, the CPP) may comprise one of the following sequences: FGFGHGH; FGFSHSH; FGFGHGHQ; or FGFSHSHQ. In embodiments, the cCPP can comprise one of the following sequences: βhF-FΦSRSR, βhF-FΦGRGR, bhF-f-ΦGrGr; bhF-fΦSRSR; or FfΦSrSr. The cCPP can comprise one of the following sequences: βhF-FΦSRSRQ, βhF-FΦGRGRQ, bhF-fΦGrGrQ; bhF-fΦSRSRQ; or FfΦSrSrQ. In embodiments, the cCPP can comprise one of the following sequences: FfFSRSR; FGFSRSR; βhF-f-Nal-SRSR; FfFSRSRQ; FGFSRSRQ; or βhF-f-Nal-SRSRQ.

The cCPP of Formula (AV) or Formula (II) can have the structure of Formula (II-A) or (II-B):

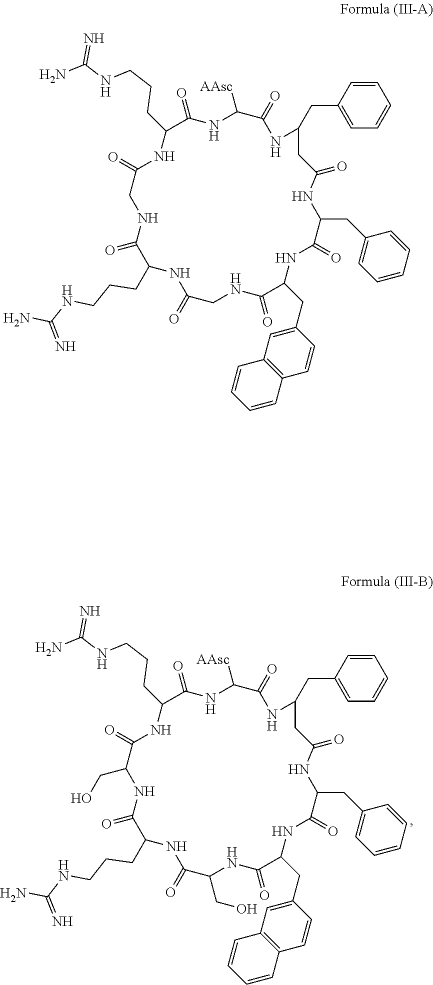

or a protonated form thereof, wherein AA_{SC} is as defined herein.

The cCPP can comprise one of the following sequences: βhF-FΦSRSR, βhF-FΦGRGR, bhF-f-ΦGrGr; bhF-fΦSRSR; or FfΦSrSr. The cCPP can comprise one of the following sequences: βhF-FΦSRSRQ, βhF-FΦGRGRQ, bbF-fΦGrGrQ; bhF-fΦSRSRQ; or FfΦSrSrQ.

In embodiments, the cCPP can comprise one of the following sequences: FfFSRSR; FGFSRSR; βhF-f-Nal-SRSR; FfFSRSRQ; FGFSRSRQ; or βhF-f-Nal-SRSRQ.

In embodiments, AA_{SC} can be

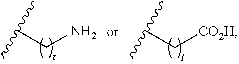

wherein t can be an integer from 0 to 5. AA_{SC} can be

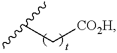

wherein t can be 0 or an integer from 1 to 5. t can be 1 to 5. T is 2 or 3. t can be 2. t can be 3. In embodiments, AA_{SC} can be conjugated to a linker.

The cCPP can be selected from:

CPP Sequence
(bhF-fΦGrGrQ)
(bhF-fΦSrSrQ)
(bhF-fΦHrHrQ)
(bhF-FΦSRSRQ)
(bhF-FΦGRGRQ)
(bhF-FΦHRHRQ)
(FfΦSrSrQ)
(FfΦHrHrQ)

### Exocyclic Peptides

In embodiments, the ocular delivery construct comprises a cyclic cell penetrating peptide (cCPP) and an exocyclic peptide (EP); or an endosomal escape vehicle (EEV) that comprises a cCPP, an EP, and one or more linkers.

The EP can comprise a sequence of a nuclear localization signal (NLS). The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise from 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, alloisoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in Table 2 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2, 3, or 4 lysine residues. The amino group on the side chain of each lysine residue can be substituted with a protecting group, including, for example, trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH, KHKK, KKHK, KKKH, KHKH, HKHK, KKKK, KKRK, KRKK, KRRK, RKKR, RRRR, KGKK, KKGK, HBHBH, HBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH, RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG, wherein B is beta-alanine. The amino acids in the EP can have D or L stereochemistry.

The EP can comprise KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can comprise PKKKRKV, RR, RRR, RHR, RBR, RBRBR, RBHBR, or HBRBH, wherein B is beta-alanine. The EP can comprise PKKKRKV. The amino acids in the EP can have D or L stereochemistry.

The EP can consist of KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can consist of PKKKRKV, RR, RRR, RHR, RBR, RBRBR, RBHBR, or HBRBH, wherein B is beta-alanine. The EP can consist of PKKKRKV. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV. The EP can consist of an NLS comprising the amino acid sequence PKKKRKV. The EP can comprise an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK. The EP can consist of an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK

All exocyclic sequences can also contain an N-terminal acetyl group. Hence, for example, the EP can have the structure: Ac-PKKKRKV.

### Endosomal Escape Vehicles (EEVs)

In embodiments, a cargo conjugate comprising a cargo and an ocular delivery construct is provided. In embodiments, the ocular delivery construct comprises an endosomal escape vehicle (EEV). The EEV can comprise at least one cell penetrating peptide (CPP), for example, a cyclic cell penetrating peptide (cCPP), which is conjugated to an exocyclic peptide (EP). In embodiments, the EEV comprises one cCPP. In embodiments, the EEV comprises one or more linkers.

The configuration of the cargo conjugate comprising an EEV may vary. In embodiments, the EP can be coupled to the cargo. In embodiments, the EP can be coupled to the cCPP. In embodiments, the EP can be coupled to the cargo and the cCPP. In embodiments, the EP can be coupled to the linker. In embodiments, the cCPP can be coupled to the cargo. In embodiments, the cCPP can be coupled to the EP. In embodiments, the cCPP can be coupled to the cargo and the EP. In embodiments, the cCPP can be coupled to the linker. In embodiments, the cargo can be coupled to the EP. In embodiments, the cargo can be coupled to the cCPP. In embodiments, the cargo can be coupled to the cCPP and the EP. In embodiments, the cargo can be coupled to the linker. In embodiments, the cCPP and the EP can be coupled to the linker and the cargo can be coupled to the cCPP or the EP. In embodiments, the cCPP and the cargo can be coupled to the linker and the EP can be coupled to the cCPP or the cargo. In embodiments, the EP and the cargo can be coupled to the linker and the cCPP can be coupled to the EP or the cargo. In embodiments, the cCPP, the EP, and the cargo can be coupled to the linker.

Coupling between the EP, cargo, cCPP, linker, or combinations thereof, may be non-covalent or covalent. In embodiments, the EP can be attached through a peptide bond to the N-terminus of the cCPP. In embodiments, the EP can be attached through a peptide bond to the C-terminus of the cCPP. In embodiments, EP can be attached to the cCPP through a side chain of an amino acid in the cCPP. In embodiments, the EP can be attached to the cCPP through a side chain of a lysine which can be conjugated to the side chain of a glutamine in the cCPP. In embodiments when the cargo is a therapeutic oligonucleotide, the EP can be conjugated to the 5′ or 3′. In embodiments, the EP can be conjugated to an amino group of the linker. In embodiments, the EP can be coupled to a linker via the C-terminus of the EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to conjugate the EP to the cCPP and the C- or N-terminus may be attached to the linker or the cargo.

### Linker

One or more linkers (L) may be used to link the components of a delivery construct and/or the cargo conjugates. A linker is a moiety that covalently couples two or more components of a delivery construct and/or a cargo construct, includes one or more functional group that can be used to conjugate one or more components to a linker; or both. In embodiments, the functional group on the linker may be reacted with a functional group on a component (e.g., an EP, cCPP, or cargo) to form a reaction product and covalently couple to component to the linker and link the component to one or more other components covalently attached to the linker.

The delivery constructs and/or cargo conjugates of the disclosure can include one or more linkers. The linker can link a cargo to the cCPP. The linker can link an EP to the cCPP. The linker can link the cCPP to the EP and the cargo. The linker can be attached to the side chain of an amino acid of the cCPP, and the cargo can be attached at a suitable position on the linker.

The linker can be any appropriate moiety which can conjugate a cCPP to one or more additional components, e.g., an exocyclic peptide (EP) and/or a cargo. Prior to conjugation (e.g., to the cCPP and/or one or more additional components), the linker has two or more functional groups, each of which is independently capable of forming a covalent bond (e.g., to the cCPP and/or one or more additional components). If the cargo is an oligonucleotide, the linker can be covalently bound to the 5′ end of the cargo or the 3′ end of the oligonucleotide cargo. The linker can be covalently bound to the 5′ end of the oligonucleotide cargo. The linker can be covalently bound to the 3′ end of the oligonucleotide cargo. If the cargo is a peptide, the linker can be covalently bound to the N-terminus or the C-terminus of the peptide cargo. The linker can be covalently bound to the backbone (e.g., somewhere in the middle and not at a terminus or termini) of the oligonucleotide or peptide cargo. The linker can be any appropriate moiety that conjugates a cCPP described herein to a therapeutic moiety such as an oligonucleotide, peptide or small molecule.

While not wishing to be bound by theory, the linkers can be chemically functionalized to influence efficacy and tolerability of the resultant construct, for example to increase efficacy. In embodiments, efficacy is increased without decreasing tolerability. For example, the linker can comprise one or more PEG (polyethylene glycol) regions and/or the linker can vary in length. The linker length (e.g., between the EP and cCPP) can influence efficacy and tolerability. In some instances, as linker length decreases, efficacy increases and tolerability decreases; and as linker length increases, efficacy decreases and tolerability increases. In embodiments, as the length of the linker increases (e.g., between a cargo and EEV), the tolerability increases. The linker can comprise hydrocarbon linker.

The linker can comprise a cleavage site. The cleavage site can be a disulfide, or caspase-cleavage site (e.g, Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹-J-R²)z″- subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) —(R¹-J)z″- or -(J-R¹)z″-, wherein each of R¹, at each instance, is independently alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or —(R¹-J-R²)z″-, wherein each of R¹ and R², at each instance, are independently alkylene, each J is independently C, NR³, —NR³C(O)—, S, and O, wherein R⁴ is independently selected from H and alkyl, and z″ is an integer from 1 to 50; or combinations thereof.

The linker can comprise a —(OCH₂CH₂)_{z′}— (e.g., as a spacer), wherein z′ is an integer from 1 to 60, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 29, 40, 41, 42, 43, 44, 45, 46, 47, 48, 49, 50, 51, 52, 53, 54, 55, 56, 57, 58, 59, or 60. “—(OCH₂CH₂)_{z′} can also be referred to as polyethylene glycol (PEG).

The linker can comprise one or more amino acids. The linker can comprise a peptide. The linker can comprise a —(OCH₂CH₂)_{z′}—, wherein z′ is an integer from 1 to 60, and a peptide. The peptide can comprise from 2 to 10 amino acids. The linker can further comprise a functional group (FG) capable of reacting through click chemistry. FG can be an azide or alkyne, and a triazole is formed when the ASO is conjugated to the linker. The linker can further comprise a carboxylic acid functional group capable of reacting with an amine to form an amide when an ASO is conjugated to the linker.

The linker can comprise (i) a β alanine residue and lysine residue; (ii) -(J-R¹)z″; or (iii) a combination thereof. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of β-alanine, glycine, lysine, 4-aminobutyric acid, 5-aminopentanoic acid, 6-aminohexanoic acid or combinations thereof; and (ii) —(R¹ J)z″- or -(J-R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O. The linker can comprise glycine, beta-alanine, 4-aminobutyric acid, 5-aminopentanoic acid, 6-aminohexanoic acid, or a combination thereof.

The linker can be a trivalent linker. The linker can have the structure:

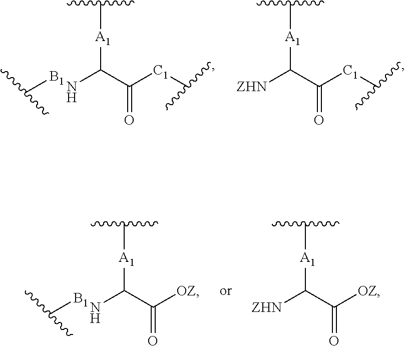

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ₙ—COOH], or caspase-cleavage site (Val-Cit-PABC).

The hydrocarbon can be a residue of glycine or beta-alanine.

The linker can be bivalent and link the cCPP to an AC. The linker can be bivalent and link the cCPP to an exocyclic peptide (EP).

The linker can be trivalent and link the cCPP to an AC and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The cargo can be coupled to the glutamic acid of the cyclic peptide, which converts the glutamic acid to glutamine. The linker (L) can couple the cargo to the glutamine/glutamic acid of the cyclic peptide. In embodiments, a linker (L) is covalently bound to the backbone of the cargo.

The linker can have the structure:

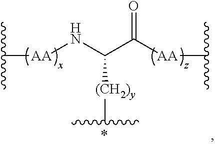

wherein: each AA is independently an amino acid residue; * is the point of attachment to the AA_{SC}, and AA_{SC} is side chain of an amino acid residue of the cCPP; x is an integer from 0-10; y is an integer from 1-5; and z Is an integer from 1-10, x can be an integer from 0-5. x can be an integer from 0-3. X can be 1. x can be 0. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

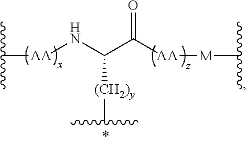

wherein: x is an integer from 0-10; y is an integer from 1-5; z is an integer from 1-10, each AA is independently an amino acid residue; * is the point of attachment to the AA_{SC}, and AA_{SC} is side chain of an amino acid residue of the cCPP; and M is a bonding group defined herein.

In embodiments where a linker has a terminal NH that is conjugated to an exocyclic peptide (EP), one of ordinary skill in the art would understand that the terminal NH of the linker may be a part of the EP. For example, the terminal NH of the linker may be a part of an amide bond formed between the reaction of the carboxylic acid of the C-terminal amino acid of the EP with an amine of the linker. As such, the terminal NH of the linker may be covalently bonded to the carbonyl of the C-terminal amino acid of the EP.

As one of ordinary skill in the art would understand, when an amino acid side chain of the cCPP is covalently attached to the linker, the stated side chain includes a covalent bond between an atom of the side chain and the linker. For example, in embodiments when the linker is covalently attached to the side chain of glutamine, the glutamine side chain includes a covalent bond between the nitrogen of the side chain of glutamine and the linker.

The linker can have the structure:

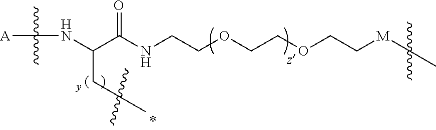

wherein: y is an integer from 1-5; z′ is an integer from 1-60; \hat{ } is the point of attachment to the EP; * is the point of attachment to the AA_{SC}, and AA_{SC} is a side chain of an amino acid residue of the cCPP; and M is a bonding group defined herein.

The linker can have the structure:

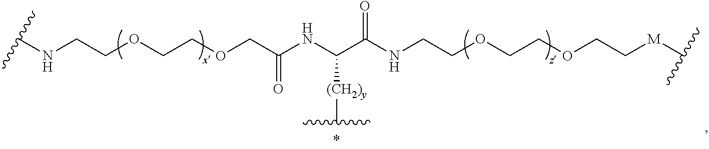

wherein: x′ is an integer from 1-60; y is an integer from 1-5; z′ is an integer from 1-60; * is the point of attachment to the AA_{SC}, and AA_{SC} is a side chain of an amino acid residue of the cCPP; and M is a bonding group defined herein. The terminal NH of the linker may be conjugated to an EP via the terminal C of the EP.

The linker can have the structure:

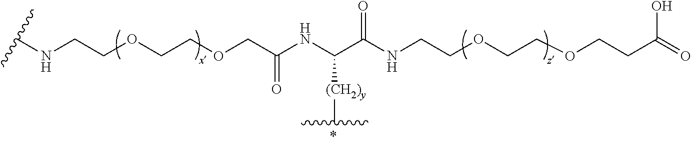

wherein: x′ is an integer from 1-60; y is an integer from 1-5; and z′ is an integer from 1-60; * is the point of attachment to the AA_{SC}, and AA_{SC} is a side chain of an amino acid residue of the cCPP. The terminal NH of the linker may be conjugated to an EP.

x′ can be an integer from 0-60, e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 29, 40, 41, 42, 43, 44, 45, 46, 47, 48, 49, 50, 51, 52, 53, 54, 55, 56, 57, 58, 59, or 60, inclusive of all ranges and subranges therebetween. x′ can Be an integer from 5-15. x′ can Be an integer from 9-13. x′ can be an integer from 1-5. x′ can be 1.

y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.

z can be an integer from 1-10, e.g., 1, 2, 3, 4, 5, 6, 7, 8, 9, or 10, inclusive of all ranges and subranges therebetween.

z′ can be an integer from 1-60, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 29, 40, 41, 42, 43, 44, 45, 46, 47, 48, 49, 50, 51, 52, 53, 54, 55, 56, 57, 58, 59, or 60, inclusive of all ranges and subranges therebetween. z′ can Be an integer from 5-15. z′ can Be an integer from 9-13. z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is part of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can have a structure:

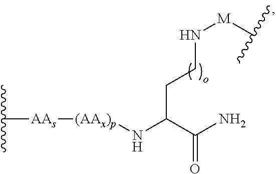

wherein:
M is a group that conjugates L to an AC;
AAₛ is a side chain or terminus of an amino acid on the cCPP;
each AAₓ is independently an amino acid residue;
o is an integer from 0 to 10; and
p is an integer from 0 to 5.

The linker can have a structure:

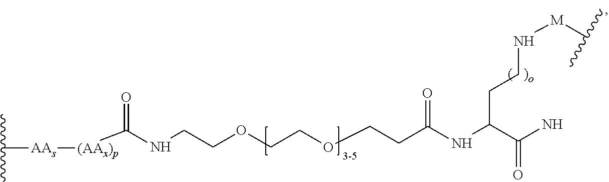

wherein:
M is a group that conjugates L to a cargo (e.g., an ASO);
AAₛ is a side chain or terminus of an amino acid on the cCPP;
each AAₓ is independently an amino acid residue;
o is an integer from 0 to 10; and
p is an integer from 0 to 5.

The linker can have a structure:

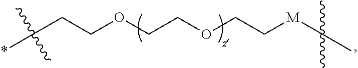

wherein:
M is a group that conjugates L to a cargo (e.g., an ASO);
* is the point of attachment of the linker to an AA_{SC} of a cCPP; and
z′ is an integer from 0 to 60.

M can comprise an alkylene, alkenylene, alkynylene, carbocyclyl, or heterocyclyl, each of which is optionally substituted. M can comprise or be selected from:

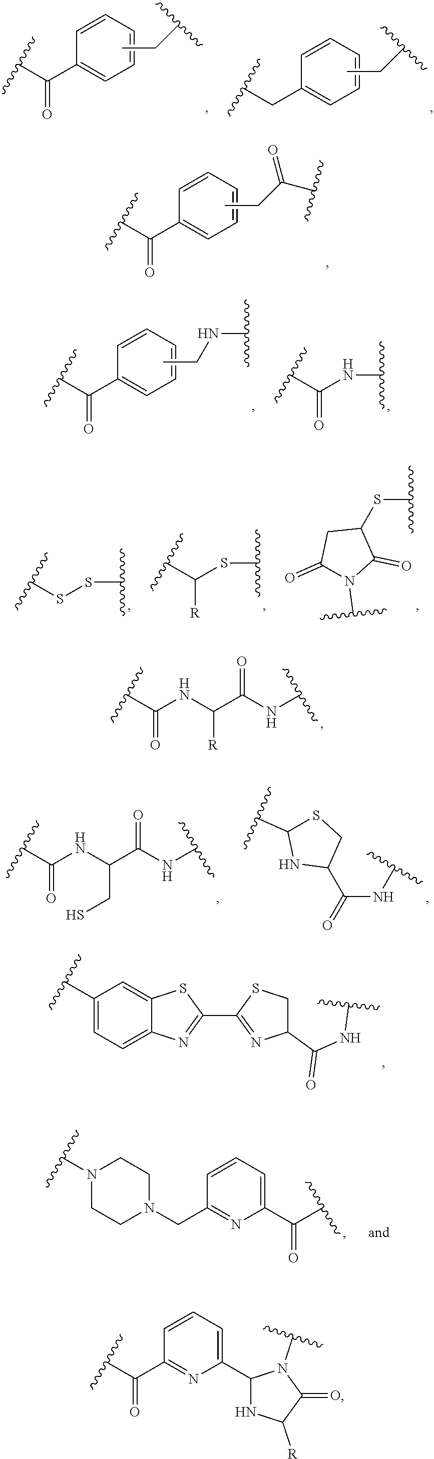

wherein R is alkyl, alkenyl, alkynyl, carbocyclyl, or heterocyclyl.

M can comprise or be selected from:

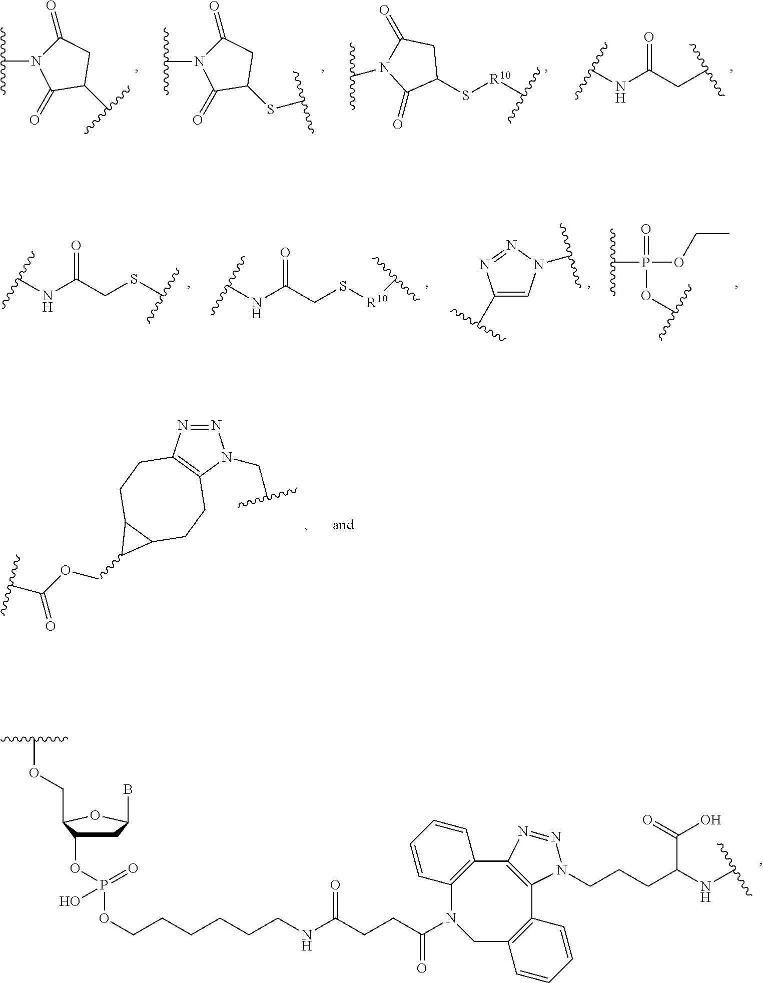

wherein: B is a nucleobase of an oligonucleotide cargo (e.g., the 3′ base of an oligonucleotide cargo); and R¹⁰ is alkylene, cycloalkyl, or

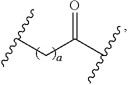

wherein a is 0 to 10.

M can comprise or be selected from:

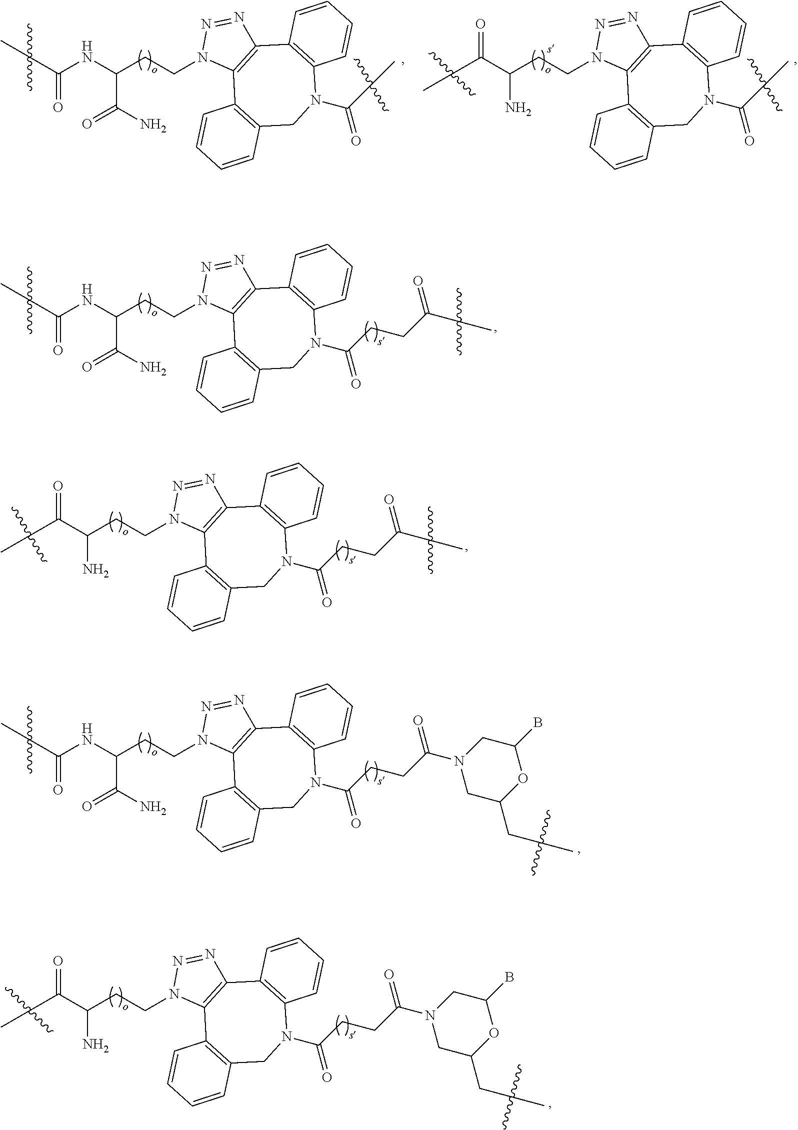

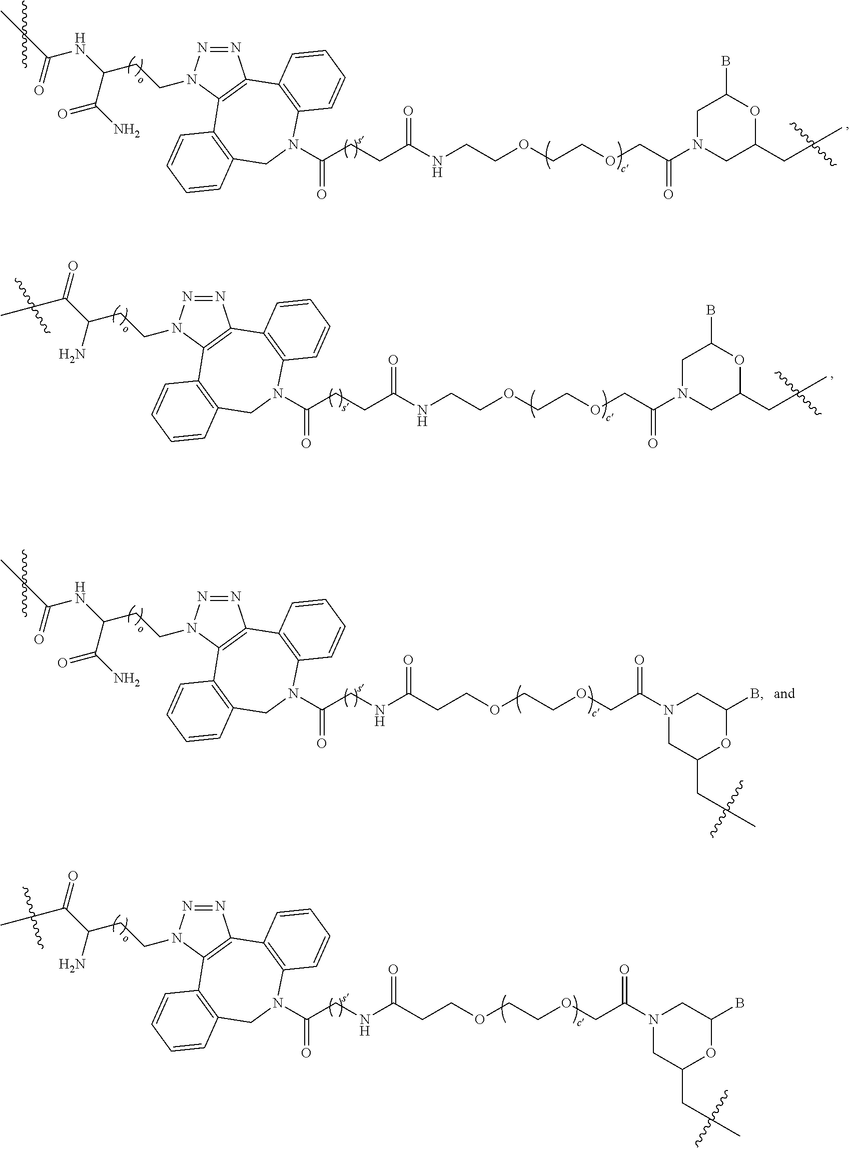

wherein o is an integer from 0 to 10 (e.g., 0, 1, 2, 3, 4, 5, 6, 7, 8, 9, or 10); s′ is an integer from 0 to 10 (e.g., 0, 1, 2, 3, 4, 5, 6, 7, 8, 9, or 10); c′ is an integer from 0 to 10 (e.g., 0, 1, 2, 3, 4, 5, 6, 7, 8, 9, or 10), and B is a nucleobase of an oligonucleotide cargo (e.g., the 3′ base of an oligonucleotide cargo).

M can be

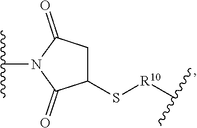

### R¹⁰ can be

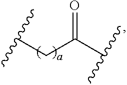

### and a is 0 to 10. M can be

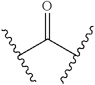 [SMILES: *C(*)=O]

M can be a heterobifunctional crosslinker, e.g.,

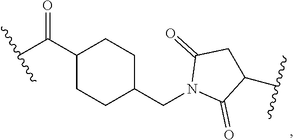 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

M can be or comprise

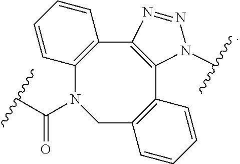 [SMILES: *C(=O)N1Cc2ccccc2-c2c(nnn2*)-c2ccccc21]

M can be or comprise

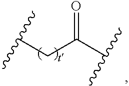

wherein t′ is 0 to 10. M can be

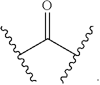 [SMILES: *C(*)=O]

M can be or comprise

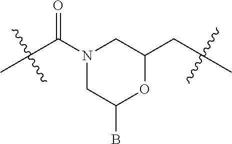

wherein B is a nucleobase of an oligonucleotide cargo (e.g., the 3′ base of an oligonucleotide cargo such as a PMO).

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The β-amino acid can be β-alanine.

o can be an integer from 0 to 10, e.g., 0, 1, 2, 3, 4, 5, 6, 7, 8, 9, and 10. o can be 0, 1, 2, or 3. o can be 0. o can be 1. o can be 2. o can be 3.

p can be 0 to 5, e.g., 0, 1, 2, 3, 4, or 5. p can be 0. p can be 1. p can be 2. p can be 3. p can be 4. p can be 5.

The linker can have the structure:

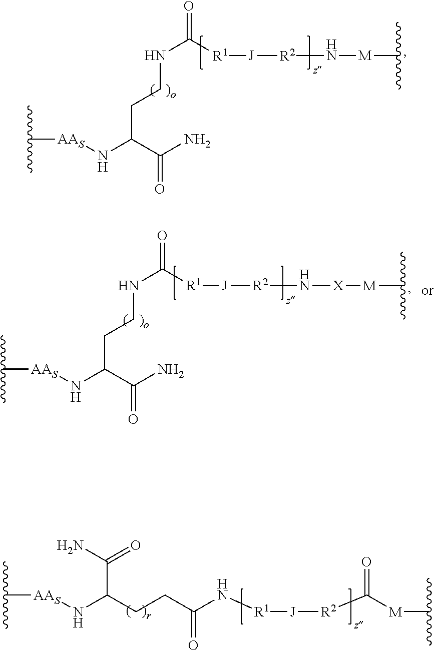

wherein M, AAₛ, each —(R¹ J-R²)z″-, o and z″ are defined herein; r can be 0 or 1.

In embodiments, r is 0. In embodiments, r is 1.

The linker can have the structure:

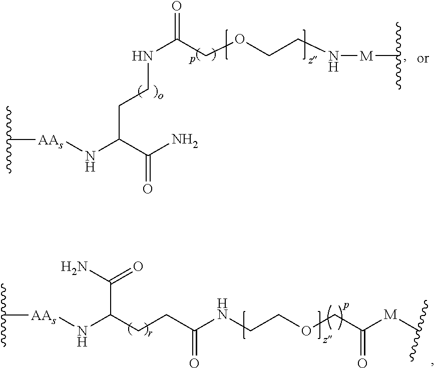

wherein each of M, AAₛ, o, p, q, r and z″ can be as defined herein.

z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges And values therebetween. z″ can Be an integer from 5-20. z″ can be an integer from 10-15.

The linker can have the structure:

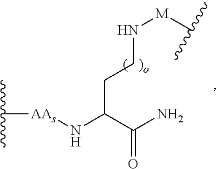

wherein:
M, AAₛ and o are as defined herein.

Other non-limiting examples of suitable linkers include:

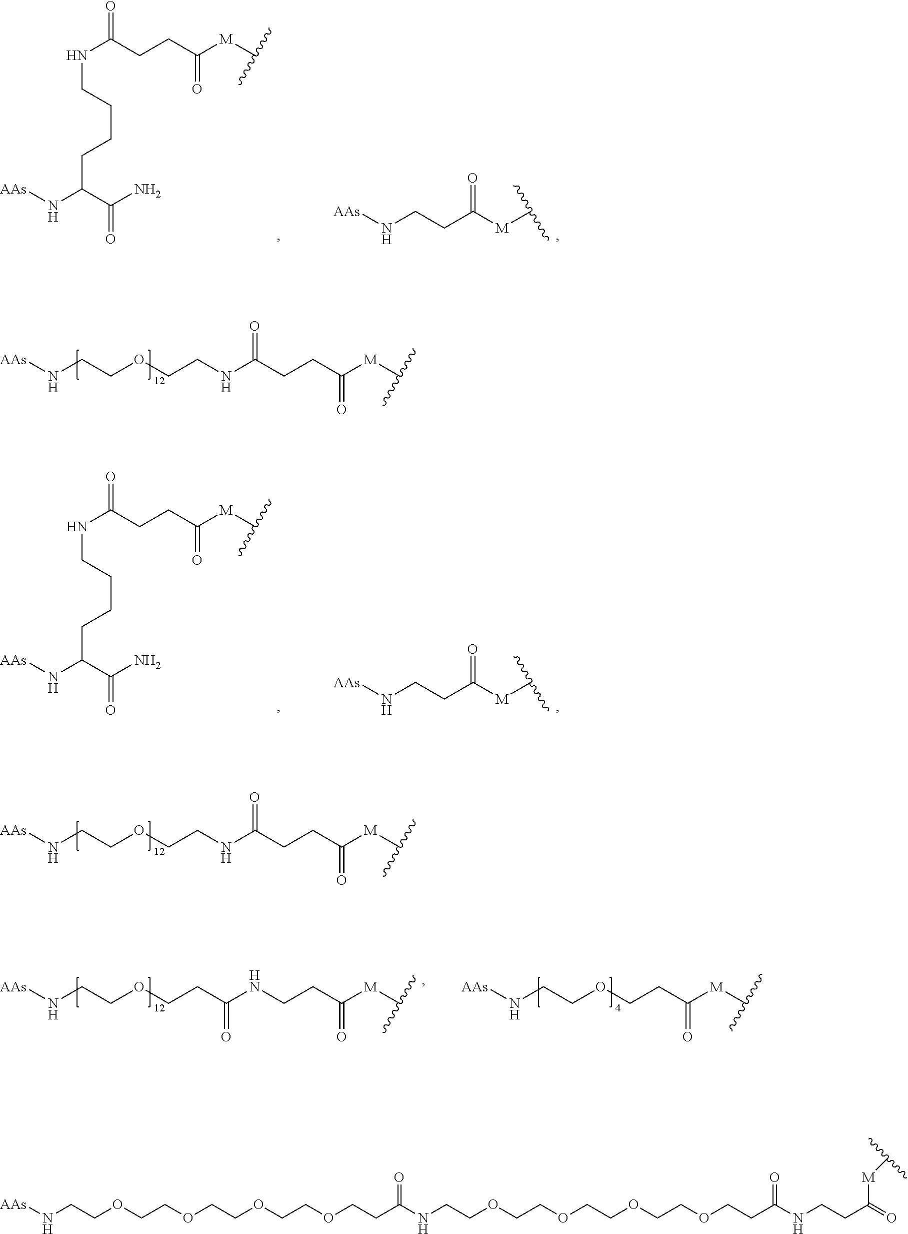

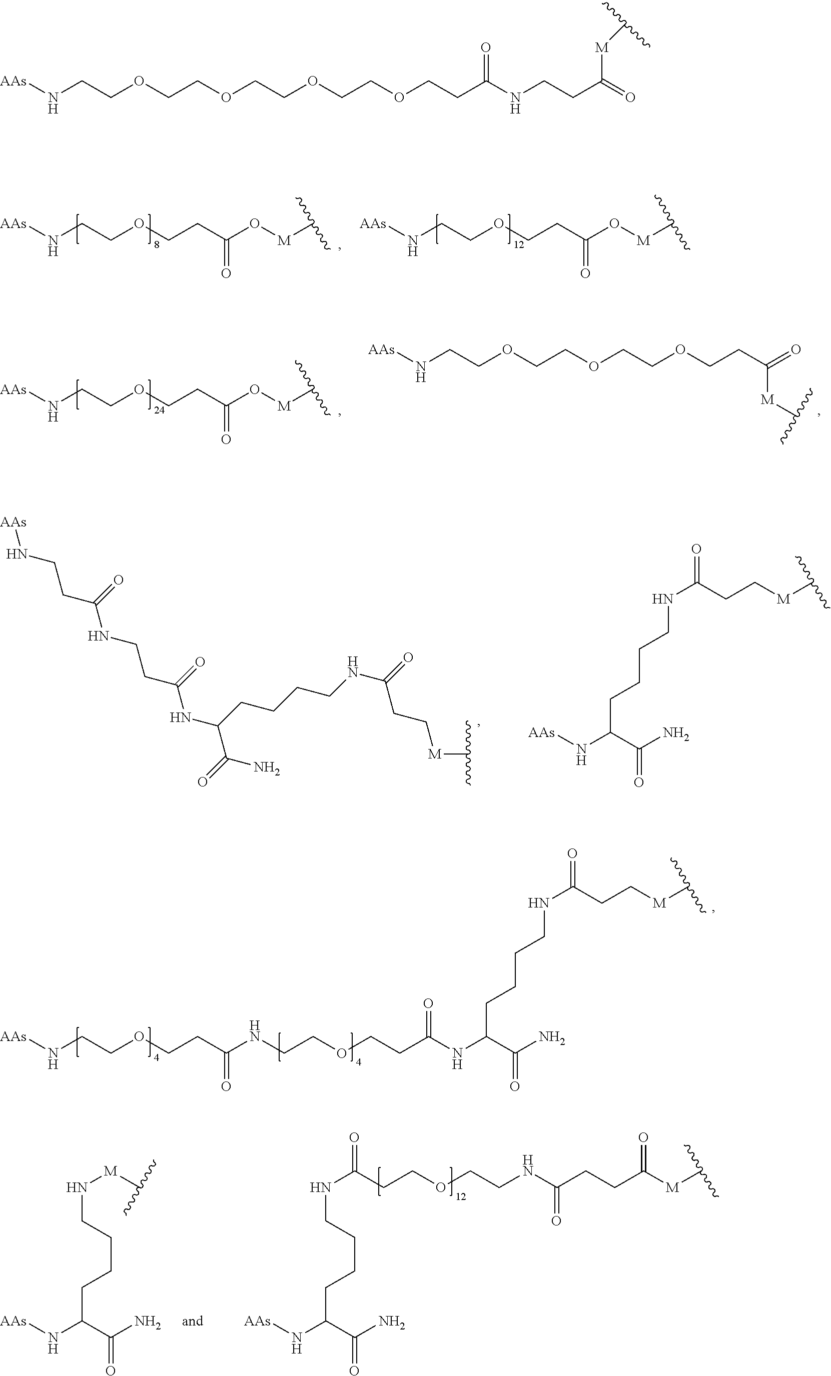

wherein M and AAₛ are as defined herein.

Provided herein is a compound comprising a cCPP and an AC that is complementary to a target in a pre-mRNA sequence further comprising L, wherein the linker is conjugated to the AC through a bonding group (M), wherein M is

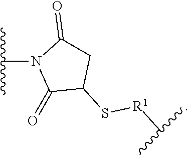

Provided herein is a compound comprising a cCPP and an antisense compound (AC), for example, an antisense oligonucleotide, that is complementary to a target in a pre-mRNA sequence, wherein the compound further comprises L, wherein the linker is conjugated to the AC through a bonding group (M), wherein M comprises or is selected from:

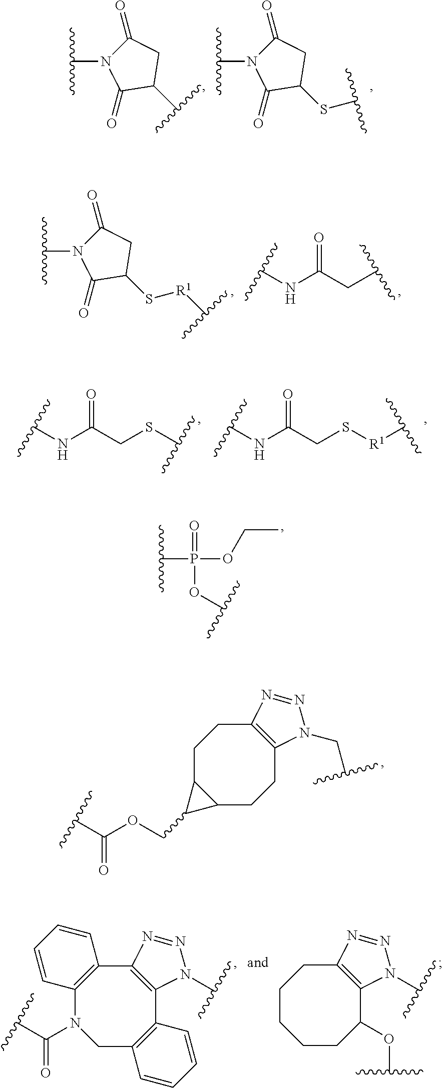

wherein: R¹ is alkylene, cycloalkyl, or

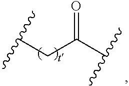

wherein t′ is 0 to 10 wherein each R is independently an alkyl, alkenyl, alkynyl, carbocyclyl, or heterocyclyl, wherein R¹ is

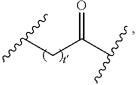

and t′ is 2.

M can be

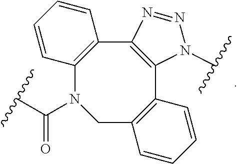 [SMILES: *C(=O)N1Cc2ccccc2-c2c(nnn2*)-c2ccccc21]

M can be or

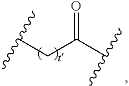

wherein t′ is 0 to 10. M can be

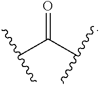 [SMILES: *C(*)=O]

The linker can have the structure:

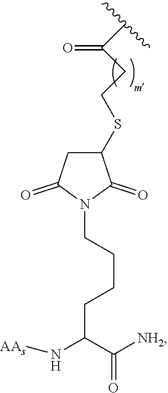

wherein AAₛ is as defined herein, and m′ is 0-10.

The linker can be of the formula:

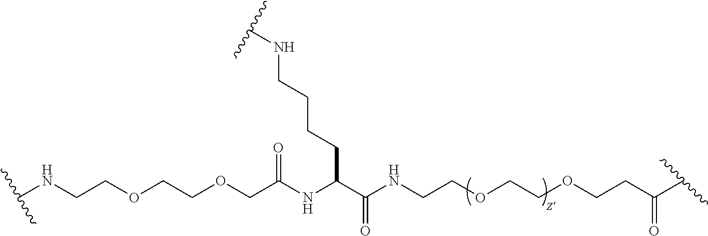

wherein z′ is defined herein.

The linker can be of the formula:

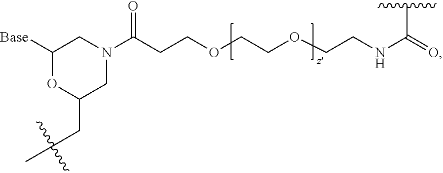

wherein “base” is a nucleobase of an oligonucleotide cargo (e.g., the 3′ base of an oligonucleotide cargo such as PMO); and z′ is defined herein.

The linker can be of the formula:

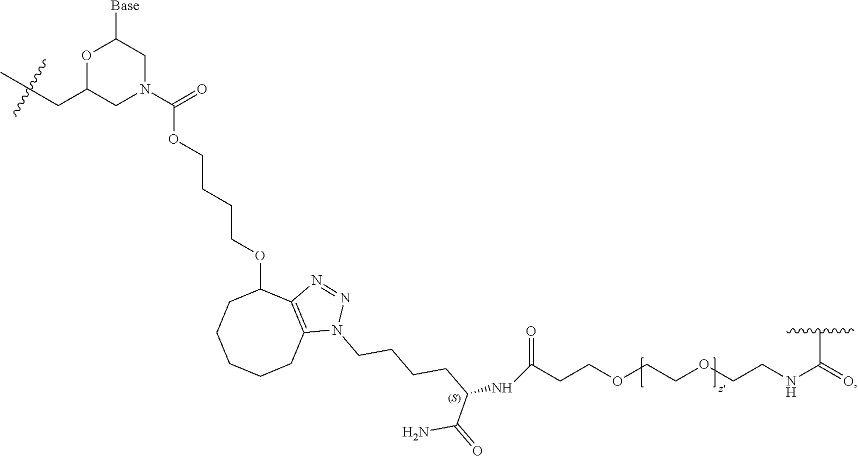

wherein “base” is a nucleobase of an oligonucleotide cargo (e.g., the 3′ base of an oligonucleotide cargo such as PMO); and z′ is defined herein. The linker can be of the formula:

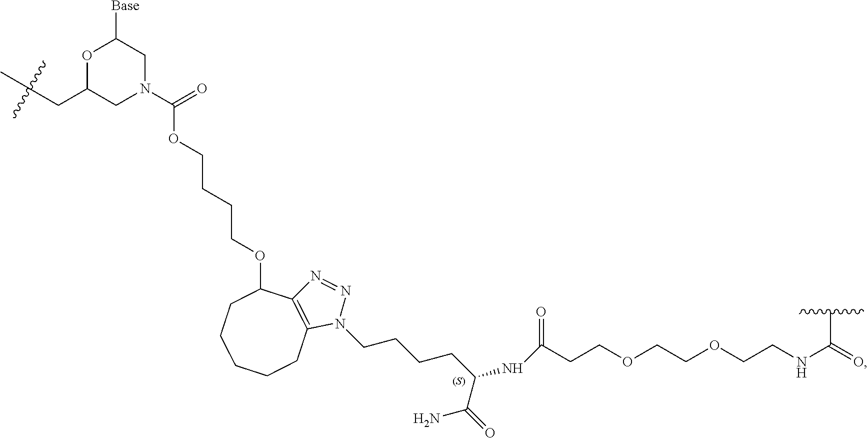

wherein “base” is a nucleobase of an oligonucleotide cargo (e.g., the 3′ base of an oligonucleotide cargo such as PMO).

The linker can be of the formula:

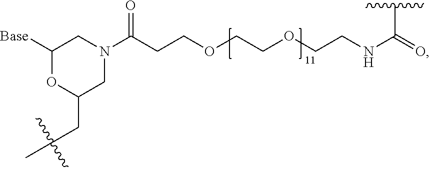

wherein “base” is a nucleobase of an oligonucleotide cargo (e.g., the 3′ base of an oligonucleotide cargo such as PMO).

The linker can be of the formula:

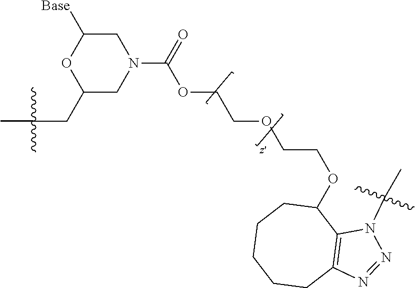

wherein: “base” is a nucleobase of an oligonucleotide cargo (e.g., the 3′ base of an oligonucleotide cargo such as a PMO), and z′ is defined herein.

The linker can comprise:

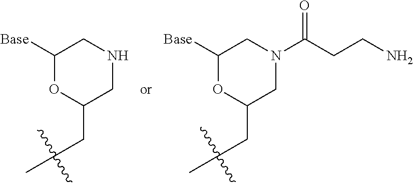

wherein: “base” a nucleobase of an oligonucleotide cargo (e.g., the 3′ base of an oligonucleotide cargo) base is a nucleobase of an oligonucleotide cargo.

The linker can comprise:

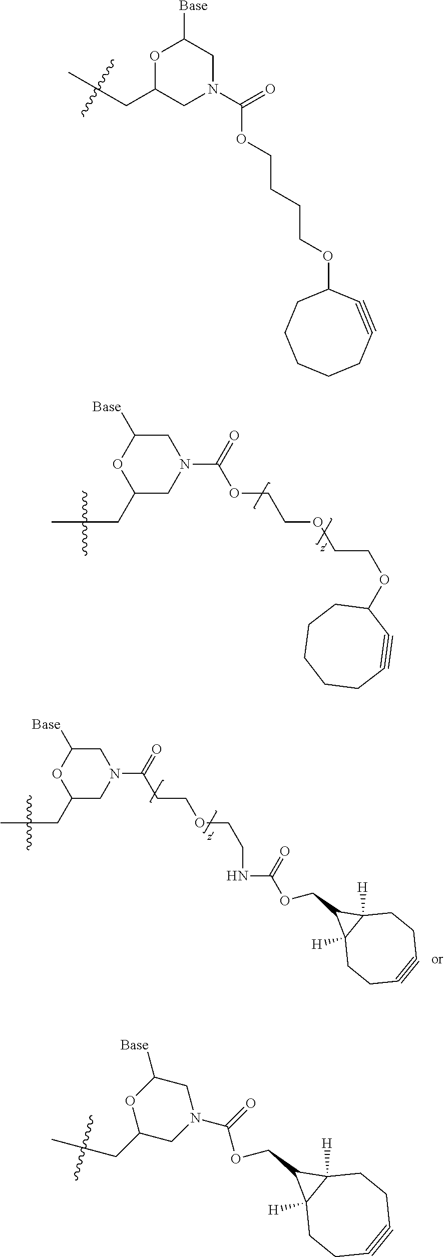

wherein: “base” is a nucleobase of an oligonucleotide cargo (e.g., the 3′ base of an oligonucleotide cargo); and z′ is defined herein.

The linker can be covalently bound to a cargo at any suitable location on the oligonucleotide cargo. The linker is covalently bound to the 3′ end of cargo or the 5′ end of an oligonucleotide cargo. The linker can be covalently bound to the backbone of an oligonucleotide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

### Delivery Constructs and Cargo Conjugates

The components of an ocular cargo conjugate can be conjugated to a linker defined herein. An ocular delivery construct can be linked to a cargo to from a cargo conjugate. The cargo can be linked to the ocular delivery construct through a linker. The cargo can be conjugated to the linker using any conjugation reaction disclosed herein to form a bonding group (M).

In embodiments, a cargo is directly conjugated to the cCPP of an ocular delivery construct to form a cargo conjugate. In embodiments, at least one atom of the cCPP can be replaced by a cargo or at least one lone pair can form a bond to a cargo. In embodiments, at least one atom of an amino acid side chain of the cCPP is replaced by a cargo or at least one lone pair of the atom forms a bond to a cargo. In embodiments, a hydrogen atom on the NH₂ group of the carboxamide side chain of a cCPP can be replaced by a bond to the cargo. In embodiments, a hydrogen atom on the NH₂ group of the carboxamide of a glutamine side chain of the cCPP can be replaced by a bond to the cargo.

In embodiments, a cargo is linked to an ocular delivery construct through a linker to form a cargo conjugate. In embodiments, the ocular delivery construct comprises a cCPP and a linker. In embodiments, the AA_{SC} of a cCPP is conjugated to a linker and the cargo is conjugated to the linker thereby forming a cargo conjugate.

The cargo can be any therapeutic moiety such as a peptide, oligonucleotide, or small molecule. The cargo can be a peptide sequence or a non-peptidyl therapeutic agent. The cargo can be a therapeutic moiety that is an antibody or an antigen binding fragment thereof, including, but not limited to an scFv or nanobody.

The cCPP can be conjugated to a linker defined herein. The linker can be conjugated to an AA_{SC} of the cCPP as defined herein.

The linker can comprise a —(OCH₂CH₂)_{z′}— subunit (e.g., as a spacer), wherein z′ is an integer from 1 to 60, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 29, 40, 41, 42, 43, 44, 45, 46, 47, 48, 49, 50, 51, 52, 53, 54, 55, 56, 57, 58, 59, or 60. “—(OCH₂CH₂)_{z′} is also referred to as PEG. In embodiments, z′ is 11. In embodiments, z′ is 1.

The linker can comprise a —(OCH₂CH₂)_{z′}— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z′} is also referred to as PEG. In embodiments, z′ is 11. In embodiments, z′ is 1.

An endosomal escape vehicle (EEV) can comprise a cyclic cell penetrating peptide (cCPP), an exocyclic peptide (EP) and linker, and can be conjugated to a cargo to form an EEV-conjugate (cargo conjugate) comprising the structure of Formula (Z):

EEVs comprising a cyclic cell penetrating peptide (cCPP), linker and exocyclic peptide (EP) are provided. A cargo construct comprising an EEV can comprise the structure of Formula (X):

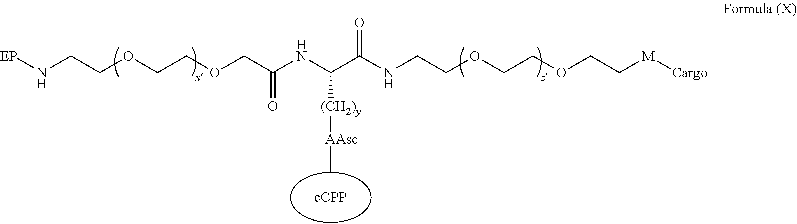

wherein: EP is an exocyclic peptide; cargo is a therapeutic moiety as defined herein; cCPP is a cCPP; and x′, y, z′, and M are defined herein. In embodiments, cCPP is a cCPP of Formula (I). In embodiments, cCPP is a cCPP of Formula (II).

A cargo construct comprising an EEV can comprise the structure of Formula (B):

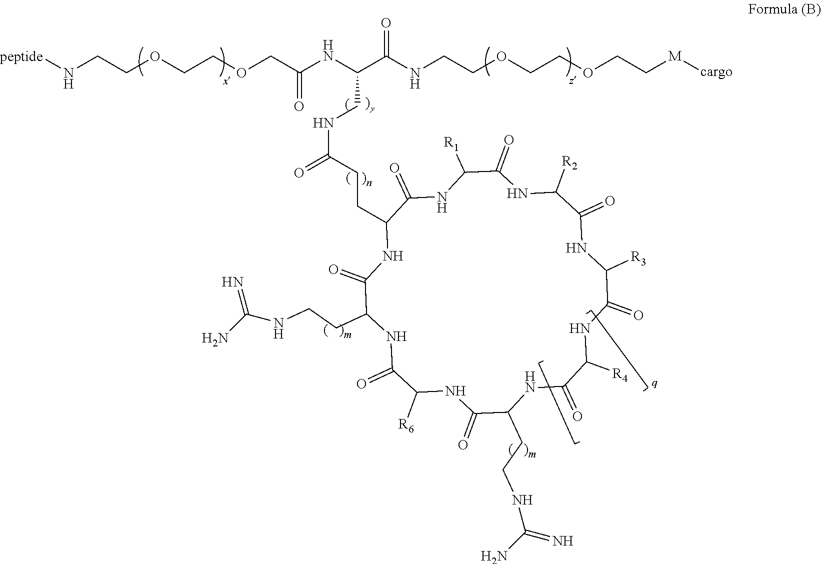

or a protonated form thereof,
wherein:
R₁, R₂, and R₃ are each independently H or an aryl or heteroaryl side chain of an amino acid;
R₄ and R₆ are independently H or an amino acid side chain;
peptide is an exocyclic peptide as defined herein;
M is a bonding group as defined herein;
each m is independently an integer from 0-3;
cargo is a therapeutic moiety as defined herein;
n is an integer from 0-2;
x′ is an integer from 1-20;
y is an integer from 1-5;
q is 1-4; and
z′ is an integer from 1-20.

In embodiments the cargo conjugate comprises the structure of Formula (B) or a protonated form thereof,
wherein:
R₁, R₂, and R₃ are each independently H or an aryl or heteroaryl side chain of an amino acid;
at least two of R₁, R₂, and R₃ are an aryl or heteroaryl side chain of an amino acid;
R₄ and R₆ are independently H or an amino acid side chain;
peptide is an exocyclic peptide (EP);
each m is independently an integer from 0-3;
cargo is a therapeutic moiety as defined herein;
n is an integer from 0-2;
x′ is an integer from 0-20;
y is an integer from 1-5;
q is 1-4; and
z′ is an integer from 0-20.

In embodiments the cargo conjugate comprises the structure of Formula (B) or a protonated form thereof wherein:
R₁, R₂, and R₃ can each independently be H or an amino acid residue having a side chain comprising an aryl group,
R₄ and R₆ are independently H or an amino acid side chain;
peptide is an exocyclic peptide as defined herein;
M is a bonding group as defined herein;
cargo is a therapeutic moiety as defined herein;
each m is independently an integer from 0-3;
n is an integer from 0-2;
x′ is an integer from 1-20;
y is an integer from 1-5;
q is an integer from 1-4; and
z′ is an integer from 1-20.

A cargo conjugate comprising an EEV can comprise the structure of Formula (B-1) or (B-2):

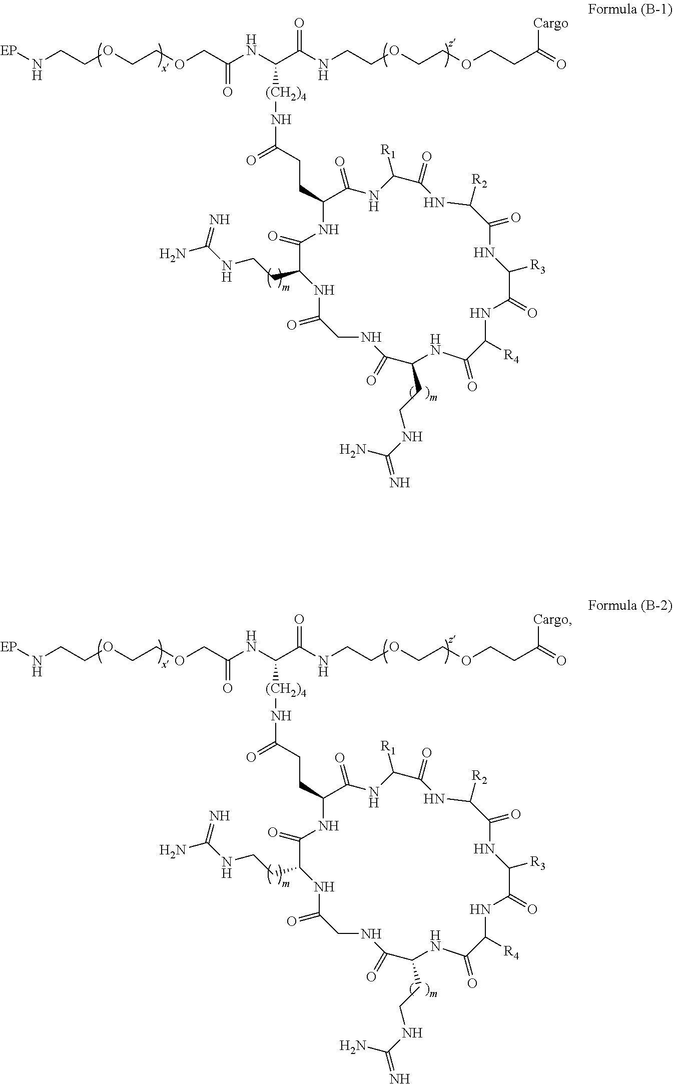

or a protonated form thereof,
wherein
   R₁, R₂, and R₅ are each independently H or an aryl or heteroaryl side chain of an amino acid;
R₄ is H or an amino acid side chain;
EP is an exocyclic peptide as defined herein;
each m is independently an integer from 0-3;
cargo is a therapeutic moiety as defined herein;
x′ is an integer from 1-20;
y is an integer from 1-5, and
z′ is an integer from 1-20.

A cargo conjugate comprising an EEV can comprise the structure of Formula (C):

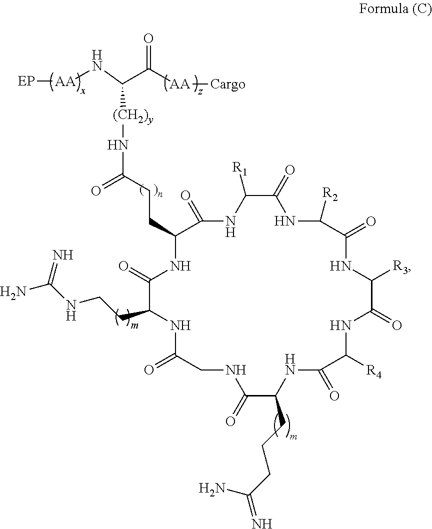

or a protonated form thereof, wherein EP, R¹, R², R³, R⁴, and m are as defined above in Formula (B); cargo is a therapeutic moiety as defined herein; AA can be an amino acid as defined herein; n can be an integer from 0-2; x can be an integer from 0-10; y can be an integer from 1-5; and z can be an integer from 1-10.

A cargo construct comprising an EEV can comprise the structure of Formula the structure of Formula (D):

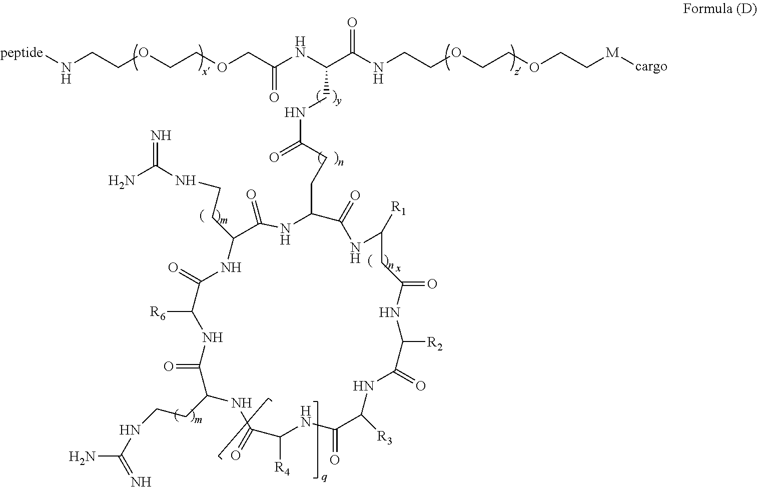

or a protonated form thereof,
wherein:
R₁, R₂, and R₃ can each independently be H or an amino acid residue having a side chain comprising an aryl or heteroaryl group;
at least two of R₁, R₂, and R₃ is an aryl or heteroaryl side chain of an amino acid;
R₄, and R₆ are independently H or an amino acid side chain;
AA_{SC} is an amino acid side chain;
nₓ is 0 or 1;
cargo is a therapeutic moiety as defined herein;
M is a bonding group as defined herein.
q is 1, 2, 3 or 4;
each m is independently an integer from 0-3;
n is an integer from 0-2;
x′ is an integer from 1-20;
y is an integer from 1-5;
q is an integer from 1-4; and
z′ is an integer from 1-20.

In embodiments, cCPP is a cCPP of Formula (X), (B), (B-1), (B-2), (C), or (D) wherein x′ is 1. In embodiments, cCPP is a cCPP of Formula (X), (B), (B-1), (B-2), (C), or (D) wherein y is 1. In embodiments, cCPP is a cCPP of Formula (X), (B), (B-1), (B-2), (C), or (D) wherein z′ is 2. In embodiments, cCPP is a cCPP of Formula (X), (B), (B-1), (B-2), (C), or (D) wherein z′ is 11. In embodiments, cCPP is a cCPP of Formula (X), (B), (B-1), (B-2), (C), or (D) wherein M comprises

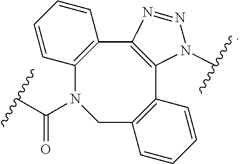 [SMILES: *C(=O)N1Cc2ccccc2-c2c(nnn2*)-c2ccccc21]

In embodiments, cCPP is a cCPP of Formula (B), (C), or (D) wherein M is or comprises

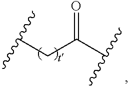

wherein t′ is 0 to 10. In embodiments, cCPP is a cCPP of Formula (B), (C), or (D) wherein M is or comprises

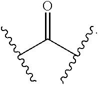 [SMILES: *C(*)=O]

### Delivery Constructs Before Conjugation to a Cargo

Delivery constructs having a structure prior to conjugation to a cargo are provided. The delivery constructs can include a cCPP, a cCPP and a linker, or an EEV (cCPP, EP, and linker). Prior to conjugation to the cargo, the linker can include a reactive group. The reactive group can react with a reactive group on a cargo to form at least a portion of a bonding group M as described herein. Examples of reactive groups include an azide (N₃) and a carboxylic acid. In embodiments, where the delivery construct prior to conjugation to a cargo includes an azide, click chemistry may be used to conjugate the delivery construct to a cargo. In embodiments, an azide reactive group may be incorporated in a delivery construct as an amino acid derivative, for example, azidolysine (K(N₃)). In embodiments, where the delivery construct prior to conjugation to a cargo includes a carboxylic acid, amid bond forming chemistry may be used to conjugate the delivery construct to a cargo.

A delivery construct prior to conjugation to a cargo can be selected from

cyclo[Ff-Nal-RrRrQ]-PEG_{z′}-OH;
and
cyclo[Ff-Nal-RrRrQ]-PEG_{z′}-K(N₃)—NH₂,

wherein x′ and z′ are, independently, an integer from 0 to 12.

A delivery construct prior to conjugation to a cargo can be selected from

Ac-PKKKRKV-PEG_{x′}-K(cyclo[GfFGrGrQ])-PEG_{z′}-OH;
and
Ac-PKKKRKV-PEG_{x′}-K(cyclo[GfFGrGrQ])-PEG_{z′}-
K(N₃)—NH₂

wherein x′ and z′ are, independently, an integer from 0 to 12.

A delivery construct prior to conjugation to a cargo can be selected from

Ac-PKKKRKV-PEG_{x′}-K(cyclo[FfFGRGRQ])-PEG_{z′}-OH;
and
Ac-PKKKRKV-PEGx′-K(cyclo[FfFGRGRQ])-PEG_{z′}-
K(N₃)—NH₂

wherein x′ and z′ are, independently, an integer from 0 to 12.

A delivery construct prior to conjugation to a cargo can be selected from

Ac-PKKKRKV-PEG_{x′}-K(cyclo[Ff-Nal-GrGrQ])-
PEG_{z′}-OH;
and
Ac-PKKKRKV-PEG_{x′}-K(cyclo[Ff-Nal-GrGrQ])-
PEG_{z′}-K(N₃)—NH₂

wherein x′ and z′ are, independently, an integer from 0 to 12.

A delivery construct prior to conjugation to a cargo can be selected from

Ac-PKKKRKV-PEG_{x′}-K(cyclo[FGFGRGRQ])-
PEG_{z′}-OH;
and
Ac-PKKKRKV-PEG_{x′}-K(cyclo[FGFGRGRQ])-
PEG_{z′}-K(N₃)—NH₂

wherein x′ and z′ are, independently, an integer from 0 to 12.

A delivery construct prior to conjugation to a cargo can be selected from

Ac-KKKRK-PEG_{x′}-K(cyclo(FGFGRGRQ))-
PEG_{z′}-OH;
and
Ac-KKKRK-PEG_{x′}-K(cyclo(FGFGRGRQ))-
PEG_{z′}-K(N₃)—NH₂

wherein x′ and z′ are, independently, an integer from 0 to 12.

A delivery construct prior to conjugation to a cargo can be selected from

Ac-PKKKRKV-PEG_{x′}-K(cyclo[FGFRRRRQ])-
PEG_{z′}-OH,
and
Ac-PKKKRKV-PEG_{x′}-K(cyclo[FGFRRRRQ])-
PEG_{z′}-K(N₃)—NH₂

wherein x′ and z′ are, independently, an integer from 0 to 12.

A delivery construct prior to conjugation to a cargo can be selected from

Ac-PKKKRKV-PEG_{x′}-K(cyclo[FFΦGRGRQ])-
PEG_{z′}-OH;
and
Ac-PKKKRKV-PEG_{x′}-K(cyclo[FFΦGRGRQ])-
PEG_{z′}-K(N₃)—NH₂

wherein x′ and z′ are, independently, an integer from 0 to 12.

A delivery construct prior to conjugation to a cargo can be selected from

Ac-PKKKRKV-PEG_{x′}-K(cyclo[βhF-FΦSRSRQ])-PEG_{z′}-
OH;
and
Ac-PKKKRKV-PEG_{x′}-K(cyclo[βhF-FΦSRSRQ])-PEG_{z′}-K
(N₃)—NH₂

wherein x′ and z′ are, independently, an integer from 0 to 12.

A delivery construct prior to conjugation to a cargo can be selected from

Ac-PKKKRKV-PEG_{x′}-K(cyclo[βhF-FΦGRGRQ])-PEG_{z′}-OH;
and
Ac-PKKKRKV-PEG_{x′}-K(cyclo[βhF-FΦGRGRQ])-PEG_{z′}-K(N₃)—
NH₂,

wherein x′ and z′ are, independently, an integer from 0 to 12.

A delivery construct prior to conjugation to a cargo can be selected from

Ac-PKKKRKV-PEG_{x′}-K(cyclo[bhF-f-ΦGrGrQ])-PEG_{z′}-OH;
and
Ac-PKKKRKV-PEG_{x′}-K(cyclo[bhF-f-ΦGrGrQ])-PEG_{z′}-K(N₃)—
NH₂,

wherein x′ and z′ are, independently, an integer from 0 to 12.

A delivery construct prior to conjugation to a cargo can be selected from

Ac-PKKKRKV-PEG_{x′}-K(cyclo[bhF-f-ΦGrGrQ])-PEG_{z′}-OH,
and
Ac-PKKKRKV-PEG_{x′}-K(cyclo[bhF-fΦSRSRQ])-PEG_{z′}-K(N₃)—
NH₂,

wherein x′ and z′ are, independently, an integer from 0 to 12.

K(N₃) is azidolysine; and a terminal OH indicates a terminal carboxylic acid. In embodiments, PEG₂ is miniPEG₂ or miniPEG. In embodiments, x′ is 0. In embodiments, x′ is 2. In embodiments, x′ is 4. In embodiments, x′ is 8. In embodiments, x′ is 12. In embodiments, z′ is 0. In embodiments, z′ is 2. In embodiments, z′ is 4. In embodiments, z′ is 8. In embodiments, z′ is 12.

A delivery construct prior to conjugation to a cargo can be selected from

cyclo[Ff-Nal-RrRrQ]-PEG₁₂-OH;
cyclo[Ff-Nal-RrRrQ]-PEG₁₂-K(N₃)—NH₂;
Ac-PKKKRKV-K(cyclo[Ff-Nal-GrGrQ])-PEG₁₂-K(N₃)—NH₂;
Ac-PKKKRKV-K(cyclo[Ff-Nal-GrGrQ])-miniPEG₂-K(N₃)—
NH₂;
Ac-PKKKRKV-PEG₂-K(cyclo[Ff-Nal-GrGrQ])-PEG₁₂-OH;
and
Ac-PKKKRKV-miniPEG₂-K(cyclo[FFΦGRGRQ])-PEG₂-K(N₃)—
NH₂.

A delivery construct prior to conjugation to a cargo can be selected from

Ac-PKKKRKV-PEG₂-K(cyclo[GfFGrGrQ])-PEG₂-K(N₃)—NH₂;
Ac-PKKKRKV-PEG₂-K(cyclo[FfFGRGRQ])-PEG₂-K(N₃)—NH₂;
Ac-PKKKRKV-PEG₂-K(cyclo[FGFGRGRQ])-PEG₁₂-OH;
Ac-PKKKRKV-PEG₂-K(cyclo[FGFRRRRQ])-PEG₁₂-OH;
Ac-PKKKRKV-miniPEG₂-K(cyclo[FGFGRGRQ])-PEG₁₂-K(N₃)—
NH₂;
Ac-PKKKRKV-miniPEG₂-K(cyclo[FGFGRGRQ])-miniPEG₂-K
(N₃)—NH₂;
and
Ac-PKKKRKV-miniPBG₂-K(cyclo[FfFGRGRQ])-miniPEG₂-K
(N₃)—NH₂.

A delivery construct prior to conjugation to a cargo can be:

Ac-KKKRK-miniPEG₂-K(cyclo(FGFGRGRQ))-miniPEG₂-K
(N₃)—NH₂.

A delivery construct prior to conjugation to a cargo can be selected from

Ac-PKKKRKV-miniPEG₂-K(cyclo[βhF-FΦSRSRQ])-
PEG₁₂-OH;
and
Ac-PKKKRKV-miniPEG₂-K(cyclo[βhF-FΦGRGRQ])-
PEG₁₂-OH.
Ac-PKKKRKV-miniPEG₂-K(cyclo[bhF-f-ΦGrGrQ])-
PEG₁₂-OH;
and
Ac-PKKKRKV-miniPEG₂-K(cyclo[bhF-fΦSRSRQ])-
PEG₁₂-OH.

It is understood that a terminal OH indicates a terminal carboxylic acid and K(N₃) is azidolysine. In embodiments, PEG₂ is miniPEG₂ or miniPEG.

In embodiments, a delivery construct having an azide group that can be used in a click chemistry reaction to conjugate a cargo to the delivery construct can be selected from

cyclo[Ff-Nal-RrRrQ]-PEG_{z′}-K(N₃)—NH₂
Ac-PKKKRKV-PEG_{x′}-K(cyclo[GfFGrGrQ])-PEG_{z′}-K(N₃)—NH₂;
Ac-PKKKRKV-PEG_{x′}-K(cyclo[FfFGRGRQ])-PEG_{z′}-K(N₃)—NH₂;
Ac-PKKKRKV-PEG_{x′}-K(cyclo[Ff-Nal-GrGrQ])-PEG_{z′}-K
(N₃)—NH₂;
Ac-PKKKRKV-PEG_{x′}-K(cyclo[FGFGRGRQ])-PEG_{z′}-K(N₃)—NH₂;
Ac-PKKKRKV-PEG_{x′}-K(cyclo[FGFRRRRQ])-PEG_{z′}-K(N₃)—NH₂;
Ac-PKKKRKV-K(cyclo[Ff-Nal-G-r-G-rQ])-PEG_{z′}-K(N₃)—
NH₂;
Ac-PKKKRKV-K(cyclo[Ff-Nal-G-r-G-rQ])-PEG_{z′}-K(N₃)—
NH₂;
Ac-PKKKRKV-PEG_{x′}-K(cyclo[FGFGRGRQ])-PEG_{z′}-K(N₃)—NH₂;
Ac-PKKKRKV-PEG_{x′}-K(cyclo[FfFGRGRQ])-PEG_{z′}-K(N₃)—NH₂;
Ac-KKKRK-PEG_{x′}-K(cyclo[FGFGRGRQ])-PEG_{z′}-K(N₃)—NH₂;
Ac-PKKKRKV-PEG_{x′}-K(cyclo[FFΦGRGRQ])-PEG_{z′}-K(N₃)—NH₂;
Ac-PKKKRKV-PEG_{x′}-K(cyclo[βhF-FΦSRSRQ])-PEG_{z′}-K(N₃)—
NH₂;
Ac-PKKKRKV-PEG_{x′}-K(cyclo[βhF-FΦGRGRQ])-PEG_{z′}-K(N₃)—
NH₂;
and
Ac-PKKKRKV-PEG_{x′}-K(cyclo[FGFGRGRQD)-PEG_{z′}-K(N₃)—NH₂;

wherein K(N₃) is azidolysine; and x′ and z′ are, independently, an integer from 0 to 12. In embodiments, x′ is 0. In embodiments, x′ is 2. In embodiments, x′ is 4. In embodiments, x′ is 8. In embodiments, x′ is 12. In embodiments, z′ is 0. In embodiments, z′ is 2. In embodiments, z′ is 4. In embodiments, z′ is 8. In embodiments, z′ is 12. In embodiments, PEG_{x′} is miniPEG₂. In embodiments, PEG_{z′} is miniPEG₂ or miniPEG.

In embodiments, a delivery construct having an azide group that can be used in a click chemistry reaction to conjugate a cargo to the delivery construct can be cyclo[Ff-Nal-RrRrQ]-PEG₂-K(N₃)—NH₂. In embodiments, the delivery construct can be cyclo[Ff-Nal-RrRrQ]-PEG₁₂-K(N₃)—NH₂.

In embodiments, a delivery construct having an azide group that can be used in a click chemistry reaction to conjugate a cargo to the delivery construct can be Ac-PKKKRKV-PEG₂-K(cyclo[GfFGrGrQ])-PEG₂-K(N₃)—NH₂ where each PEG₂ may, independently, be miniPEG₂. In embodiments, the delivery construct can be Ac-PKKKRKV-PEG₂-K(cyclo[GfFGrGrQ])-PEG₁₂-K(N₃)—NH₂ where PEG₂ may be miniPEG₂.

In embodiments, a delivery construct having an azide group that can be used in a click chemistry reaction to conjugate a cargo to the delivery construct can be Ac-PKKKRKV-PEG₂-K(cyclo[FfFGRGRQ])-PEG₂-K(N₃)—NH₂ where each PEG₂ may, independently, be miniPEG₂. In embodiments, the delivery construct can be Ac-PKKKRKV-PEG₂-K(cyclo[FfFGRGRQ])-PEG₁₂-K(N₃)—NH₂ where PEG₂ may be miniPEG₂.

In embodiments, the delivery construct can be Ac-PKKKRKV-PEG₂-K(cyclo[Ff-Nal-GrGrQ])-PEG₂-K(N₃)—NH₂ where each PEG₂ may, independently, be miniPEG₂. In embodiments, the delivery construct can be Ac-PKKKRKV-PEG₂-K(cyclo[Ff-Nal-GrGrQ])-PEG₁₂-K(N₃)—NH₂ where PEG₂ may be miniPEG₂.

In embodiments, the delivery construct can be Ac-PKKKRKV-PEG₂-K(cyclo[FGFGRGRQ])-PEG₂ K(N₃)—NH₂ where each PEG₂ may, independently, be miniPEG₂. In embodiments, the delivery construct can be Ac-PKKKRKV-PEG₂-K(cyclo[FGFGRGRQ])-PEG₁₂ K(N₃)—NH₂ where PEG₂ may be miniPEG₂.

In embodiments, the delivery construct can be Ac-PKKKRKV-PEG₂-K(cyclo[FGFRRRRQ])-PEG: K(N₃)—NH₂ where each PEG₂ may, independently, be miniPEG₂. In embodiments, the delivery construct can be Ac-PKKKRKV-PEG₂-K(cyclo[FGFRRRRQ])-PEG₁₂ K(N₃)—NH₂ where PEG₂ may be miniPEG₂.

In embodiments, the delivery construct can be Ac-PKKKRKV-K(cyclo[Ff-Nal-G-r-G-rQ])-PEG₂ K(N₃)—NH₂ where each PEG₂ may, independently, be miniPEG₂. In embodiments, the delivery construct can be Ac-PKKKRKV-K(cyclo[Ff-Nal-G-r-G-rQ])-PEG₁₂ K(N₃)—NH₂ where PEG₂ may be miniPEG₂.

In embodiments, the delivery construct can be Ac-PKKKRKV-K(cyclo[Ff-Nal-G-r-G-rQ])-PEG₂ K(N₃)—NH₂ where each PEG₂ may, independently, be miniPEG₂. In embodiments, the delivery construct can be Ac-PKKKRKV-K(cyclo[f-Nal-G-r-G-rQ])-PEG₁₂ K(N₃)—NH₂ where PEG₂ may be miniPEG₂.

In embodiments, the delivery construct can be Ac-PKKKRKV-PEG₂-K(cyclo[FGFGRGRQ])-PEG₂-K(N₃)—NH₂ where each PEG₂ may, independently, be miniPEG₂. In embodiments, the delivery construct can be Ac-PKKKRKV-PEG₂-K(cyclo[FGFGRGRQ])-PEG₁₂-K(N₃)—NH₂ where PEG₂ may be miniPEG₂.

In embodiments, the delivery construct can be Ac-PKKKRKV-PEG₂-K(cyclo[FfFGRGRQ])-PEG₂-K(N₃)—NH₂ where each PEG₂ may, independently, be miniPEG₂. In embodiments, the delivery construct can be Ac-PKKKRKV-PEG₂-K(cyclo[FfFGRGRQ])-PEG₁₂-K(N₃)—NH₂ where PEG₂ may be miniPEG₂.

In embodiments, the delivery construct can be Ac—KKKRK-PEG₂-K(cyclo (FGFGRGRQ)-PEG₂-K(N₃)—NH₂ where each PEG₂ may, independently, be miniPEG₂. In embodiments, the delivery construct can be Ac—KKKRK-PEG₂-K(cyclo (FGFGRGRQ))-PEG₁₂-K(N₃)—NH₂ where PEG₂ may be miniPEG₂.

In embodiments, the delivery construct can be Ac-PKKKRKV-PEG₂-K(cyclo[FFΦGRGRQ])-PEG₂-K(N₃)—NH, where each PEG₂ may, independently, be miniPEG₂. In embodiments, the delivery construct can be Ac-PKKKRKV-PEG₂-K(cyclo[FFΦGRGRQ])-PEG₁₂-K(N₃)—NH₂ where PEG₂ may be miniPEG₂.

In embodiments, the delivery construct can be Ac-PKKKRKV-PEG₂-K(cyclo[βhF-FΦSRSRQ])-PEG₂-K(N₃)—NH₂ where each PEG₂ may, independently, be miniPEG₂. In embodiments, the delivery construct can be Ac-PKKKRKV-PEG₂-K(cyclo[βhF-FΦSRSRQ])-PEG₁₂-K(N₃)—NH₂ where PEG₂ may be miniPEG₂.

In embodiments, the delivery construct can be Ac-PKKKRKV-PEG₂-K(cyclo[βhF-FΦGRGRQD])-PEG₂-K(N₃)—NH₂ where each PEG₂ may, independently, be miniPEG₂. In embodiments, the delivery construct can be Ac-PKKKRKV-PEG₂-K(cyclo[βhF-FΦGRGRQ])-PEG₁-K(N₃)—NH₂ where PEG₂ may be miniPEG₂.

In embodiments, the delivery construct can be Ac-PKKKRKV-PEG₂-K(cyclo[FGFGRGRQ])-PEG₂-K(N₃)—NH₂ where each PEG₂ may, independently, be miniPEG₂. In embodiments, the delivery construct can be Ac-PKKKRKV-PEG₂-K(cyclo[FGFGRGRQ])-PEG₁₂-K(N₃)—NH₂ where PEG₂ may be miniPEG₂.

In embodiments, a delivery construct having an azide group that can be used in a click chemistry reaction to conjugate a cargo to the delivery construct can be selected from

Ac-PKKKRKV-PEG_{x′}-K(cyclo[GfFGrGrQ])-PEG_{z′}-
OH;
Ac-PKKKRKV-PEG_{x′}-K(cyclo[FfFGRGRQ])-PEG_{z′}-
OH;
Ac-PKKKRKV-PEG_{x′}-K(cyclo[Ff-Nal-GrGrQ])-
PEG_{z′}-OH;
Ac-PKKKRKV-PEG_{x′}-K(cyclo[FGFGRGRQ])-PEG_{z′}-
OH;
Ac-PKKKRKV-PEG_{x′}-K(cyclo[FGFRRRRQ])-PEG_{z′}-
OH;
Ac-PKKKRKV-K(cyclo[Ff-Nal-G-r-G-rQ])-
PEG_{z′}-OH;
Ac-PKKKRKV-K(cyclo[Ff-Nal-G-r-G-rQ])-
PEG_{z′}-OH;
Ac-PKKKRKV-PEG_{x′}-K(cyclo[FGFGRGRQ])-PEG_{z′}-
OH;
Ac-PKKKRKV-PEG_{x′}-K(cyclo[FfFGRGRQ])-PEG_{z′}-
OH;
Ac-KKKRK-PEG_{x′}-K(cyclo[FGFGRGRQ])-PEG_{z′}-OH;
Ac-PKKKRKV-PEG_{x′}-K(cyclo[FFΦGRGRQ])-PEG_{z′}-
OH;
Ac-PKKKRKV-PEG_{x′}-K(cyclo[βhF-FΦSRSRQ])-
PEG_{z′}-OH;
Ac-PKKKRKV-PEG_{x′}-K(cyclo[βhF-FΦGRGRQ])-
PEG_{z′}-OH;
and
Ac-PKKKRKV-PEG_{x′}-K(cyclo[FGFGRGRQ])-PEG_{z′}-
OH;

wherein OH indicates a terminal carboxylic acid; and x′ and z′ are, independently, an integer from 0 to 12. In embodiments, x′ is 0. In embodiments, x′ is 2. In embodiments, x′ is 4. In embodiments, x′ is 8. In embodiments, x′ is 12. In embodiments, z′ is 0. In embodiments, z′ is 2. In embodiments, z′ is 4. In embodiments, z′ is 8. In embodiments, z′ is 12. In embodiments, PEG, is miniPEG₂. In embodiments, PEG_{z′} is miniPEG₂.

In embodiments, a delivery construct having an azide group that can be used in a click chemistry reaction to conjugate a cargo to the delivery construct can be cyclo[Ff-Nal-RrRrQ]-PEG₂-OH. In embodiments, the delivery construct can be cyclo[Ff-Nal-RrRrQ]-PEG₁₂-OH.

In embodiments, a delivery construct having an azide group that can be used in a click chemistry reaction to conjugate a cargo to the delivery construct can be Ac-PKKKRKV-PEG₂-K(cyclo[GfFGrGrQ])-PEG₂-OH where each PEG₂ may, independently, be miniPEG₂. In embodiments, the delivery construct can be Ac-PKKKRKV-PEG₂-K(cyclo[GfFGrGrQD])-PEG₁₂-OH where PEG₂ may be miniPEG₂.

In embodiments, a delivery construct having an azide group that can be used in a click chemistry reaction to conjugate a cargo to the delivery construct can be Ac-PKKKRKV-PEG₂-K(cyclo[FfFGRGRQ])-PEG₂-OH where each PEG₂ may, independently, be miniPEG₂. In embodiments, the delivery construct can be Ac-PKKKRKV-PEG₂-K(cyclo[FfFGRGRQ])-PEG₁₂-OH where PEG₂ may be miniPEG₂.

In embodiments, the delivery construct can be Ac-PKKKRKV-PEG₂-K(cyclo[Ff-Nal-GrGrQ])-PEG₂-OH where each PEG₂ may, independently, be miniPEG₂. In embodiments, the delivery construct can be Ac-PKKKRKV-PEG₂-K(cyclo[Ff-Nal-GrGrQ])-PEG₁₂-OH where PEG₂ may be miniPEG₂.

In embodiments, the delivery construct can be Ac-PKKKRKV-PEG₂-K(cyclo[FGFGRGRQ])-PEG₂-OH where each PEG₂ may, independently, be miniPEG₂. In embodiments, the delivery construct can be Ac-PKKKRKV-PEG₂-K(cyclo[FGFGRGRQ])-PEG₁₂-OH where PEG₂ may be miniPEG₂.

In embodiments, the delivery construct can be Ac-PKKKRKV-PEG₂-K(cyclo[FGFRRRRQ])-PEG₂-OH where each PEG₂ may, independently, be miniPEG₂. In embodiments, the delivery construct can be Ac-PKKKRKV-PEG₂-K(cyclo[FGFRRRRQ])-PEG₁₂-OH where PEG₂ may be miniPEG₂.

In embodiments, the delivery construct can be Ac-PKKKRKV-K(cyclo[Ff-Nal-G-r-G-rQ])-PEG₂-OH where each PEG₂ may, independently, be miniPEG₂. In embodiments, the delivery construct can be Ac-PKKKRKV-K(cyclo[Ff-Nal-G-r-G-rQ])-PEG₁₂-OH where PEG₂ may be miniPEG₂.

In embodiments, the delivery construct can be Ac-PKKKRKV-K(cyclo[Ff-Nal-G-r-G-rQ])-PEG₂ K(N₃)—NH₂ where each PEG₂ may, independently, be miniPEG₂. In embodiments, the delivery construct can be Ac-PKKKRKV-K(cyclo[Ff-Nal-G-r-G-rQ])-PEG₁₂-OH where PEG₂ may be miniPEG₂.

In embodiments, the delivery construct can be Ac-PKKKRKV-PEG₂-K(cyclo[FGFGRGRQ])-PEG₂-OH where each PEG₂ may, independently, be miniPEG₂. In embodiments, the delivery construct can be Ac-PKKKRKV-PEG₂-K(cyclo[FGFGRGRQ])-PEG₁₂-OH where PEG₂ may be miniPEG₂.

In embodiments, the delivery construct can be Ac-PKKKRKV-PEG₂-K(cyclo[FfFGRGRQ])-PEG₂-OH where each PEG₂ may, independently, be miniPEG₂. In embodiments, the delivery construct can be Ac-PKKKRKV-PEG₂-K(cyclo[FfFGRGRQ])-PEG₁₂-OH where PEG₂ may be miniPEG₂.

In embodiments, the delivery construct can be Ac—KKKRK-PEG₂-K(cyclo (FGFGRGRQ)-PEG₂-OH where each PEG₂ may, independently, be miniPEG₂. In embodiments, the delivery construct can be Ac—KKKRK-PEG₂-K(cyclo[FGFGRGRQ])-PEG₁₂-OH where PEG₂ may be miniPEG₂.

In embodiments, the delivery construct can be Ac-PKKKRKV-PEG₂-K(cyclo[FFΦGRGRQ])-PEG₂-OH where each PEG₂ may, independently, be miniPEG₂. In embodiments, the delivery construct can be Ac-PKKKRKV-PEG₁₂-K(cyclo[FFΦGRGRQ])-PEG₁-OH where PEG₂ may be miniPEG₂.

In embodiments, the delivery construct can be Ac-PKKKRKV-PEG₂-K(cyclo[βhF-FΦSRSRQ])-PEG₂-OH where each PEG₂ may, independently, be miniPEG₂. In embodiments, the delivery construct can be Ac-PKKKRKV-PEG₂-K(cyclo[βhF-FΦSRSRQ])-PEG₁₂-OH where PEG₂ may be miniPEG₂.

In embodiments, the delivery construct can be Ac-PKKKRKV-PEG₂-K(cyclo[βhF-FΦGRGRQ])-PEG₂-OH where each PEG₂ may, independently, be miniPEG₂. In embodiments, the delivery construct can be Ac-PKKKRKV-PEG₂-K(cyclo[βhF-FΦGRGRQ])-PEG₁₂-OH where PEG₂ may be miniPEG₂.

In embodiments, the delivery construct can be Ac-PKKKRKV-PEG₂-K(cyclo[FGFGRGRQ])-PEG₂-OH where each PEG₂ may, independently, be miniPEG₂. In embodiments, the delivery construct can be Ac-PKKKRKV-PEG₂-K(cyclo[FGFGRGRQ])-PEG₁₂-OH where PEG₂ may be miniPEG₂.

### Methods of Making

The cargo conjugates and the components of the cargo conjugates can be prepared in a variety of ways known to one skilled in the art of organic synthesis or variations thereon as appreciated by those skilled in the art. The compounds can be prepared from readily available starting materials. Reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4^{th} Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4^{th} Edition); and Larock's Comprehensive Organic Transformations (VCH Publishers Inc., 1989). Other materials, such as the pharmaceutical carriers can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, e.g., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

In embodiments, portions of the cargo constructs can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobomnyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group can be used for the synthesis of the disclosed compounds. Other side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for aspartic acid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl) phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, for example, piperidine, prior to coupling with the α-C-terminal amino acid as described above. One method for coupling to the deprotected 4 (2′,4′-dimethoxyphenyl-Fmoc-aminomethyl) phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, for example, piperidine. Each protected amino acid is then introduced in about 3-fold molar excess, and the coupling can be carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thioanisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivatized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

Polymers, such as PEG groups, can be attached to an oligonucleotide, such as an ASO, a CCCP, EP, or ocular delivery construct under any suitable conditions. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the ASO a cCCP, an EEV, or a compound comprising an EEV (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., α-iodo acetic acid, α-bromoacetic acid, α-chloroacetic acid). If attached to the ASO, a cCCP, an EEV, or a compound comprising an EEV by reductive alkylation, the polymer selected should have a single reactive aldehyde so that the degree of polymerization is controlled. Sce, for example, Kinstler et al., Adv. Drug. Delivery Rev. (2002), 54:477-485; Roberts et al., Adv. Drug Delivery Rev. (2002), 54:459-476; and Zalipsky et al., Adv. Drug Delivery Rev. (1995), 16:157-182.

In order to direct covalently link the AC or linker to the CPP, appropriate amino acid residues of the CPP may be reacted with an organic derivatizing agent that is capable of reacting with a selected side chain or the N- or C-termini of an amino acids. Reactive groups on the peptide or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art.

Methods of making a therapeutic oligonucleotide and conjugating oligonucleotide to linear CPP are generally described in US Pub. No. 2018/0298383, which is herein incorporated by reference for all purposes. The methods may be applied to the cyclic CPPs disclosed herein.

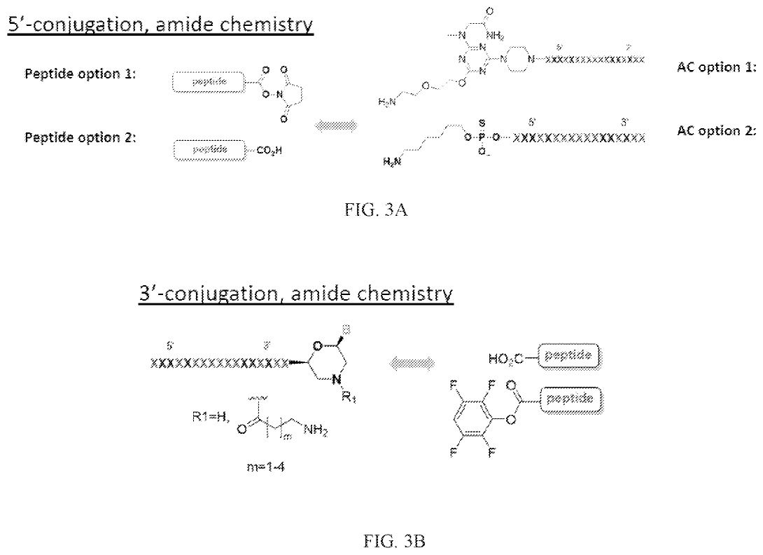

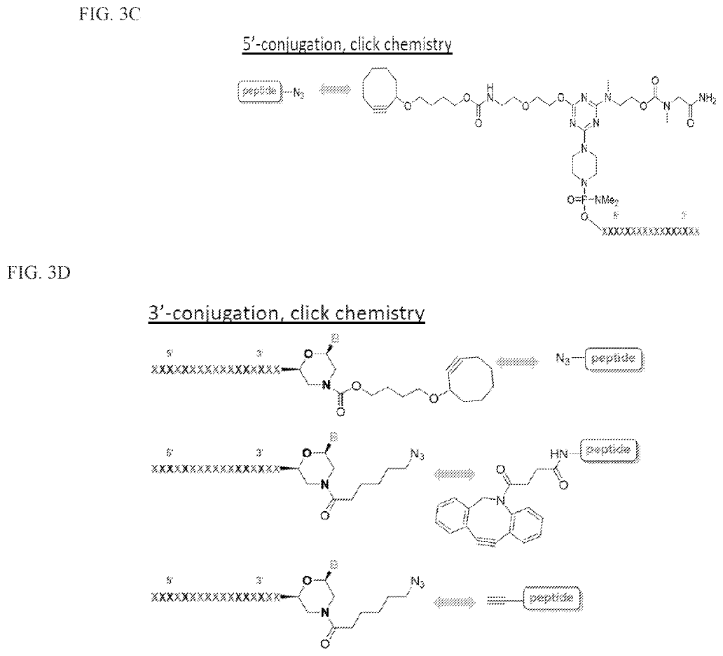

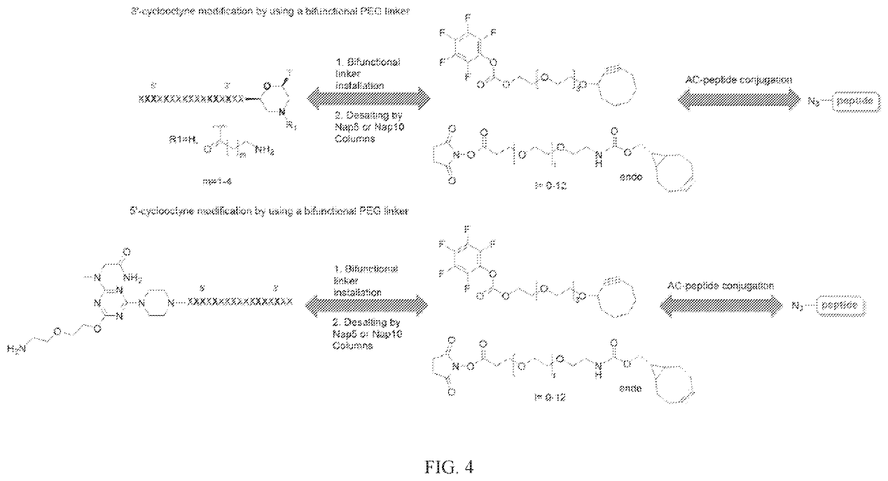

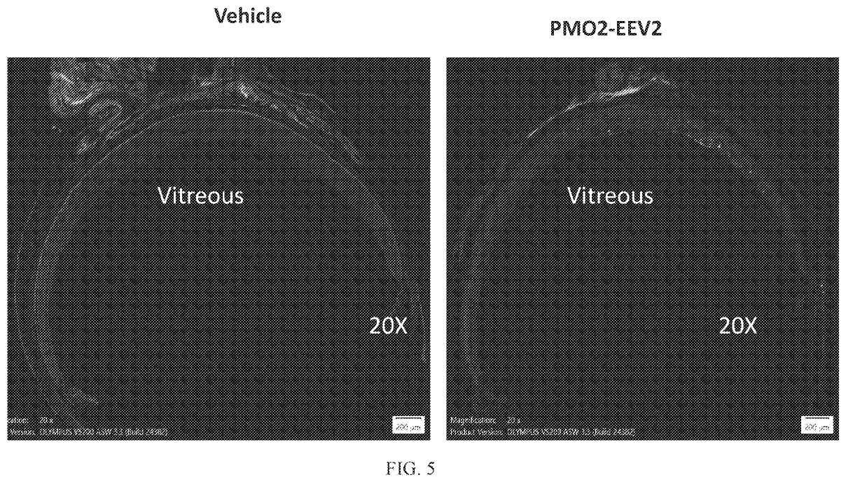

Synthetic schemes are provided in FIGS. 3A-3D and FIG. 4.

Non-limiting examples of compounds that include a CPPs and a reactive group useful for conjugation to an therapeutic moiety, such as an oligonucleotide, a polypeptide, or a small molecule, are shown in Table 3. Example linker groups are also shown. Example reactive groups include tetrafluorophenyl ester (TFP), free carboxylic acid (COOH), an azide (N₃) and an alkyne (e.g., a cyclooctyne). In Table 3, n is an integer from 0 to 20; Pipa6 is AcRXRRBRRXRYQFLIRXRBRXRB wherein B is β-Alanine and X is aminohexanoic acid; Dap is 2,3-diaminopropionic acid; NLS is a nuclear localization sequence; βA is beta alanine; -ss- is a disulfide; PABC is poly(A) binding protein C-terminal domain; Cₓ where x is a number is an alkyl chain of length x; and BCN is bicyclo[6.1.0]nonyne.

TABLE 3
Compounds that include a CPPs and a reactive group

TFP-PEGₙ-K(CPP)
TFP-PEGₙ-K(CPP)-PEGₙ-Dap(palmitoyl)
TFP-PEGₙ-K(CPP)-PEGₙ-Dap(CPP)
TFP-Pip6a
CPP-PEGₙ-TFP
CPP-PEGₙ-K(CPP)-PEGₙ-TFP
CPP-PEGₙ-Lys(N₃)
CPP-K(CPP)-PEGₙ-K(N₃)
CPP-PEGₙ-K(PEGₙ-CPP)-PEGₙ-K(N₃)
CPP-PEGₙ-K(PEGₙ-CPP)-PEGₙ-K(N₃)
CPP-K(CPP)-K(CPP)-PEGₙ-K(N₃)
CPP-PEGₙ-K(PEGₙ-CPP)-K(PEGₙ-CPP)-PEGₙ-K(N₃)
CPP-PEGₙ-K(PEGₙ-CPP)-K(PEGₙ-CPP)-PEGₙ-K(N₃)
Ac-NLS-Lys(CPP)-PEGₙ-K(N₃)
K(N₃)-PEGₙ-NLS-ss-PEGₙ-CPP
BCN-NLS-ss-CPP
CPP-PEGₙ-Val-Cit-PABC-K(N₃)
CPP-PEGₙ-Cys-ss-Cys-K(N₃)
CPP-PEGₙ-Cys-ss-Cys-K(N₃)
CPP-PEGₙ-TFP
CPP-PEGₙ-Lys(N₃)
CPP-PEGₙ-Cys-prodisulfide-K(N₃)
CPP-PEGₙ-K(N₃)
CPP-K(CPP)-PEGₙ-K(N₃)
CPP-PEGₙ-K(CPP)-PEGₙ-TFP
CPP-C₆-TFP
CPP-PEGₙ-K(PEGₙ-CPP)PEGₙ-K(N₃)
Ac-T9-PEGₙ-Lys(CPP-PEGₙ)-K(N₃)
Ac-MSP-PEGₙ-K(CPP-PEGₙ)-K(N₃)
CPP-PEGₙ-TFP
CPP-C₆-TFP
CPP-PEGₙ-K(CPP)-PEGₙ-TFP
CPP-PEGₙ-COOH
CPP-C₁₂-TFP
palmitoyl-PEGₙ-K(CPP)-PEGₙ-TFP
CPP-PEGₙ-K(N₃)
Ac-T9-PEGₙ-K(CPP)-K(N₃)
Ac-MSP-PEGₙ-K(CPP-PEGₙ)-K(N₃)
Ac-NLS-K(CPP)-PEGₙ-K(N₃)
K(N₃)-PEGₙ-NLS-ss-PEGₙ-CPP
K(N₃)-PEGₙ-NLS-K-βA-βA-CPP

In embodiments, the cCPPs have free carboxylic acid groups that may be utilized for conjugation to a therapeutic moiety, such as an oligonucleotide, a polypeptide, or a small molecule. In embodiments, the EEVs have free carboxylic acid groups that may be utilized for conjugation to the therapeutic moiety.

FIGS. 3A-3D and FIG. 4 show example conjugation chemistry schemes. The CPPs of the examples may be modified with linker that have reactive groups allowing conjugation. The linkers in each example scheme may not be fully shown and/or are non-limiting. Two example schemes showing conjugation of a CPP or a modified CPP to the modified 5′ end of an oligonucleotide via an amide bond is shown in FIG. 3A. An example scheme showing the conjugation of a CPP or modified CPP to the modified 3′ end of an oligonucleotide via an amide bond is shown in FIG. 3B. An example scheme showing the conjugation of an azide modified CPP to a 5′-cyclooctyne modified PMO via strain-promoted azide-alkyne cycloaddition is shown in FIG. 3C. Several azide-alkyne conjugation reactions for conjugating a modified 3′ of an oligonucleotide to a modified CPP are shown in FIG. 3D.

Various examples showing conjugation chemistries using a bifunctional compound that includes a PEG moiety and two reactive handles (e.g., activated tetrafluorophenyl ester, alkyne, and N-hydroxysuccinimide ester) are shown in FIG. 4. One of the reactive handles is used to conjugate the AC to the bifunctional molecule and the second reactive handle is used to conjugate the CPP to the bifunctional molecule thereby conjugating the oligonucleotide to the CPP.

### Compositions and Formulations

In embodiments, compositions are provided that include one or more cargo conjugates. In embodiments, the composition is a pharmaceutical composition; that is, a composition designed for administration to a subject.

In embodiments, pharmaceutically acceptable salts and/or prodrugs of the disclosed cargo conjugates are disclosed. Pharmaceutically acceptable salts include salts of the disclosed cargo conjugates that are prepared with acids or bases, depending on the particular substituents found on the cargo conjugates. Under conditions where the cargo conjugates are sufficiently basic or acidic to form stable nontoxic acid or base salts, administration of the cargo conjugates as salts can be appropriate. Examples of pharmaceutically acceptable base addition salts include sodium, potassium, calcium, ammonium, or magnesium salt. Examples of physiologically acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a cargo conjugate can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

In vivo application of the disclosed cargo conjugates, and compositions containing them, can be accomplished by any suitable method and technique presently or prospectively known to those skilled in the art. For example, the disclosed cargo conjugates can be formulated in a physiologically- or pharmaceutically-acceptable form and administered by any suitable route known in the art including, for example, compositions for dropping on the eye or for injecting into the eye (intraocular administration). Administration of the disclosed cargo conjugates or compositions can be a single administration, or at continuous or distinct intervals as can be readily determined by a person skilled in the art.

The cargo conjugates, and compositions comprising them, can also be administered utilizing liposome technology. These delivery methods can, advantageously, provide a uniform dosage over an extended period of time. The cargo conjugates can also be administered in their salt derivative forms or crystalline forms.

The cargo conjugates can be formulated according to known methods for preparing pharmaceutically acceptable compositions (pharmaceutical compositions). Formulations are described in detail in a number of sources which are well known and readily available to those skilled in the art. For example, Remington's Pharmaceutical Science by E. W. Martin (1995) describes formulations that can be used in connection with the disclosed methods. In general, the cargo conjugates can be formulated such that an effective amount of the cargo conjugates is combined with a suitable carrier in order to facilitate effective administration of the cargo conjugates. The compositions used can also be in a variety of forms. These include, for example, liquid dosage forms, such as liquid solutions or suspension, and injectable and infusible solutions. The form depends on the intended mode of administration and therapeutic application. The compositions can also include conventional pharmaceutically-acceptable carriers and diluents which are known to those skilled in the art. Examples of carriers or diluents for use with the compounds include water, ethanol, dimethyl sulfoxide, glycerol, alumina, starch, saline, and equivalent carriers and diluents. To provide for the administration of such dosages for the desired therapeutic treatment, compositions can advantageously comprise between about 0.1% and 100% by weight of the total of one or more of the subject cargo conjugates based on the weight of the total composition including carrier or diluent.

Formulations suitable for administration include, for example, aqueous sterile injection solutions, which can contain antioxidants, buffers, bacteriostats, and solutes that render the formulation isotonic with the interstitial fluid of the eye of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients mentioned above, the compositions disclosed herein can include other agents conventional in the art having regard to the type of formulation in question.

Cargo conjugates, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions to a cell can comprise attaching the cargo conjugates to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Cargo conjugates can also be incorporated into polymers, examples of which include poly(D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of an ocular disease, the cargo conjugate can be administered to a patient in need of treatment in combination with other active agents designed to treat the disease. These other substances or treatments can be given at the same as or at different times from the cargo conjugates. For example, a pharmaceutical composition comprising a cargo conjugate can further include a second active agent designed to treat the disease.

Cargo conjugates and compositions containing the same, including pharmaceutically acceptable salts or prodrugs thereof, can be administered by drop, infusion, or injection. In embodiments, the compounds and compositions can be administered parenterally. In embodiments, the compounds and compositions can be administered intravenously. In embodiments, the compounds and compositions can be administered subcutaneously. In embodiments, the compounds and compositions disclosed herein can be administered intraocularly. In embodiments, the compounds and compositions can be administered intravitreally. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. Isotonic agents can be included, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a cargo conjugate and/or other agent in the required amount in the appropriate solvent with various other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

For topical administration, cargo conjugates and compositions containing the same can be applied in as a liquid or solid. However, it will generally be desirable to administer them topically to the eye as compositions, in combination with a ophthalmologically acceptable carrier, which can be a solid or a liquid. In embodiments, compounds are formulated into a solution or suspension for topical application to a surface of the eye, such as to a surface of the cornea.

Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to modify the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the surface of the eye.

Also disclosed are pharmaceutical compositions that comprise a compound disclosed herein in combination with a pharmaceutically acceptable carrier, including, but not limited to, pharmaceutical compositions adapted for oral, topical, parenteral or intraocular administration, comprising an amount of a compound. The dose administered to a patient, particularly a human, should be sufficient to achieve a therapeutic response in the patient over a reasonable time frame, without lethal toxicity, and causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a cargo conjugate or composition containing the same in one or more containers. The disclosed kits can optionally include pharmaceutically acceptable carriers and/or diluents. A kit can include one or more other components, adjuncts, or adjuvants. A kit includes one or more additional active agents for treating an eye disease. A kit can include instructions or packaging materials that describe how to administer a cargo conjugate or composition of the kit. Containers of the kit can be of any suitable material, e.g., glass, plastic, metal, etc., and of any suitable size, shape, or configuration. A cargo conjugate can be provided in the kit as a solid, such as a tablet, pill, or powder form. A cargo conjugate can be provided in the kit as a liquid or solution. A kit can comprise an ampoule or syringe containing a cargo conjugate or composition containing the same in liquid or solution form.

### Methods of Use

Also provided herein are methods of use of the cargo conjugates or a pharmaceutical compositions comprising one or more cargo conjugates. The method comprises identifying a subject having a disease or disorder of the eye and administering a cargo conjugate or a composition comprising one or more cargo conjugates to the subject. The cargo conjugate includes a therapeutic moiety designed to treat an ocular disease. As such, the method may be a method of treatment of an ocular disease.

Identifying a subject includes identifying a subject that has an ocular disease. The subject may be a mammal. The subject may be human. A subject that has an ocular disease may display one or more symptoms or clinical signs associated with an ocular disease; may have a genetic signature associated with an ocular disease, for example, may have a mutation in a gene associated with an ocular disease; or both.

Identification of a subject having an ocular disease may include diagnosing the subject with an ocular disease. Diagnosis of an ocular disease may be by way of a physician or other health care provider conducting tests and exams to identify a cause of symptoms displayed by a subject.

Genetic testing may be used to diagnose a subject with an ocular disease. Genetic testing may include sequencing a portion of the subjects genome to identify genetic anomalies in one or more genes associated with an ocular disease. To identify genetic anomalies, the sequenced portion of a subject's genome can be compared to a standard genome that does not contain genetic anomalies in the genes associated with the eye. Sequencing technologies and analysis methods are well known.

Additionally, genetic testing following diagnosis of an ocular disease may be used to identify a particular genetic anomaly causing the disease. Identification of the particular genetic anomaly, such as a mutation that results in the displayed phenotype, may allow one to choose a particular therapeutic moiety designed to have a biological effect given the genetic anomaly. For example, some therapeutic moieties are designed to be effective when a subject displays a particular genetic anomaly of a disease and may not be effective or as effective if the subject displays a different anomaly of the same disease. For example, a subject diagnosed with an ocular disease characterized by one or both of two independent mutations may undergo genetic testing to determine if they have the first mutation, the second mutation, or both. If the subject has the first mutation, a first cargo conjugate having a first therapeutic moiety designed to be effective in a disease state of the first mutation may be administered to the subject. If the subject has the second mutation, a second cargo conjugate having a second therapeutic moiety designed to be effective in a disease state of the second mutation may be administered to the subject. The first cargo conjugate may not be effective or as effective if the subject displays the second mutation and the second cargo conjugate may not be effective or as effective if the subject displays the first mutation.

The method of administration further includes administering a cargo conjugate or a composition comprising one or more cargo conjugates to the subject. In embodiments, the cargo conjugate or a pharmaceutical composition directly to the eye of the subject; that is, the cargo conjugate or pharmaceutical composition is ocularly administered to the subject. In embodiments, the composition is administered directly to a tissue, such as, for example, to the cornea, sclera, lens, iris, ciliary body, optic nerve, choroid, or retina.

In embodiments, the cargo conjugate or pharmaceutical composition is topically administered. In embodiments, the cargo conjugate or pharmaceutical composition is delivered to the cornea. For example, the pharmaceutical composition may be an eye drop formulation that is contacted with the cornea of the subject's eye. Other routes of topical administration include drug delivery contacts soaked or coated with a cargo conjugate or pharmaceutical composition. The contact may be designed for and allow a delayed and/or sustained release of the cargo conjugate.

In embodiments, the cargo conjugate or pharmaceutical composition is periocularly administered. Periocular administration includes drug deposition on the scleral external surface. The drug can diffuse through the scelara. Examples of periocular administration include, but are not limited to, subconjunctival administration, subTenon injection, retrobulbar administration, peribulbar juxtascleral administration, posterior juxtascleral administration, and iontophoresis techniques such as the EYEGATE II system from Kiora Pharmaceuticals, Inc. (Encinitas, CA).

In embodiments, the cargo conjugate or pharmaceutical composition is intravitreally delivered. Intravitreal injection delivers the injected species into the vitreous humor. Techniques that may be used for intravitreal administration include intravitreal injection (also called intraocular injection) and intravitreal infusion.

In embodiments, the cargo conjugate or pharmaceutical composition is subretinally administered. Subretinal administration includes delivery of drug or composition to the retina. Examples of subretinal administration include retinal injection (subretinal injection) and implantation of biodegradable drug delivery devices or non-biodegradable medical devices. Subretinal administration may involve subjecting the subject to a pars plana vitrectomy and a retinotomy. Subretinal administration may deliver the drug or composition to the space between or including the retinal pigment epithelium layer and the photoreceptor layer.

In embodiments, the cargo conjugate or pharmaceutical composition is suprachoroidally administered. Suprachoroidal administration involves depositing a drug or composition in the suprachoroidal space between the scelara and the choroid. Techniques that may be used for suprachoroidal administration include injection and infusion using, for example, a micro needle, a cannulas, or a catheter.

In embodiments, the cargo conjugate or pharmaceutical composition is intracamerally administered. Intracameral administration involves depositing a drug or composition into the anterior chamber of the eye. Intracameral administration may be accomplished by intracameral injection.

In embodiments, the cargo conjugate or pharmaceutical composition is delivered to the eye through the use of a medical device. The medical device may be biodegradable or non-biodegradable. The medical device may be implanted at any location within the eye. For example, a cargo conjugate or pharmaceutical composition may be a part of an intravitreal implant, a Cul-de-sac implant, or an episcleral implant.

Therapeutically effective dosages of the cargo conjugates and pharmaceutical compositions can be determined by comparing their in vitro activity, and in vivo activity in animal models. Methods for the extrapolation of effective dosages in mice, and other animals, to humans are known to the art.

Therapeutically effective dosage ranges for the administration of the cargo conjugates and compositions containing the same are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### Certain Definitions

The type and origin of the molecule are not limited by the case and/or typeface (e.g., bold, italics, etc.). A specific name of gene, transcript, or protein may be denoted as unitalicized and upper case, italicized and upper case, unitalicized and upper case, or unitalicized and lower case. The specific type and origin of the molecule are to be understood in the context of the use of the stated molecule.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, the term “cyclic cell penetrating peptide” or “cCPP” refers to a peptide that facilitates the delivery of a cargo, e.g., a therapeutic moiety, into a cell.

As used herein, the term “endosomal escape vehicle” (EEV) refers to an ocular delivery construct comprising a cCPP, an exocyclic peptide (EP), and a linker.

As used herein, the term “EEV-conjugate” refers to an endosomal escape vehicle defined herein conjugated by a chemical linkage (i.e., a covalent bond or non-covalent interaction) to a cargo. The cargo can be a therapeutic moiety (e.g., an oligonucleotide, peptide or small molecule) that can be delivered into a cell by the EEV.

As used herein, the term “exocyclic peptide” (EP) refers to two or more amino acid residues linked by a peptide bond that can be conjugated to a cyclic cell penetrating peptide (cCPP), a linker, a therapeutic moiety, or any combination thereof. The EP, when a part of an ocular delivery construct, may alter the tissue distribution and/or retention of the compound. Typically, the EP comprises at least one positively charged amino acid residue, e.g., at least one lysine residue and/or at least one arginine residue. Non-limiting examples of EP are described herein. The EP can be a peptide that has been identified in the art as a “nuclear localization sequence” (NLS).

As used herein, “linker” or “L” refers to a moiety that covalently couples two or more components of a delivery construct and/or a cargo construct; includes one or more functional groups that can be used to conjugate one or more components to the linker; or both. For example, a linker can covalently couple one or more moieties (e.g., an exocyclic peptide (EP) and a cargo; a cCPP and an EP; a cCPP and a cargo; a cCPP, an EP, and a cargo) to the cyclic cell penetrating peptide (cCPP). A linker can covalently link a cargo and a delivery construct and/or two or more components of a delivery construct. The linker can comprise a natural or non-natural amino acid or polypeptide. The linker can be a synthetic compound containing two or more appropriate functional groups suitable to conjugate one or more components of a delivery construct and/or a delivery construct to a cargo. The linker can comprise a polyethylene glycol (PEG) moiety. The linker can comprise one or more amino acids. The cCPP may be covalently bound to a cargo via a linker.

As used herein, the term “oligonucleotide,” “nucleic acid,” and “polynucleotide” refer to an oligomeric compound comprising a plurality of linked nucleotides or nucleosides. One or more nucleotides of an oligonucleotide can be modified. An oligonucleotide can comprise ribonucleic acid (RNA) or deoxyribonucleic acid (DNA). Oligonucleotides can be composed of natural and/or modified nucleobases, sugars and covalent internucleoside linkages, and can further include non-nucleic acid conjugates.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins and the like.

The term “therapeutic agent” or “therapeutic moiety” can be used to refer to a cargo that has therapeutic, prophylactic or other biological activity. The therapeutic agent can be a peptide, oligonucleotide or a small molecule. The therapeutic agent can be an enzyme. The therapeutic agent can be an antibody or antigen binding fragment. The therapeutic agent can be an oligonucleotide. The therapeutic agent can be an antisense oligonucleotide. The therapeutic agent can be one or more components of a gene editing machinery (GEM). The therapeutic agent can be an oligonucleotide encoding one or more components of a gene editing machinery (GEM). The therapeutic agent can include gRNA. The therapeutic agent can include a nuclease or an oligonucleotide encoding a nuclease. In embodiments, the nuclease is a Cas nuclease. In embodiments, the nuclease is a Cas9 nuclease. The therapeutic agent can include a ribonucleoprotein (RNP) or an oligonucleotide encoding a RNP.

The term “small molecule” refers to an organic compound with pharmacological activity and a molecular weight of less than about 2000 Daltons, or less than about 1000 Daltons, or less than about 500 Daltons. Small molecule therapeutics are typically manufactured by chemical synthesis.

As used herein, the term “contiguous” refers to two amino acids, which are connected by a covalent bond. For example, in the context of a representative cyclic cell penetrating peptide (cCPP) such as

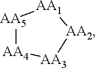

AA₁/AA₂, AA₂/AA₃, AA₃/AA₄, and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic cell penetrating peptides (cCPP) can have amino acids (e.g., arginine) incorporated therein through formation of one or more peptide bonds. The amino acids incorporated into the cCPP may be referred to residues, or simply as an amino acid. Thus, arginine or an arginine residue refers to

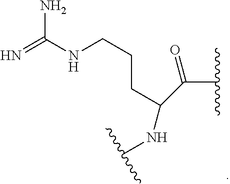 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

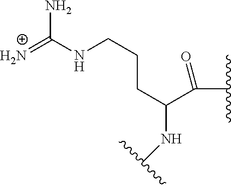 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. The chiral molecule can be an amino acid residue having a “D” and “L” enantiomer. Molecules without a chiral center, such as glycine, can be referred to as “achiral.”

As used herein, the term “hydrophobic” refers to a moiety that is not soluble in water or has limited solubility in water. Generally, neutral moieties and/or non-polar moieties, or moieties that are predominately neutral and/or non-polar are hydrophobic. Hydrophobicity can be measured by one of the methods disclosed herein.

As used herein “aromatic” refers to an unsaturated cyclic molecule having 4n+2π electrons, wherein n is any integer. The term “non-aromatic” refers to any unsaturated cyclic molecule which does not fall within the definition of aromatic. For example, any linear, branched or cyclic molecule which does not fall within the definition of aromatic is non-aromatic. Examples of non-aromatic amino acids include, but are not limited to, glycine and citrulline.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₈ alkyl. A C₁-C₈ alkyl includes C₅ alkyls, C₄ alkyls, C₃ alkyls, C₂ alkyls and C₁ alkyl (i.e., methyl). A C₁-C₆ alkyl includes all moieties described above for C₁-C₈ alkyls but also includes C₆ alkyls. A C₁-C₁₀ alkyl includes all moieties described above for C₁-C₈ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, i-butyl, n-pentyl, t-amyl, n-hexyl, n-heptyl, n-octyl, n-nonyl, n-decyl, n-undecyl), and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, S-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group-OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups-C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where Rₐ and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems, and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR₂SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

As used herein, by a “subject” is meant an individual. The subject can be a mammal. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to reducing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the compound or composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to reduce any degradation of the active ingredient and any adverse side effects in the subject.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

“Amino acid” refers to an organic compound that includes an amino group and a carboxylic acid group and has the general formula

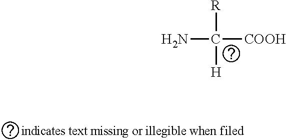

where R can be any organic group. An amino acid may be a naturally occurring amino acid or non-naturally occurring amino acid. An amino acid may be a proteogenic amino acid or a non-proteogenic amino acid. An amino acid can be an L-amino acid or a D-amino acid. The term “amino acid side chain” or “side chain” refers to the characterizing substituent (“R”) bound to the α-carbon of a natural or non-natural α-amino acid. An amino acid may be incorporated into a polypeptide via a peptide bond.

As used herein, an “uncharged” amino acid is an amino acid having a side chain that has a net neutral charge at pH 7.35 to 7.45. Examples of uncharged amino acids include, but are not limited to, glycine and citrulline.

As used herein, a “charged” amino acid is an amino acid having a side chain having a net charge at a pH of 7.35 to 7.45. An example of a charged amino acid is arginine.

As used herein, “polyethylene glycol” and “PEG” are used interchangeably. “PEGm,” and “PEGₘ,” are, or are derived from, a molecule of the formula HO(CO)—(CH2)ₙ—(OCH₂CH₂)ₘ—NH₂ where n is any integer from 1 to 5 and m is any integer from 1 to 23. In embodiments, n is 1 or 2. In embodiments, n is 1. In embodiments, n is 2. In embodiments, n is 1 and m is 2. In embodiments, n is 2 and m is 2. In embodiments, n is 1 and m is 4. In embodiments, n is 2 and m is 4. In embodiments, n is 1 and m is 12. In embodiments, n is 2 and m is 12.

As used herein, “miniPEGm” or “miniPEGₘ” are, or are derived from, a molecule of the formula HO(CO)—(CH₂)ₙ—(OCH₂CH₂)ₘ—NH₂ where n is 1 and m is any integer from 1 to 23. For example, “miniPEG2” or “miniPEG₂” is, or is derived from, (2-[2-[2-aminoethoxy]ethoxy]acetic acid), and “miniPEG4” or “miniPEG₄” is, or is derived from, HO(CO)—(CH₂)ₙ—(OCH2CH₂)ₘ—NH₂ where n is 1 and m is 4. “2-[2-[2-aminoethoxy]ethoxy]acetic acid” is also referred to as AEEA, miniPEG or PEG₂.

As used herein, the term “target” refers to a macromolecule associated with a disease. In embodiments, the target is a macromolecule implicated in a disease or pathology of the eye. In embodiments, the target is a polypeptide or protein. In embodiments, the target is an oligonucleotide. In embodiments, the oligonucleotide target comprises DNA. In embodiments, the oligonucleotide target comprises RNA. In embodiments, the oligonucleotide target comprises mRNA. In embodiments, the target is associated with an ocular disease.

As used herein, the terms “targeting” or “targeted to” refer to selective association of a therapeutic moiety with a target molecule. In embodiments, an antisense oligonucleotide may selectively bind with a target nucleic acid molecule or a region of a target nucleic acid molecule. In embodiments, a peptide may selectively bind with a target protein or region of a target protein. In embodiments, selective binding of a therapeutic moiety with a target molecule is useful for the treatment of a disease, pathologies or other abnormal states or conditions of the eye. In embodiments, the therapeutic moiety includes an antisense oligonucleotide that is capable of hybridizing to a target nucleic acid under physiological conditions. In embodiments, the antisense compound targets a specific portion or site within the target nucleic acid, for example, a portion of the target nucleic acid having at least one identifiable structure, function, or characteristic such as a particular exon or intron, or selected nucleobases or motifs within an exon or intron.

As used herein” selectively binds” or “specifically binds” means that a therapeutic moiety exhibits enhanced binding to a target molecule as compared to another molecule. With reference to the interaction between an oligonucleotide therapeutic moiety and an oligonucleotide target, a therapeutic oligonucleotide is “specific” for a target oligonucleotide if the therapeutic oligonucleotide has at least 90%, at least 95%, at least 96%, at least 97%, at least 98%, at least 99% or 100% sequence identity with a portion of the nucleic acid sequence of the target oligonucleotide when the nucleic acid sequences of the therapeutic oligonucleotide and the target oligonucleotide are aligned. A therapeutic oligonucleotide that is “selectively binds” or “specifically binds” to a target oligonucleotide is one that is capable of hybridizing to the target oligonucleotide of interest and not substantially hybridizing to other oligonucleotide sequences which are not of interest under stringent hybridization/washing conditions. An oligonucleotide which is “gene-specific” is specific for a target oligonucleotide sequence in a gene of interest and does not substantially hybridize to other genes.

As used herein, “hybridize” or “specifically hybridize” refers to a process where two complementary nucleic acid strands anneal to each in accordance with Watson-Crick base pairing rules. Hybridizations are typically and preferably conducted with probe-length nucleic acid molecules, preferably 20-100 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to determine the appropriate stringency of hybridization/washing conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

With reference to the interaction of an antibody or antigen-binding fragment thereof, or a binding protein, “selectively binds” or “specifically binds” or “specific binding” means that the interaction is dependent upon the presence of a particular structure (e.g., an antigenic determinant or epitope) on the molecule. In embodiments, a binding protein or antibody or antigen-binding fragment thereof specifically binds to a target with a KD greater than 10⁻⁶M, for example, with a KD between 10⁻⁶M and 10⁻¹²M.

As used herein, the terms “target nucleic acid sequence,” “target polynucleotide sequence,” and “target nucleotide sequence” refer to the nucleic acid sequence or the nucleotide sequence to which a therapeutic moiety, such as an antisense oligonucleotide, binds or hybridizes. Target nucleic acids include, but are not limited, to a portion of a target transcript, target RNA (including, but not limited to pre-mRNA and mRNA or portions thereof), as well as a portion of target non-translated RNA, such as miRNA. In embodiments, a target nucleic acid can be a portion of a target cellular gene (or mRNA transcribed from such gene) whose expression is associated with a particular disorder or disease state. The term “portion” refers to a defined number of contiguous (i.e., linked) nucleotides of a nucleic acid.

As used herein, the term “transcript” or “gene transcript” refers an RNA molecule transcribed from DNA and includes, but is not limited to mRNA, pre-mRNA, and partially processed RNA.

The terms “target transcript” and “target RNA” refer to the pre-mRNA or mRNA transcript that is bound by the therapeutic moiety. The target transcript may include a target nucleotide sequence. In embodiments, the target transcript includes a splice site. In embodiments, the target RNA includes a polyadenylation site or a portion thereof.

The term “target gene” and “gene of interest” refer to the gene of which modulation of the expression and/or activity is desired or intended. The target gene may be transcribed into a target transcript that includes a target nucleotide sequence. The target transcript may be translated into a protein of interest.

The term “target protein” refers to the polypeptide or protein encoded by the target transcript (e.g., target mRNA).

As used herein, the term “expression,” “gene expression,” “expression of a gene,” or the like refers to all the functions and steps by which information encoded in a gene is converted into a functional gene product, such as a polypeptide or a non-coding RNA, in a cell. Examples of non-coding RNA include transfer RNA (IRNA) and ribosomal RNA. Gene expression of a polypeptide includes transcription of the gene to form a pre-mRNA, processing of the pre-mRNA to form a mature mRNA, translocating the mature mRNA from the nucleus to the cytoplasm, translation of the mature mRNA into the polypeptide, and assembly of the encoded polypeptide. Expression includes partial expression. For example, expression of a gene may be referred to herein as generation of a gene transcript. Translation of a mature mRNA may be referred to herein as expression of the mature mRNA.

As used herein, “modulate” or “modulation” refers to an increase (e.g., upregulation) or decrease (e.g., downregulation) of an activity of interest. As used herein, “modulation of gene expression” refers to modulation of one or more of the processes associated with gene expression. For example, modification of gene expression may include modification of one or more of gene transcription, RNA processing, RNA translocation from the nucleus to the cytoplasm, and translation of mRNA into a protein. As used herein, “modulation of protein activity” refers to modulation of protein activity for example, by increasing or decreasing protein expression, increasing or decreasing expression of a fragment of a protein having a biological activity, or by blocking one or more protein-protein or protein-substrate interactions.

All publications, patents and patent applications mentioned in the specification are indicative of the level of skill of those skilled in the art to which this invention pertains. All publications, patents and patent applications are herein incorporated by reference to the same extent as if each individual publication or patent application was specifically and individually indicated to be incorporated by reference.

### EXAMPLES

The present disclosure is illustrated by the following examples. It is to be understood that the particular examples, materials, amounts, and procedures are to be interpreted broadly in accordance with the scope and spirit of the disclosure as set forth herein.

The following examples illustrate, among other things, that the EEVs described herein may effectively deliver cargo (e.g., a therapeutic moiety) intracellularly when delivered directly to the eye. The examples also illustrate that an EEV-PMO conjugate may achieve broad biodistribution throughout retina after intravitreal injection, including delivery to photoreceptors.

Abbreviations for reagents used in the Examples are provided as follows HATU (1-[Bis (dimethylamino)methylene]-1H-1,2,3-triazolo[4,5-b]pyridinium 3-oxide hexafluorophosphate); DMSO (dimethylsulfoxide); DIPEA (N,N-Diisopropylethylamine); and DAPI (4′,6-diamidino-2-phenylindole).

### Example 1: Delivery of Cyanine-5 (Cy5) to the Retina

In this example, C57Bl/6 mice were injected intravitreally with vehicle, EEV conjugated to cyanine-5 (Cy5; a red fluorescent dye), Cy5 alone or no injection.

Table 4 provides a summary of the experimental protocol used to evaluate EEV (EEV1) delivery of Cy5 to the eye.

TABLE 4
Summary of Experimental Protocol

| | | | Composition injected | |
|---|---|---|---|---|
| Mouse | Age (weeks) | Weight (g) | Left eye | Right eye |
| A1 | 5.6 | 23.26 | Vehicle | EEV1-Cy5 |
| A2 | 5.6 | 24.21 | Vehicle | EEV1-Cy5 |
| A3 | 5.3 | 21.2 | Vehicle | EEV1-Cy5 |
| B1 | 5.6 | 24.27 | Un-injected | Cy5 |
| B2 | 5.3 | 22.94 | Un-injected | Cy5 |
| B3 | 5.3 | 18.92 | Un-injected | Cy5 |

As shown in Table 4, compositions comprising vehicle, Cy5, or EEV1-Cy5 were injected into the retina of the left and/or right eyes of six C57BL/6 male mice. After 24 hours, the eyes were harvested, embedded, sectioned, and imaged for Cy5 (red) fluorescence and counterstained with DAPI (blue). No detectable signal above background was detected in untreated, vehicle, and Cy5 groups (data not shown), Distribution throughout the inner and outer nuclear layer, and in one animal retinal pigment epithelium (RPE) was observed for the EEV1-Cy5 group, demonstrating that the EEV effectively intracellularly delivered the Cy5.

The structure of the EEV1 was Ac-PKKKRKV-K(cyclo[FfΦGrGrQ])-PEG₁₂-K(N₃)—NH₂). Cy5-DBCO (Cy5-azadibenzocyclooctyne) was conjugated to EEV1 using strain-promoted azide-alkyne cycloaddition, purified via reverse-phase HPLC and lyophilized.

### Example 2. Delivery of Oligonucleotide to the Retina

Experiments were also performed using (i) a different EEV (EEV2) conjugated to an oligonucleotide (PMO1) and to Cy5 and (ii) the PMO conjugated to Cy5. The structure of EEV2 was as follows: Ac-PKKKRKV-miniPEG₂-K(cyclo[FGFGRGRQ]-PEG₁₂-OH. PMO1 had a nucleotide sequence of 5′-ATATTGCTATTACCTTAACCCAGAA-3′. The 3′ end of the PMO1 was attached to the linker (PEG₁₂-OH) of EEV2 via amide conjugation. Briefly, BEV2 and PMO1 were dissolved in DMSO, then predissolved HATU and DIPEA were added, and the solution was allowed to react at room temperature (RT) until complete. After the reaction was complete, the reaction was quenched with water/acetonitrile before purification and subsequent lyophilization. The Cy5-DBCO was conjugated to the 5′ end of the PMO using strain-promoted alkyne-azide cycloaddition using standard protocols, followed by purification and lyophilization.

For the PMO without the EEV2, Cy5-DBCO was attached to the 5′ end of the PMO using strain-promoted alkyne-azide cycloaddition using standard protocols, followed by purification and lyophilization.

The EEV2-PMO-Cy5 and PMO-Cy5 formulation was injected into the retinas of the mice. No detectable signal above background was detectable in vehicle and Cy5 groups (data not shown). Weak distribution was observed into ganglion cell layer (GCL)/inner nuclear layer (INL) for PMO-Cy5. Broad distribution was observed throughout the retina for EEV2-PMO-Cy5, demonstrating that EEV2 effectively intracellularly delivered the PMO when injected into the eye.

### Example 3: Biodistribution of an Oligonucleotide in the Retina

Similar to Example 2, C57Bl/6 mice were injected via bilateral intravitreally injection with vehicle, or 25 μg an EEV conjugated to a PMO designed to induce exon skipping in mRNA produced in retinal cells. The EEV used was EEV2 (Ac-PKKKRKV-miniPEG₂-K(cyclo[FGFGRGRQ]-PEG₁₂-OH). Similar to Example 2, the 3′ end of the PMO was attached to the linker (PEG₁₂-OH) of EEV2 via amide conjugation. Seven days after injection, the mouse eyes were for histology analyzation.

Harvested eyes were stained with DAPI (blue) and a rabbit anti-PMO antibody (green).

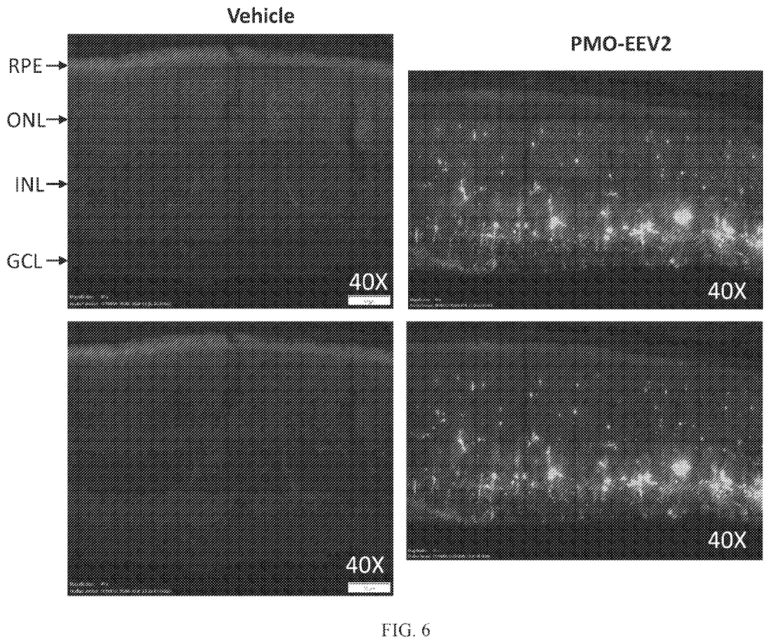

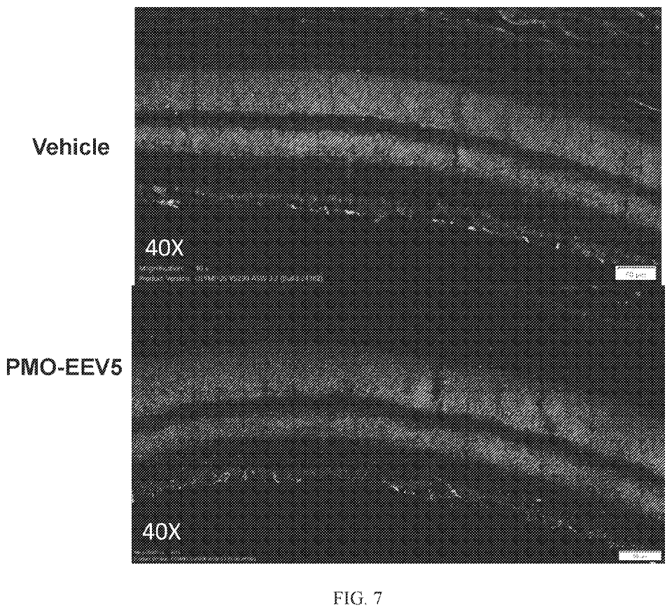

The anti-PMO antibody was raised by immunizing a rabbit with a PMO and conducting downstream development using known methods. The stains clearly show the retinal pigment epithelium (RPE), the outer nuclear layer of the retina (ONL), the inner nuclear layer of the retina (INL), and the ganglion cell layer (GCL) of the retina (FIG. 5; see FIG. 1B for a schematic of the layers of the retina). The PMO-EEV2 conjugate was localized to the retina (FIG. 5, top). Focusing in on the layers of the retina, the PMO-EEV2 conjugate was distributed in the outer nuclear lever of the retina, the inner nuclear layer of the retina, and the ganglion cell layer of the retina as well as the layers between those explicitly labeled (not all retinal layers could be discreetly determined with the staining method used; FIG. 6). Since EEV2 was able to deliver a PMO to the retina, EEV2 may be useful for delivering PMOs for treating various ocular diseases associated with the retina.

Another experiment was conducted to analyze the ability of the PMO-EEV2 to induce exon skipping in a gene expressed in the retina. Compared to a PMO only control, exon skipping was observed for cells treated with the PMO-EEV2 conjugate. This indicates that EEV2 is able to penetrate cells of the retina and deliver a PMO to the cytosol.

### Example 5: Evaluation of in Vivo Tolerability of an PMO-EEV5 Conjugate

Similar to Example 2, mice were injected via intravitreally injection with vehicle, or an EEV conjugated to a PMO designed to induce exon skipping in mRNA produced in retinal cells The EEV used was EEV5 (Ac-PKKKRKV-miniPEG₂-K(cyclo[FfFGRGRQ])-miniPEG₂-K(N₃)—NH₂). The 3′ end of the PMO was attached to the linker (K(N₃)) of EEV5 via click chemistry. Seven days after injection, the mouse eyes were harvested and sectioned for histology analyzation.

FIG. 7 shows the results. Eye tissue treated with DAPI and a GFAP dye. DAPI stains nuclei. The GFAP dye stains glial fibrillary acidic protein, a protein in astrocytes. Intravitreal dosing of the PMO-EEV5 conjugate was well tolerated in mice with no evidence in changes in retinal structure. Additionally, lack of astrocyte infiltration into the retina suggests no inflammation after treatment.

### Example 5: Evaluation of Various Delivery Constructs Conjugated to a PMO

Six delivery constructs were conjugated to a PMO designed to induce exon skipping in a gene associated with a retinal disease. The PMO was conjugated to various delivery constructs using amide chemistry when the delivery construct included a terminal carboxylic acid group (-PEGx-OH) or click chemistry when the delivery construct included a terminal azide (N₃). The structure of the delivery constructs (EEVs) is provided in Table 5 below.

C57BL/6 mice were injected via bilateral intravitreally injection with vehicle, 25 μg of a control, PMO, or PMO conjugated with different EEVs (EEV2-8 listed in Table 5). Each treatment group included 4 mice (8 eyes). The control was an ASO (not a PMO) known to induce exon skipping the gene targeted by the PMO. Seven days post injection, mouse eyes were harvested, exon skipping in six eyes was evaluated by RT-PCR.

TABLE 5
EEV Constructs

| No. | Structure |
|---|---|
| EEV2 | Ac-PKKKRKV-miniPEG₂-K(cyclo[FGFGRGRQ]- |
| | PEG₁₂-OH |
| EEV3 | cyclo[Ff-Nal-RrRrQ]-PEG₁₂-OH |
| EEV4 | Ac-PKKKRKV-miniPEG-K(cyclo[GfFGrGrQ])- |
| | PEG₂-K(N₃)—NH₂ |
| EEVS | Ac-PKKKRKV-miniPEG-K(cyclo[FfFGRGRQ])- |
| | PEG₂-K(N₃)—NH₂ |
| EEV6 | Ac-PKKKRKV-miniPEG-K(cyclo[Ff-Nal- |
| | GrGrQ])-PEG₁₂-OH |
| EEV7 | Ac-PKKKRKV-miniPEG-K(cyclo[FGFRRRRQ])- |
| | PEG₁₂-OH |

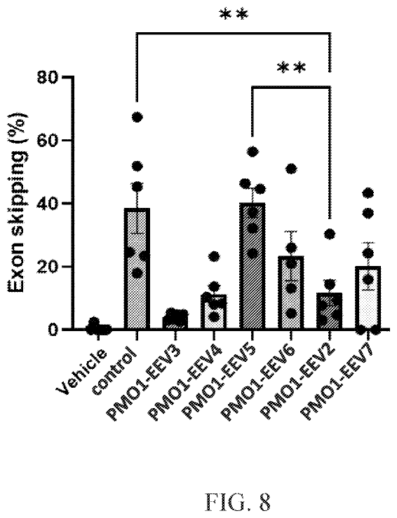

FIG. 8 shows the results. Conjugates having the delivery constructs EEV5, EEV6, and EEV7 induced the greatest amount of exon skipping. These results indicate a number of delivery constructs described herein are effective in delivering a PMO to appropriate cells in the eye to induce exon skipping in a gene expressed in the retina, suggesting that the delivery constructs may also be suitable for use in delivering other cargo to cells of the eye for treating ocular diseases.

### Example 6: Evaluation of Various Delivery Constructs Conjugated to a PMO

Delivery constructs can be conjugated to cargo that includes therapeutic moiety such as a PMO designed to modulate expression of a gene associated with an ocular disease. The cargo can be conjugated to various delivery constructs using amide chemistry when the delivery construct includes a terminal carboxylic acid group (-PEGx-OH) or click chemistry when the delivery construct includes a terminal azide (N₃). The structure of the delivery constructs (EEVs) that may be tested are provided in Table 6 below.

Similar to Example 5, C57BL/6 mice may be injected via bilateral intravitreally injection with vehicle, 25 μg of a control, PMO, or PMO conjugated with different EEVs Table 6). A control may be included, for example, an ASO (not a PMO) known to induce exon skipping the gene targeted by the therapeutic moiety. At a suitable time post injection, mouse eyes may be harvested and the impact of the therapeutic moiety evaluated.

TABLE 6
EEV Sequences

| EEV ID | EEV Sequence |
|---|---|
| EEV1 | Ac-PKKKRKV-K(cyclo[Ff-Nal-GrGrQ])-PEG₁₂-K(N₃)—NH₂ |
| EEV9 | Ac-PKKKRKV-K(cyclo[Ff-Nal-GrGrQ])-miniPEG₂-K(N₃)—NH₂ |
| EEV10 | Ac-PKKKRKV-miniPEG₂-K(cyclo[FGFGRGRQ])-miniPEG₂-K(N₃)—NH₂ |
| EEV5 | Ac-PKKKRKV-miniPEG₂-K(cyclo[FfFGRGRQ])-miniPEG₂-K(N₃)—NH₂ |
| EEV11 | Ac-KKKRK-miniPEG₂-K(cyclo(FGFGRGRQ))-miniPEG₂-K(N₃)—NH₂ |
| EEV12 | Ac-PKKKRKV-miniPEG₂-K(cyclo[FFΦGRGRQ])-PEG₂-K(N₃)—NH₂ |
| EEV13 | Ac-PKKKRKV-miniPEG₂-K(cyclo[βhF-FΦSRSRQ])-PEG₁₂-OH |
| EEV14 | Ac-PKKKRKV-miniPEG₂-K(cyclo[βhF-FΦGRGRQ])-PEG₁₂-OH |
| EEV15 | Ac-PKKKRKV-miniPEG₂-K(cyclo[FGFGRGRQ])-PEG₁₂-K(N₃)—NH₂ |

1-47. (canceled)

48. A method of delivering a therapeutic agent to an eye of a subject, the method comprising administering a therapeutically effective amount of a cargo conjugate to the eye of the subject, the cargo conjugate comprising:
(a) a cargo comprising the therapeutic agent that selectively binds to a target molecule associated with a disease of the eye; and
(b) an ocular delivery construct comprising an exocyclic peptide (EP), cyclic cell penetrating peptide (cCPP) and one or more linkers,
wherein the cargo conjugate has the structure: 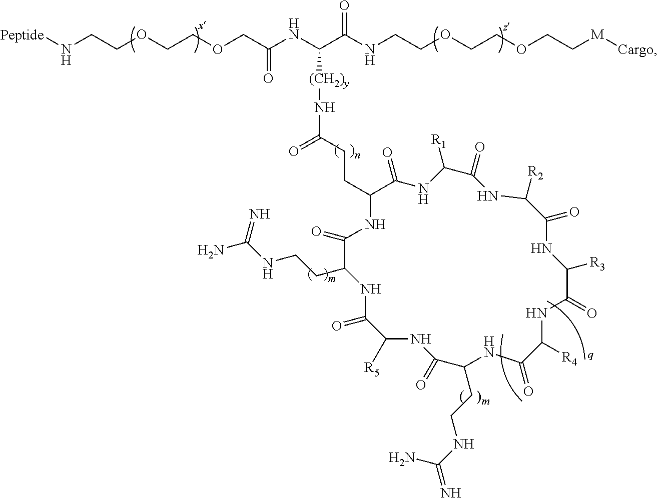
or a protonated form thereof,
wherein:
R₁, R₂, and R₃ are each independently H or an aryl or heteroaryl side chain of an amino acid;
at least two of R₁, R₂, and R₃ are an aryl or heteroaryl side chain of an amino acid;
R₄ and R₆ are independently H or an amino acid side chain;
peptide is an exocyclic peptide (EP) comprising from 2 to 10 amino acids;
M is a bonding group;
each m is independently an integer from 0-3;
n is an integer from 0 to 2;
x′ is an integer from 0 to 20;
y is an integer from 1 to 5;
q is 1 to 4;
z′ is an integer from 1 to 23; and
Cargo is a therapeutic moiety.

49. The method of claim 48, wherein the amino acid residue comprising an aryl or heteroaryl group is phenylalanine or 3-(2-naphthyl)-alanine.

50. The method of claim 48, wherein R₄ and R₆ are, independently, H or a side chain of an amino acid selected from arginine, citrulline, serine or histidine.

51. The method of claim 48, wherein R₄ and R₆ are H.

52. The method of claim 48, wherein R₄ and R₆ are an amino acid side chain of arginine.

53. The method of claim 48, wherein the EP comprises one of the following sequences: PKKKRKV; KR; RR; KKK; KGK; KBK; KBR; KRK; KRR; RKK; RRR; KKKK; KKRK; KRKK; KRRK; RKKR; RRRR; KGKK; KKGK; KKKKK; KKKRK; KBKBK; KKKRKV; PGKKRKV; PKGKRKV; PKKGRKV; PKKKGKV; PKKKRGV; or PKKKRKG.

54. The method of claim 48, wherein the cCPP of the ocular delivery construct has a sequence selected from: FfΦRrRrQ, FGFGRGRQ; GfFGrGrQ, FfΦGRGRQ; FfFGRGRQ; FfΦGrGrQ; FGFGRRRQ; and FGFRRRRQ.

55. The method of claim 48 wherein M comprises 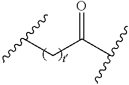
wherein t′ is 0 to 1 or

56. The method of claim 48, wherein M comprises

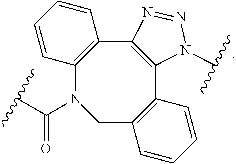 [SMILES: *C(=O)N1Cc2ccccc2-c2c(nnn2*)-c2ccccc21]

57. The method of claim 48, wherein the wherein the ocular delivery construct prior to conjugation to the cargo is selected from:
(a)
Ac-PKKKRKV-PEG_{x′}-K(cyclo[GfFGrGrQ])-PEG_{z′}-OH;
or
Ac-PKKKRKV-PEG_{x′}-K(cyclo[GfFGrGrQ])-PEG_{z′}-K(N₃)—NH₂
(b)
Ac-PKKKRKV-PEG_{x′}-K(cyclo[FfFGRGRQ])-PEG_{z′}-OH;
or
Ac-PKKKRKV-PEG_{x′}-K(cyclo[FfFGRGRQ])-PEG_{z′}-K(N₃)—NH₂
(c)
Ac-PKKKRKV-PEG_{x′}-K(cyclo[Ff-Nal-GrGrQ])-PEG_{z′}-OH;
or
Ac-PKKKRKV-PEG_{x′}-K(cyclo[Ff-Nal-GrGrQ])-PEG_{z′}-K
(N₃)—NH₂
(d)
Ac-PKKKRKV-PEG_{x′}-K(cyclo[FGFGRGRQ])-PEG_{z′}-OH;
or
Ac-PKKKRKV-PEG_{x′}-K(cyclo[FGFGRGRQ])-PEG_{z′}-K(N₃)—NH₂
(e)
Ac-KKKRK-PEG_{x′}-K(cyclo(FGFGRGRQ))-PEG_{z′}-OH;
or
Ac-KKKRK-PEG_{x′}-K(cyclo(FGFGRGRQ))-PEG_{z′}-K(N₃)—NH₂
(f)
Ac-PKKKRKV-PEG_{x′}-K(cyclo[FGFRRRRQ])-PEG_{z′}-OH;
or
Ac-PKKKRKV-PEG_{x′}-K(cyclo[FGFRRRRQ])-PEG_{z′}-K(N₃)—NH₂
(g)
Ac-PKKKRKV-PEG_{x′}-K(cyclo[FFΦGRGRQ])-PEG_{z′}-OH;
or
Ac-PKKKRKV-PEG_{x′}-K(cyclo[FFΦGRGRQ])-PEG_{z′}-K(N3)—NH₂
(h)
Ac-PKKKRKV-PEG_{x′}-K(cyclo[βhF-FΦSRSRQ])-PEG_{z′}-OH;
or
Ac-PKKKRKV-PEG_{x′}-K(cyclo[βhF-FΦSRSRQ])-PEG_{z′}-K
(N₃)—NH₂;
and
(i)
Ac-PKKKRKV-PEG_{x′}-K(cyclo[βhF-FΦGRGRQ])-PEG_{z′}-OH;
or
Ac-PKKKRKV-PEG_{x′}-K(cyclo[βhF-FΦGRGRQ])-PEG_{z′}-K
(N₃)—NH₂,
wherein:
x′ and z′ are, independently, an integer from 0 to 12;
M of the cargo conjugate comprises 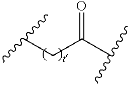
when the ocular delivery construct comprises a terminal carboxylic acid, wherein t′ is 0 or 1; and
M of the cargo conjugate comprises 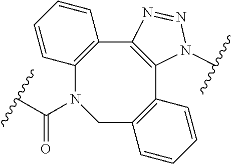 [SMILES: *C(=O)N1Cc2ccccc2-c2c(nnn2*)-c2ccccc21]
when the ocular delivery construct comprises K(N₃).

58. The method of claim 48, wherein the ocular delivery construct prior to conjugation to the cargo is selected from:
(a)
Ac-PKKKRKV-K(cyclo[Ff-Nal-GrGrQ])-PEG₁₂-K(N₃)—NH₂;
Ac-PKKKRKV-K(cyclo[Ff-Nal-GrGrQ])-miniPEG₂-K(N₃)—
NH₂;
Ac-PKKKRKV-PEG₂-K(cyclo[Ff-Nal-GrGrQ])-PEG₁₂-OH;
or
Ac-PKKKRKV-miniPEG₂-K(cyclo[FFΦGRGRQ])-PEG2-K(N₃)—
NH₂;
(b)
Ac-PKKKRKV-PEG₂-K(cyclo[GfFGrGrQ])-PEG₂-K(N₃)—NH₂;
Ac-PKKKRKV-PEG₂-K(cyclo[FfFGRGRQ])-PEG₂-K(N₃)—NH₂;
Ac-PKKKRKV-PEG₂-K(cyclo[FGFGRGRQ])-PEG₁₂-OH;
Ac-PKKKRKV-PEG₂-K(cyclo[FGFRRRRQ])-PEG₁₂-OH;
Ac-PKKKRKV-miniPEG₂-K(cyclo[FGFGRGRQ])-PEG₁₂-K(N₃)—
NH₂;
Ac-PKKKRKV-miniPEG₂-K(cyclo[FGFGRGRQ])-miniPEG₂-K
(N₃)—NH₂;
or
Ac-PKKKRKV-miniPEG₂-K(cyclo[FfFGRGRQ])-miniPEG₂-K
(N₃)—NH₂;
(c)
Ac-KKKRK-miniPEG₂-K(cyclo(FGFGRGRQ))-miniPEG₂-K
(N₃)—NH₂,
wherein
M of the cargo conjugate comprises 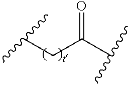
when the ocular delivery construct comprises a terminal carboxylic acid, wherein t′ is 0 or 1; and
M of the cargo conjugate comprises 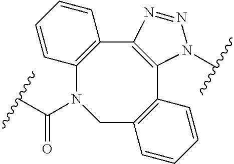 [SMILES: *C(=O)N1Cc2ccccc2-c2c(nnn2*)-c2ccccc21]
when the ocular delivery construct comprises K(N₃).

59. The method of claim 48, wherein the cargo is an antisense oligonucleotide.

60. The method of claim 59, wherein the antisense oligonucleotide is a phosphorodiamidate morpholino oligomer (PMO).

61. The method of claim 48, wherein the disease of the eye is selected from one or more of diabetic retinopathy; glaucoma; retinitis pigmentosa; Usher syndrome; retinal tears or holes; retinal detachment; retinal ischemia; damage associated with laser therapy including photodynamic therapy; surgical light induced iatrogenic retinopathy; drug-induced retinopathies; autosomal dominant optic atrophy; toxic and/or nutritional amblyopias; Leber's hereditary optic neuropathy; atypical retinitis pigmentosa; Bardet-Biedl syndrome; blue-cone monochromacy; cataracts; central areolar choroidal dystrophy; choroideremia; cone dystrophy; rod dystrophy; rod-cone dystrophy; congenital stationary night blindness; cytomegalovirus retinitis; diabetic macular edema; dominant drusen; giant cell arteritis; Goldmann Favre dystrophy; graves' ophthalmopathy; gyrate atrophy; iritis; juvenile retinoschisis; Kearns-Sayre syndrome; Lawrence-Moon syndrome; Leber Congenital Amaurosa; wet macular degeneration; dry macular degeneration; macular dystrophy; ocularhistoplasmosis syndrome; Oguchi disease; oxidative damage; proliferative vitreoretinopathy; refsum disease; retinitis Punctata albescens; retinopathy of prematurity; rod monochromatism; Usher syndrome such as Usher Syndrome type 2A; scleritis; Sjogren-Larsson syndrome; Sorsby fundus dystrophy; Stargardt disease; choroideremia; optic neuropathy; Bietti crystalline dystrophy; Alport syndrome; X-linked retinoschisis; Macula dystrophy; Achromatopsia; congenital stationary night blindness; Best disease; Pattern dystrophy; and Doyne's honeycomb dystrophy.

62. The method of claim 48, wherein administering a cargo conjugate to directly to an eye of the subject comprises topical administration, intravitreal administration, periocular administration, subretinal administration, suprachoroidal administration, or intracameral administration.

63. The method of claim 48, further comprising identifying a subject having an ocular disease.

64. The method of claim 63, wherein the subject has a genetic disease or disorder of the eye.

65. The method of claim 48, wherein administration of the compound modulates expression or activity of a target molecule, the target molecule being a protein or an oligonucleotide.

66. The method of claim 65, wherein administration of the compound downregulates expression or activity of the target molecule.

67. The method of claim 65, wherein administration of the compound upregulates expression or activity of the target molecule.


SEQUENCE LISTING (332 sequences)

SEQ ID NO: 1 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 2 - protein, 8 aa, synthetic construct
FGFGRGRQ

SEQ ID NO: 3 - protein, 8 aa, synthetic construct
  SITE 2: note = D-amino acid
  SITE 5: note = D-amino acid
  SITE 7: note = D-amino acid
GFFGRGRQ

SEQ ID NO: 4 - protein, 8 aa, synthetic construct
  SITE 2: note = D-amino acid
  SITE 3: note = 3-(2-naphthyl)-alanine
FFXGRGRQ

SEQ ID NO: 5 - protein, 8 aa, synthetic construct
  SITE 2: note = D-amino acid
FFFGRGRQ

SEQ ID NO: 6 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 7 - protein, 8 aa, synthetic construct
FGFGRRRQ

SEQ ID NO: 8 - protein, 8 aa, synthetic construct
FGFRRRRQ

SEQ ID NO: 9 - protein, 8 aa, synthetic construct
  SITE 1: note = Beta-homophenylalanine
  SITE 3: note = 3-(2-naphthyl)-alanine
XFXSRSRQ

SEQ ID NO: 10 - protein, 8 aa, synthetic construct
  SITE 1: note = Beta-homophenylalanine
  SITE 3: note = 3-(2-naphthyl)-alanine
XFXGRGRQ

SEQ ID NO: 11 - protein, 7 aa, synthetic construct
PKKKRKV

SEQ ID NO: 12 - protein, 4 aa, synthetic construct
KKKK

SEQ ID NO: 13 - protein, 4 aa, synthetic construct
KKRK

SEQ ID NO: 14 - protein, 4 aa, synthetic construct
KRKK

SEQ ID NO: 15 - protein, 4 aa, synthetic construct
KRRK

SEQ ID NO: 16 - protein, 4 aa, synthetic construct
RKKR

SEQ ID NO: 17 - protein, 4 aa, synthetic construct
RRRR

SEQ ID NO: 18 - protein, 4 aa, synthetic construct
KGKK

SEQ ID NO: 19 - protein, 4 aa, synthetic construct
KKGK

SEQ ID NO: 20 - protein, 5 aa, synthetic construct
KKKKK

SEQ ID NO: 21 - protein, 5 aa, synthetic construct
KKKRK

SEQ ID NO: 22 - protein, 6 aa, synthetic construct
KKKRKV

SEQ ID NO: 23 - protein, 7 aa, synthetic construct
PGKKRKV

SEQ ID NO: 24 - protein, 7 aa, synthetic construct
PKGKRKV

SEQ ID NO: 25 - protein, 7 aa, synthetic construct
PKKGRKV

SEQ ID NO: 26 - protein, 7 aa, synthetic construct
PKKKGKV

SEQ ID NO: 27 - protein, 7 aa, synthetic construct
PKKKRGV

SEQ ID NO: 28 - protein, 7 aa, synthetic construct
PKKKRKG

SEQ ID NO: 29 - protein, 7 aa, synthetic construct
  SITE 2: note = D-amino acid
  SITE 3: note = 3-(2-naphthyl)-alanine
  SITE 5: note = D-amino acid
  SITE 7: note = D-amino acid
FFXRRRR

SEQ ID NO: 30 - protein, 7 aa, synthetic construct
FGFGRGR

SEQ ID NO: 31 - protein, 7 aa, synthetic construct
  SITE 2: note = D-amino acid
  SITE 5: note = D-amino acid
  SITE 7: note = D-amino acid
GFFGRGR

SEQ ID NO: 32 - protein, 7 aa, synthetic construct
  SITE 2: note = D-amino acid
  SITE 3: note = 3-(2-naphthyl)-alanine
FFXGRGR

SEQ ID NO: 33 - protein, 7 aa, synthetic construct
  SITE 2: note = D-amino acid
FFFGRGR

SEQ ID NO: 34 - protein, 7 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGR

SEQ ID NO: 35 - protein, 7 aa, synthetic construct
FGFGRRR

SEQ ID NO: 36 - protein, 7 aa, synthetic construct
FGFRRRR

SEQ ID NO: 37 - protein, 8 aa, synthetic construct
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 38 - protein, 6 aa, synthetic construct
  SITE 2: note = 3-(2-naphthyl)-alanine
FXRRRQ

SEQ ID NO: 39 - protein, 7 aa, synthetic construct
  SITE 6: note = 3-(2-naphthyl)-alanine
FRRRRXQ

SEQ ID NO: 40 - protein, 8 aa, synthetic construct
CRRRRFWQ

SEQ ID NO: 41 - protein, 6 aa, synthetic construct
  SITE 2: note = 3-(2-naphthyl)-alanine
FXRRRC

SEQ ID NO: 42 - protein, 7 aa, synthetic construct
  SITE 1: note = D-amino acid
  SITE 5: note = 3-(2-naphthyl)-alanine
RRFRXRQ

SEQ ID NO: 43 - protein, 8 aa, synthetic construct
  SITE 2: note = 3-(2-naphthyl)-alanine
FXRRRRQK

SEQ ID NO: 44 - protein, 6 aa, synthetic construct
  SITE 2: note = 3-(2-naphthyl)-alanine
FXRRRU

SEQ ID NO: 45 - protein, 7 aa, synthetic construct
  SITE 3: note = 3-(2-naphthyl)-alanine
RRXFRRQ

SEQ ID NO: 46 - protein, 8 aa, synthetic construct
  SITE 2: note = 3-(2-naphthyl)-alanine
FXRRRRQC

SEQ ID NO: 47 - protein, 6 aa, synthetic construct
  SITE 4: note = 3-(2-naphthyl)-alanine
RRRXFQ

SEQ ID NO: 48 - protein, 8 aa, synthetic construct
  SITE 1: note = D-amino acid
  SITE 2: note = 3-(2-naphthyl)-alanine
  SITE 4: note = D-amino acid
  SITE 6: note = D-amino acid
FXRRRRRQ

SEQ ID NO: 49 - protein, 6 aa, synthetic construct
  SITE 5: note = 3-(2-naphthyl)-alanine
RRRRXF

SEQ ID NO: 50 - protein, 8 aa, synthetic construct
  SITE 2: note = 3-(2-naphthyl)-alanine
FXRRRRRQ

SEQ ID NO: 51 - protein, 6 aa, synthetic construct
  SITE 2: note = 3-(2-naphthyl)-alanine
FXRRRR

SEQ ID NO: 52 - protein, 8 aa, synthetic construct
  SITE 3: note = 3-(2-naphthyl)-alanine
FFXRRRRQ

SEQ ID NO: 53 - protein, 9 aa, synthetic construct
  SITE 5: note = 3-(2-naphthyl)-alanine
  SITE 8: note = Norleucine
RRRRXFDXC

SEQ ID NO: 54 - protein, 7 aa, synthetic construct
  SITE 2: note = D-3-(2-naphthyl)-alanine
  SITE 3: note = D-amino acid
  SITE 5: note = D-amino acid
  SITE 7: note = D-amino acid
FXRRRRQ

SEQ ID NO: 55 - protein, 8 aa, synthetic construct
  SITE 6: note = 3-(2-naphthyl)-alanine
RFRFRXRQ

SEQ ID NO: 56 - protein, 5 aa, synthetic construct
  SITE 2: note = 3-(2-naphthyl)-alanine
FXRRR

SEQ ID NO: 57 - protein, 7 aa, synthetic construct
  SITE 2: note = D-3-(2-naphthyl)-alanine
  SITE 3: note = D-amino acid
  SITE 5: note = D-amino acid
FXRRRRQ

SEQ ID NO: 58 - protein, 8 aa, synthetic construct
URRRRFWQ

SEQ ID NO: 59 - protein, 5 aa, synthetic construct
FWRRR

SEQ ID NO: 60 - protein, 7 aa, synthetic construct
  SITE 2: note = 3-(2-naphthyl)-alanine
FXRRRRQ

SEQ ID NO: 61 - protein, 7 aa, synthetic construct
  SITE 5: note = 3-(2-naphthyl)-alanine
RRFRXRQ

SEQ ID NO: 62 - protein, 5 aa, synthetic construct
  SITE 4: note = 3-(2-naphthyl)-alanine
RRRXF

SEQ ID NO: 63 - protein, 7 aa, synthetic construct
  SITE 1: note = D-amino acid
  SITE 2: note = 3-(2-naphthyl)-alanine
  SITE 4: note = D-amino acid
  SITE 6: note = D-amino acid
FXRRRRQ

SEQ ID NO: 64 - protein, 5 aa, synthetic construct
RRRWF

SEQ ID NO: 65 - protein, 7 aa, synthetic construct
FGFGHGH

SEQ ID NO: 66 - protein, 7 aa, synthetic construct
FGFSHSH

SEQ ID NO: 67 - protein, 8 aa, synthetic construct
FGFGHGHQ

SEQ ID NO: 68 - protein, 8 aa, synthetic construct
FGFSHSHQ

SEQ ID NO: 69 - protein, 7 aa, synthetic construct
  SITE 1: note = Beta-homophenylalanine
  SITE 3: note = 3-(2-naphthyl)-alanine
XFXSRSR

SEQ ID NO: 70 - protein, 7 aa, synthetic construct
  SITE 1: note = Beta-homophenylalanine
  SITE 3: note = 3-(2-naphthyl)-alanine
XFXGRGR

SEQ ID NO: 71 - protein, 7 aa, synthetic construct
  SITE 1: note = Beta-homophenylalanine
  SITE 2: note = D-amino acid
  SITE 3: note = 3-(2-naphthyl)-alanine
  SITE 5: note = D-amino acid
  SITE 7: note = D-amino acid
XFXGRGR

SEQ ID NO: 72 - protein, 7 aa, synthetic construct
  SITE 1: note = Beta-homophenylalanine
  SITE 2: note = D-amino acid
  SITE 3: note = 3-(2-naphthyl)-alanine
XFXSRSR

SEQ ID NO: 73 - protein, 7 aa, synthetic construct
  SITE 2: note = D-amino acid
  SITE 3: note = 3-(2-naphthyl)-alanine
  SITE 5: note = D-amino acid
  SITE 7: note = D-amino acid
FFXSRSR

SEQ ID NO: 74 - protein, 8 aa, synthetic construct
  SITE 1: note = Beta-homophenylalanine
  SITE 2: note = D-amino acid
  SITE 3: note = 3-(2-naphthyl)-alanine
  SITE 5: note = D-amino acid
  SITE 7: note = D-amino acid
XFXGRGRQ

SEQ ID NO: 75 - protein, 8 aa, synthetic construct
  SITE 1: note = Beta-homophenylalanine
  SITE 2: note = D-amino acid
  SITE 3: note = 3-(2-naphthyl)-alanine
XFXSRSRQ

SEQ ID NO: 76 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXSRSRQ

SEQ ID NO: 77 - protein, 7 aa, synthetic construct
  SITE 2: note = D-amino acid
FFFSRSR

SEQ ID NO: 78 - protein, 7 aa, synthetic construct
FGFSRSR

SEQ ID NO: 79 - protein, 8 aa, synthetic construct
  SITE 2: note = D-amino acid
FFFSRSRQ

SEQ ID NO: 80 - protein, 8 aa, synthetic construct
FGFSRSRQ

SEQ ID NO: 81 - protein, 8 aa, synthetic construct
  SITE 1: note = Beta-homophenylalanine
  SITE 2: note = D-amino acid
  SITE 3: note = 3-(2-naphthyl)-alanine
  SITE 5: note = D-amino acid
  SITE 7: note = D-amino acid
XFXSRSRQ

SEQ ID NO: 82 - protein, 8 aa, synthetic construct
  SITE 1: note = Beta-homophenylalanine
  SITE 2: note = D-amino acid
  SITE 3: note = 3-(2-naphthyl)-alanine
  SITE 5: note = D-amino acid
  SITE 7: note = D-amino acid
XFXHRHRQ

SEQ ID NO: 83 - protein, 8 aa, synthetic construct
  SITE 1: note = Beta-homophenylalanine
  SITE 3: note = 3-(2-naphthyl)-alanine
XFXHRHRQ

SEQ ID NO: 84 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXHRHRQ

SEQ ID NO: 85 - protein, 4 aa, synthetic construct
  VARIANT 1..4: note = May have D or L stereochemistry
HHHH

SEQ ID NO: 86 - protein, 4 aa, synthetic construct
  VARIANT 1..4: note = May have D or L stereochemistry
KHKK

SEQ ID NO: 87 - protein, 4 aa, synthetic construct
  VARIANT 1..4: note = May have D or L stereochemistry
KKHK

SEQ ID NO: 88 - protein, 4 aa, synthetic construct
  VARIANT 1..4: note = May have D or L stereochemistry
KKKH

SEQ ID NO: 89 - protein, 4 aa, synthetic construct
  VARIANT 1..4: note = May have D or L stereochemistry
KHKH

SEQ ID NO: 90 - protein, 4 aa, synthetic construct
  VARIANT 1..4: note = May have D or L stereochemistry
HKHK

SEQ ID NO: 91 - protein, 4 aa, synthetic construct
  VARIANT 1..4: note = May have D or L stereochemistry
KKKK

SEQ ID NO: 92 - protein, 4 aa, synthetic construct
  VARIANT 1..4: note = May have D or L stereochemistry
KKRK

SEQ ID NO: 93 - protein, 4 aa, synthetic construct
  VARIANT 1..4: note = May have D or L stereochemistry
KRKK

SEQ ID NO: 94 - protein, 4 aa, synthetic construct
  VARIANT 1..4: note = May have D or L stereochemistry
KRRK

SEQ ID NO: 95 - protein, 4 aa, synthetic construct
  VARIANT 1..4: note = May have D or L stereochemistry
RKKR

SEQ ID NO: 96 - protein, 4 aa, synthetic construct
  VARIANT 1..4: note = May have D or L stereochemistry
RRRR

SEQ ID NO: 97 - protein, 4 aa, synthetic construct
  VARIANT 1..4: note = May have D or L stereochemistry
KGKK

SEQ ID NO: 98 - protein, 4 aa, synthetic construct
  VARIANT 1..4: note = May have D or L stereochemistry
KKGK

SEQ ID NO: 99 - protein, 5 aa, synthetic construct
  VARIANT 1..5: note = May have D or L stereochemistry
RRRRR

SEQ ID NO: 100 - protein, 5 aa, synthetic construct
  VARIANT 1..5: note = May have D or L stereochemistry
KKKKK

SEQ ID NO: 101 - protein, 5 aa, synthetic construct
  VARIANT 1..5: note = May have D or L stereochemistry
KKKRK

SEQ ID NO: 102 - protein, 5 aa, synthetic construct
  VARIANT 1..5: note = May have D or L stereochemistry
RKKKK

SEQ ID NO: 103 - protein, 5 aa, synthetic construct
  VARIANT 1..5: note = May have D or L stereochemistry
KRKKK

SEQ ID NO: 104 - protein, 5 aa, synthetic construct
  VARIANT 1..5: note = May have D or L stereochemistry
KKRKK

SEQ ID NO: 105 - protein, 5 aa, synthetic construct
  VARIANT 1..5: note = May have D or L stereochemistry
KKKKR

SEQ ID NO: 106 - protein, 6 aa, synthetic construct
  VARIANT 1..6: note = May have D or L stereochemistry
RKKKKG

SEQ ID NO: 107 - protein, 6 aa, synthetic construct
  VARIANT 1..6: note = May have D or L stereochemistry
KRKKKG

SEQ ID NO: 108 - protein, 6 aa, synthetic construct
  VARIANT 1..6: note = May have D or L stereochemistry
KKRKKG

SEQ ID NO: 109 - protein, 6 aa, synthetic construct
  VARIANT 1..6: note = May have D or L stereochemistry
KKKKRG

SEQ ID NO: 110 - protein, 6 aa, synthetic construct
  SITE 6: note = Beta-alanine
  VARIANT 1..6: note = May have D or L stereochemistry
RKKKKX

SEQ ID NO: 111 - protein, 6 aa, synthetic construct
  SITE 6: note = Beta-alanine
  VARIANT 1..6: note = May have D or L stereochemistry
KRKKKX

SEQ ID NO: 112 - protein, 6 aa, synthetic construct
  SITE 6: note = Beta-alanine
  VARIANT 1..6: note = May have D or L stereochemistry
KKRKKX

SEQ ID NO: 113 - protein, 6 aa, synthetic construct
  SITE 6: note = Beta-alanine
  VARIANT 1..6: note = May have D or L stereochemistry
KKKKRX

SEQ ID NO: 114 - protein, 6 aa, synthetic construct
  VARIANT 1..6: note = May have D or L stereochemistry
KKKRKV

SEQ ID NO: 115 - protein, 6 aa, synthetic construct
  VARIANT 1..6: note = May have D or L stereochemistry
RRRRRR

SEQ ID NO: 116 - protein, 6 aa, synthetic construct
  VARIANT 1..6: note = May have D or L stereochemistry
HHHHHH

SEQ ID NO: 117 - protein, 6 aa, synthetic construct
  VARIANT 1..6: note = May have D or L stereochemistry
RHRHRH

SEQ ID NO: 118 - protein, 6 aa, synthetic construct
  VARIANT 1..6: note = May have D or L stereochemistry
HRHRHR

SEQ ID NO: 119 - protein, 6 aa, synthetic construct
  VARIANT 1..6: note = May have D or L stereochemistry
KRKRKR

SEQ ID NO: 120 - protein, 6 aa, synthetic construct
  VARIANT 1..6: note = May have D or L stereochemistry
RKRKRK

SEQ ID NO: 121 - protein, 7 aa, synthetic construct
  VARIANT 1..7: note = May have D or L stereochemistry
PKKKRKV

SEQ ID NO: 122 - protein, 7 aa, synthetic construct
  VARIANT 1..7: note = May have D or L stereochemistry
PGKKRKV

SEQ ID NO: 123 - protein, 7 aa, synthetic construct
  VARIANT 1..7: note = May have D or L stereochemistry
PKGKRKV

SEQ ID NO: 124 - protein, 7 aa, synthetic construct
  VARIANT 1..7: note = May have D or L stereochemistry
PKKGRKV

SEQ ID NO: 125 - protein, 7 aa, synthetic construct
  VARIANT 1..7: note = May have D or L stereochemistry
PKKKGKV

SEQ ID NO: 126 - protein, 7 aa, synthetic construct
  VARIANT 1..7: note = May have D or L stereochemistry
PKKKRGV

SEQ ID NO: 127 - protein, 7 aa, synthetic construct
  VARIANT 1..7: note = May have D or L stereochemistry
PKKKRKG

SEQ ID NO: 128 - protein, 19 aa, synthetic construct
NLSKRPAAIKKAGQAKKKK

SEQ ID NO: 129 - protein, 9 aa, synthetic construct
PAAKRVKLD

SEQ ID NO: 130 - protein, 11 aa, synthetic construct
RQRRNELKRSF

SEQ ID NO: 131 - protein, 27 aa, synthetic construct
RMRKFKNKGKDTAELRRRRVEVSVELR

SEQ ID NO: 132 - protein, 14 aa, synthetic construct
KAKKDEQILKRRNV

SEQ ID NO: 133 - protein, 8 aa, synthetic construct
VSRKRPRP

SEQ ID NO: 134 - protein, 8 aa, synthetic construct
PPKKARED

SEQ ID NO: 135 - protein, 8 aa, synthetic construct
PQPKKKPL

SEQ ID NO: 136 - protein, 12 aa, synthetic construct
SALIKKKKKMAP

SEQ ID NO: 137 - protein, 5 aa, synthetic construct
DRLRR

SEQ ID NO: 138 - protein, 7 aa, synthetic construct
PKQKKRK

SEQ ID NO: 139 - protein, 10 aa, synthetic construct
RKLKKKIKKL

SEQ ID NO: 140 - protein, 10 aa, synthetic construct
REKKKFLKRR

SEQ ID NO: 141 - protein, 20 aa, synthetic construct
KRKGDEVDGVDEVAKKKSKK

SEQ ID NO: 142 - protein, 17 aa, synthetic construct
RKCLQAGMNLEARKTKK

SEQ ID NO: 143 - protein, 7 aa, synthetic construct
  SITE 1: note = Acetylated residue
PKKKRKV

SEQ ID NO: 144 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 145 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 146 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 147 - protein, 8 aa, synthetic construct
  VARIANT 1: note = Residue attached to another sequence via a Lysine 0-12x polyethylene glycol linker
  SITE 2: note = D-amino acid
  SITE 5: note = D-amino acid
  SITE 7: note = D-amino acid
GFFGRGRQ

SEQ ID NO: 148 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 149 - protein, 8 aa, synthetic construct
  VARIANT 1: note = Residue attached to another sequence via a Lysine 0-12x polyethylene glycol linker
  SITE 2: note = D-amino acid
  SITE 5: note = D-amino acid
  SITE 7: note = D-amino acid
GFFGRGRQ

SEQ ID NO: 150 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 151 - protein, 8 aa, synthetic construct
  VARIANT 1: note = Residue attached to another sequence via a Lysine 0-12x polyethylene glycol linker
  SITE 2: note = D-amino acid
FFFGRGRQ

SEQ ID NO: 152 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 153 - protein, 8 aa, synthetic construct
  VARIANT 1: note = Residue attached to another sequence via a Lysine 0-12x polyethylene glycol linker
  SITE 2: note = D-amino acid
FFFGRGRQ

SEQ ID NO: 154 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 155 - protein, 8 aa, synthetic construct
  VARIANT 1: note = Residue attached to another sequence via a Lysine 0-12x polyethylene glycol linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 156 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 157 - protein, 8 aa, synthetic construct
  VARIANT 1: note = Residue attached to another sequence via a Lysine 0-12x polyethylene glycol linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 158 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 159 - protein, 8 aa, synthetic construct
  VARIANT 1: note = Residue attached to another sequence via a Lysine 0-12x polyethylene glycol linker
FGFGRGRQ

SEQ ID NO: 160 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 161 - protein, 8 aa, synthetic construct
  VARIANT 1: note = Residue attached to another sequence via a Lysine 0-12x polyethylene glycol linker
FGFGRGRQ

SEQ ID NO: 162 - protein, 5 aa, synthetic construct
  SITE 1: note = Acetylated residue
  VARIANT 5: note = Residue attached to another sequence via a Lysine 0-12x polyethylene glycol linker
KKKRK

SEQ ID NO: 163 - protein, 8 aa, synthetic construct
  VARIANT 1: note = Residue attached to another sequence via a Lysine 0-12x polyethylene glycol linker
FGFGRGRQ

SEQ ID NO: 164 - protein, 5 aa, synthetic construct
  SITE 1: note = Acetylated residue
  VARIANT 5: note = Residue attached to another sequence via a Lysine 0-12x polyethylene glycol linker
KKKRK

SEQ ID NO: 165 - protein, 8 aa, synthetic construct
  VARIANT 1: note = Residue attached to another sequence via a Lysine 0-12x polyethylene glycol linker
FGFGRGRQ

SEQ ID NO: 166 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 167 - protein, 8 aa, synthetic construct
  VARIANT 1: note = Residue attached to another sequence via a Lysine 0-12x polyethylene glycol linker
FGFRRRRQ

SEQ ID NO: 168 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 169 - protein, 8 aa, synthetic construct
  VARIANT 1: note = Residue attached to another sequence via a Lysine 0-12x polyethylene glycol linker
FGFRRRRQ

SEQ ID NO: 170 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 171 - protein, 8 aa, synthetic construct
  VARIANT 1: note = Residue attached to another sequence via a Lysine 0-12x polyethylene glycol linker
  SITE 3: note = 3-(2-naphthyl)-alanine
FFXGRGRQ

SEQ ID NO: 172 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 173 - protein, 8 aa, synthetic construct
  VARIANT 1: note = Residue attached to another sequence via a Lysine 0-12x polyethylene glycol linker
  SITE 3: note = 3-(2-naphthyl)-alanine
FFXGRGRQ

SEQ ID NO: 174 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 175 - protein, 8 aa, synthetic construct
  VARIANT 1: note = Beta-homophenylalanine attached to another sequence via a Lysine 0-12x polyethylene glycol linker
  SITE 3: note = 3-(2-naphthyl)-alanine
XFXSRSRQ

SEQ ID NO: 176 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 177 - protein, 8 aa, synthetic construct
  VARIANT 1: note = Beta-homophenylalanine attached to another sequence via a Lysine 0-12x polyethylene glycol linker
  SITE 3: note = 3-(2-naphthyl)-alanine
XFXSRSRQ

SEQ ID NO: 178 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 179 - protein, 8 aa, synthetic construct
  VARIANT 1: note = Beta-homophenylalanine attached to another sequence via a Lysine 0-12x polyethylene glycol linker
  SITE 3: note = 3-(2-naphthyl)-alanine
XFXGRGRQ

SEQ ID NO: 180 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 181 - protein, 8 aa, synthetic construct
  VARIANT 1: note = Beta-homophenylalanine attached to another sequence via a Lysine 0-12x polyethylene glycol linker
  SITE 3: note = 3-(2-naphthyl)-alanine
XFXGRGRQ

SEQ ID NO: 182 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 183 - protein, 8 aa, synthetic construct
  VARIANT 1: note = Beta-homophenylalanine attached to another sequence via a Lysine 0-12x polyethylene glycol linker
  SITE 2: note = D-amino acid
  SITE 3: note = 3-(2-naphthyl)-alanine
  SITE 5: note = D-amino acid
  SITE 7: note = D-amino acid
XFXGRGRQ

SEQ ID NO: 184 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 185 - protein, 8 aa, synthetic construct
  VARIANT 1: note = Beta-homophenylalanine attached to another sequence via a Lysine 0-12x polyethylene glycol linker
  SITE 2: note = D-amino acid
  SITE 3: note = 3-(2-naphthyl)-alanine
  SITE 5: note = D-amino acid
  SITE 7: note = D-amino acid
XFXGRGRQ

SEQ ID NO: 186 - protein, 7 aa, synthetic construct
  SITE 1: note = Acetylated residue
  VARIANT 7: note = Residue attached to another sequence via a Lysine 0-12x polyethylene glycol linker
PKKKRKV

SEQ ID NO: 187 - protein, 8 aa, synthetic construct
  VARIANT 1: note = Beta-homophenylalanine attached to another sequence via a Lysine 0-12x polyethylene glycol linker
  SITE 2: note = D-amino acid
  SITE 3: note = 3-(2-naphthyl)-alanine
XFXSRSRQ

SEQ ID NO: 188 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 189 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 190 - protein, 8 aa, synthetic construct
  SITE 1: note = Acetylated residue
  SITE 8: note = Residue attached to another sequence via the side chain
PKKKRKVK

SEQ ID NO: 191 - protein, 8 aa, synthetic construct
  SITE 1: note = Residue attached to another sequence via the side chain
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 192 - protein, 8 aa, synthetic construct
  SITE 1: note = Acetylated residue
  SITE 8: note = Residue attached to another sequence via the side chain
PKKKRKVK

SEQ ID NO: 193 - protein, 8 aa, synthetic construct
  SITE 1: note = Residue attached to another sequence via the side chain
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 194 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 195 - protein, 8 aa, synthetic construct
  SITE 1: note = Residue attached to another sequence via a Lysine 2x polyethylene glycol linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 196 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 197 - protein, 8 aa, synthetic construct
  SITE 1: note = Residue attached to another sequence via a Lysine 2-[2-[2-aminoethoxy]ethoxy]acetic acid linker
  SITE 3: note = 3-(2-naphthyl)-alanine
FFXGRGRQ

SEQ ID NO: 198 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 199 - protein, 8 aa, synthetic construct
  SITE 1: note = Residue attached to another sequence via a Lysine 2x polyethylene glycol linker
  SITE 2: note = D-amino acid
  SITE 5: note = D-amino acid
  SITE 7: note = D-amino acid
GFFGRGRQ

SEQ ID NO: 200 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 201 - protein, 8 aa, synthetic construct
  SITE 1: note = Residue attached to another sequence via a Lysine 2x polyethylene glycol linker
  SITE 2: note = D-amino acid
FFFGRGRQ

SEQ ID NO: 202 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 203 - protein, 8 aa, synthetic construct
  SITE 1: note = Residue attached to another sequence via a Lysine 2x polyethylene glycol linker
FGFGRGRQ

SEQ ID NO: 204 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 205 - protein, 8 aa, synthetic construct
  SITE 1: note = Residue attached to another sequence via a Lysine 2x polyethylene glycol linker
FGFRRRRQ

SEQ ID NO: 206 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 207 - protein, 8 aa, synthetic construct
  SITE 1: note = Residue attached to another sequence via a Lysine 2-[2-[2-aminoethoxy]ethoxy]acetic acid linker
FGFGRGRQ

SEQ ID NO: 208 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 209 - protein, 8 aa, synthetic construct
  SITE 1: note = Residue attached to another sequence via a Lysine 2-[2-[2-aminoethoxy]ethoxy]acetic acid linker
FGFGRGRQ

SEQ ID NO: 210 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 211 - protein, 8 aa, synthetic construct
  SITE 1: note = Residue attached to another sequence via a Lysine 2-[2-[2-aminoethoxy]ethoxy]acetic acid linker
  SITE 2: note = D-amino acid
FFFGRGRQ

SEQ ID NO: 212 - protein, 5 aa, synthetic construct
  SITE 1: note = Acetylated residue
  SITE 5: note = Residue attached to another sequence via a Lysine 2-[2-[2-aminoethoxy]ethoxy]acetic acid linker
KKKRK

SEQ ID NO: 213 - protein, 8 aa, synthetic construct
  SITE 1: note = Residue attached to another sequence via a Lysine 2-[2-[2-aminoethoxy]ethoxy]acetic acid linker
FGFGRGRQ

SEQ ID NO: 214 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 215 - protein, 8 aa, synthetic construct
  SITE 1: note = Beta-homophenylalanine attached to another sequence via a Lysine 2-[2-[2-aminoethoxy]ethoxy]acetic acid linker
  SITE 3: note = 3-(2-naphthyl)-alanine
XFXSRSRQ

SEQ ID NO: 216 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 217 - protein, 8 aa, synthetic construct
  SITE 1: note = Beta-homophenylalanine attached to another sequence via a Lysine 2-[2-[2-aminoethoxy]ethoxy]acetic acid linker
  SITE 3: note = 3-(2-naphthyl)-alanine
XFXGRGRQ

SEQ ID NO: 218 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 219 - protein, 8 aa, synthetic construct
  SITE 1: note = Beta-homophenylalanine attached to another sequence via a Lysine 2-[2-[2-aminoethoxy]ethoxy]acetic acid linker
  SITE 2: note = D-amino acid
  SITE 3: note = 3-(2-naphthyl)-alanine
  SITE 5: note = D-amino acid
  SITE 7: note = D-amino acid
XFXGRGRQ

SEQ ID NO: 220 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 221 - protein, 8 aa, synthetic construct
  SITE 1: note = Beta-homophenylalanine attached to another sequence via a Lysine 2-[2-[2-aminoethoxy]ethoxy]acetic acid linker
  SITE 2: note = D-amino acid
  SITE 3: note = 3-(2-naphthyl)-alanine
XFXSRSRQ

SEQ ID NO: 222 - protein, 8 aa, synthetic construct
  SITE 1: note = Acetylated residue
  SITE 8: note = Residue attached to another sequence via the side chain
PKKKRKVK

SEQ ID NO: 223 - protein, 8 aa, synthetic construct
  SITE 1: note = Residue attached to another sequence via the side chain
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 224 - protein, 5 aa, synthetic construct
  SITE 1: note = Acetylated residue
  VARIANT 5: note = Residue attached to another sequence via a Lysine 0-12x polyethylene glycol linker
KKKRK

SEQ ID NO: 225 - protein, 8 aa, synthetic construct
  VARIANT 1: note = Residue attached to another sequence via a Lysine 0-12x polyethylene glycol linker
FGFGRGRQ

SEQ ID NO: 226 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 227 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 228 - protein, 8 aa, synthetic construct
  SITE 1: note = Residue attached to another sequence via a Lysine 2x polyethylene glycol linker
  SITE 2: note = D-amino acid
  SITE 5: note = D-amino acid
  SITE 7: note = D-amino acid
GFFGRGRQ

SEQ ID NO: 229 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 230 - protein, 8 aa, synthetic construct
  SITE 1: note = Residue attached to another sequence via a Lysine 2x polyethylene glycol linker
  SITE 2: note = D-amino acid
FFFGRGRQ

SEQ ID NO: 231 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 232 - protein, 8 aa, synthetic construct
  SITE 1: note = Residue attached to another sequence via a Lysine 2x polyethylene glycol linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 233 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 234 - protein, 8 aa, synthetic construct
  SITE 1: note = Residue attached to another sequence via a Lysine 2x polyethylene glycol linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 235 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 236 - protein, 8 aa, synthetic construct
  SITE 1: note = Residue attached to another sequence via a Lysine 2x polyethylene glycol linker
FGFGRGRQ

SEQ ID NO: 237 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 238 - protein, 8 aa, synthetic construct
  SITE 1: note = Residue attached to another sequence via a Lysine 2x polyethylene glycol linker
FGFGRGRQ

SEQ ID NO: 239 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 240 - protein, 8 aa, synthetic construct
  SITE 1: note = Residue attached to another sequence via a Lysine 2x polyethylene glycol linker
FGFRRRRQ

SEQ ID NO: 241 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 242 - protein, 8 aa, synthetic construct
  SITE 1: note = Residue attached to another sequence via a Lysine 2x polyethylene glycol linker
FGFRRRRQ

SEQ ID NO: 243 - protein, 8 aa, synthetic construct
  SITE 1: note = Acetylated residue
  SITE 8: note = Residue attached to another sequence via the side chain
PKKKRKVK

SEQ ID NO: 244 - protein, 8 aa, synthetic construct
  SITE 1: note = Residue attached to another sequence via the side chain
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 245 - protein, 8 aa, synthetic construct
  SITE 1: note = Acetylated residue
  SITE 8: note = Residue attached to another sequence via the side chain
PKKKRKVK

SEQ ID NO: 246 - protein, 8 aa, synthetic construct
  SITE 1: note = Residue attached to another sequence via the side chain
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 247 - protein, 8 aa, synthetic construct
  SITE 1: note = Acetylated residue
  SITE 8: note = Residue attached to another sequence via the side chain
PKKKRKVK

SEQ ID NO: 248 - protein, 7 aa, synthetic construct
  SITE 1: note = D-amino acid attached to another sequence via the side chain
  SITE 2: note = 3-(2-naphthyl)-alanine
  SITE 4: note = D-amino acid
  SITE 6: note = D-amino acid
FXGRGRQ

SEQ ID NO: 249 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 250 - protein, 8 aa, synthetic construct
  SITE 1: note = Residue attached to another sequence via a Lysine 2x polyethylene glycol linker
FGFGRGRQ

SEQ ID NO: 251 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 252 - protein, 8 aa, synthetic construct
  SITE 1: note = Residue attached to another sequence via a Lysine 2x polyethylene glycol linker
FGFGRGRQ

SEQ ID NO: 253 - protein, 5 aa, synthetic construct
  SITE 1: note = Acetylated residue
  SITE 5: note = Residue attached to another sequence via a Lysine 2x polyethylene glycol linker
KKKRK

SEQ ID NO: 254 - protein, 8 aa, synthetic construct
  SITE 1: note = Residue attached to another sequence via a Lysine 2x polyethylene glycol linker
FGFGRGRQ

SEQ ID NO: 255 - protein, 5 aa, synthetic construct
  SITE 1: note = Acetylated residue
  SITE 5: note = Residue attached to another sequence via a Lysine 2x polyethylene glycol linker
KKKRK

SEQ ID NO: 256 - protein, 8 aa, synthetic construct
  SITE 1: note = Residue attached to another sequence via a Lysine 2x polyethylene glycol linker
FGFGRGRQ

SEQ ID NO: 257 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 258 - protein, 8 aa, synthetic construct
  SITE 1: note = Residue attached to another sequence via a Lysine 2x polyethylene glycol linker
  SITE 3: note = 3-(2-naphthyl)-alanine
FFXGRGRQ

SEQ ID NO: 259 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 260 - protein, 8 aa, synthetic construct
  SITE 1: note = Residue attached to another sequence via a Lysine 2x polyethylene glycol linker
  SITE 3: note = 3-(2-naphthyl)-alanine
FFXGRGRQ

SEQ ID NO: 261 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 262 - protein, 8 aa, synthetic construct
  SITE 1: note = Beta-homophenylalanine attached to another sequence via a Lysine 2x polyethylene glycol linker
  SITE 3: note = 3-(2-naphthyl)-alanine
XFXSRSRQ

SEQ ID NO: 263 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 264 - protein, 8 aa, synthetic construct
  SITE 1: note = Beta-homophenylalanine attached to another sequence via a Lysine 2x polyethylene glycol linker
  SITE 3: note = 3-(2-naphthyl)-alanine
XFXSRSRQ

SEQ ID NO: 265 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 266 - protein, 8 aa, synthetic construct
  SITE 1: note = Beta-homophenylalanine attached to another sequence via a Lysine 2x polyethylene glycol linker
  SITE 3: note = 3-(2-naphthyl)-alanine
XFXGRGRQ

SEQ ID NO: 267 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 268 - protein, 8 aa, synthetic construct
  SITE 1: note = Beta-homophenylalanine attached to another sequence via a Lysine 2x polyethylene glycol linker
  SITE 3: note = 3-(2-naphthyl)-alanine
XFXGRGRQ

SEQ ID NO: 269 - protein, 8 aa, synthetic construct
  SITE 1: note = Acetylated residue
  SITE 8: note = Residue attached to another sequence via the side chain
PKKKRKVK

SEQ ID NO: 270 - protein, 8 aa, synthetic construct
  SITE 1: note = Residue attached to another sequence via the side chain
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 271 - protein, 5 aa, synthetic construct
  SITE 1: note = Acetylated residue
  VARIANT 5: note = Residue attached to another sequence via a Lysine 0-12x polyethylene glycol linker
KKKRK

SEQ ID NO: 272 - protein, 8 aa, synthetic construct
  VARIANT 1: note = Residue attached to another sequence via a Lysine 0-12x polyethylene glycol linker
FGFGRGRQ

SEQ ID NO: 273 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 274 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 275 - protein, 8 aa, synthetic construct
  SITE 1: note = Residue attached to another sequence via a Lysine 2x polyethylene glycol linker
  SITE 2: note = D-amino acid
  SITE 5: note = D-amino acid
  SITE 7: note = D-amino acid
GFFGRGRQ

SEQ ID NO: 276 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 277 - protein, 8 aa, synthetic construct
  SITE 1: note = Residue attached to another sequence via a Lysine 2x polyethylene glycol linker
  SITE 2: note = D-amino acid
  SITE 5: note = D-amino acid
  SITE 7: note = D-amino acid
GFFGRGRQ

SEQ ID NO: 278 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 279 - protein, 8 aa, synthetic construct
  SITE 1: note = Residue attached to another sequence via a Lysine 2x polyethylene glycol linker
  SITE 2: note = D-amino acid
FFFGRGRQ

SEQ ID NO: 280 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 281 - protein, 8 aa, synthetic construct
  SITE 1: note = Residue attached to another sequence via a Lysine 2x polyethylene glycol linker
  SITE 2: note = D-amino acid
FFFGRGRQ

SEQ ID NO: 282 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 283 - protein, 8 aa, synthetic construct
  SITE 1: note = Residue attached to another sequence via a Lysine 2x polyethylene glycol linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 284 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 285 - protein, 8 aa, synthetic construct
  SITE 1: note = Residue attached to another sequence via a Lysine 2x polyethylene glycol linker
FGFGRGRQ

SEQ ID NO: 286 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 287 - protein, 8 aa, synthetic construct
  SITE 1: note = Residue attached to another sequence via a Lysine 2x polyethylene glycol linker
FGFRRRRQ

SEQ ID NO: 288 - protein, 8 aa, synthetic construct
  SITE 1: note = Acetylated residue
  SITE 8: note = Residue attached to another sequence via the side chain
PKKKRKVK

SEQ ID NO: 289 - protein, 8 aa, synthetic construct
  SITE 1: note = Residue attached to another sequence via the side chain
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 290 - protein, 8 aa, synthetic construct
  SITE 1: note = Acetylated residue
  SITE 8: note = Residue attached to another sequence via the side chain
PKKKRKVK

SEQ ID NO: 291 - protein, 8 aa, synthetic construct
  SITE 1: note = Residue attached to another sequence via the side chain
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 292 - protein, 5 aa, synthetic construct
  SITE 1: note = Acetylated residue
  SITE 5: note = Residue attached to another sequence via a Lysine 2x polyethylene glycol linker
KKKRK

SEQ ID NO: 293 - protein, 8 aa, synthetic construct
  SITE 1: note = Residue attached to another sequence via a Lysine 2x polyethylene glycol linker
FGFGRGRQ

SEQ ID NO: 294 - protein, 5 aa, synthetic construct
  SITE 1: note = Acetylated residue
  SITE 5: note = Residue attached to another sequence via a Lysine 2x polyethylene glycol linker
KKKRK

SEQ ID NO: 295 - protein, 8 aa, synthetic construct
  SITE 1: note = Residue attached to another sequence via a Lysine 2x polyethylene glycol linker
FGFGRGRQ

SEQ ID NO: 296 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 297 - protein, 8 aa, synthetic construct
  SITE 1: note = Residue attached to another sequence via a Lysine 2x polyethylene glycol linker
  SITE 3: note = 3-(2-naphthyl)-alanine
FFXGRGRQ

SEQ ID NO: 298 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 299 - protein, 8 aa, synthetic construct
  SITE 1: note = Residue attached to another sequence via a Lysine 2x polyethylene glycol linker
  SITE 3: note = 3-(2-naphthyl)-alanine
FFXGRGRQ

SEQ ID NO: 300 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 301 - protein, 8 aa, synthetic construct
  SITE 1: note = Beta-homophenylalanine attached to another sequence via a Lysine 2x polyethylene glycol linker
  SITE 3: note = 3-(2-naphthyl)-alanine
XFXSRSRQ

SEQ ID NO: 302 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 303 - protein, 8 aa, synthetic construct
  SITE 1: note = Beta-homophenylalanine attached to another sequence via a Lysine 2x polyethylene glycol linker
  SITE 3: note = 3-(2-naphthyl)-alanine
XFXSRSRQ

SEQ ID NO: 304 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 305 - protein, 8 aa, synthetic construct
  SITE 1: note = Beta-homophenylalanine attached to another sequence via a Lysine 2x polyethylene glycol linker
  SITE 3: note = 3-(2-naphthyl)-alanine
XFXGRGRQ

SEQ ID NO: 306 - protein, 7 aa, synthetic construct
  SITE 1: note = Acetylated residue
  SITE 7: note = Residue attached to another sequence via a Lysine 2x polyethylene glycol linker
PKKKRKV

SEQ ID NO: 307 - protein, 8 aa, synthetic construct
  SITE 1: note = Beta-homophenylalanine attached to another sequence via a Lysine 2x polyethylene glycol linker
  SITE 3: note = 3-(2-naphthyl)-alanine
XFXGRGRQ

SEQ ID NO: 308 - protein, 22 aa, synthetic construct
  SITE 1: note = Acetylated residue
  SITE 2: note = Aminohexanoic acid
  SITE 5: note = Beta-alanine
  SITE 8: note = Aminohexanoic acid
  SITE 16: note = Aminohexanoic acid
  SITE 18: note = Beta-alanine
  SITE 20: note = Aminohexanoic acid
  SITE 22: note = Beta-alanine
RXRRXRRXRYQFLIRXRXRXRX

SEQ ID NO: 309 - protein, 8 aa, synthetic construct
  SITE 1: note = Acetylated residue
  SITE 8: note = Residue attached to another sequence via the side chain
PKKKRKVK

SEQ ID NO: 310 - protein, 8 aa, synthetic construct
  SITE 1: note = Residue attached to another sequence via the side chain
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 311 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 312 - protein, 8 aa, synthetic construct
  SITE 1: note = Residue attached to another sequence via a Lysine 2-[2-[2-aminoethoxy]ethoxy]acetic acid linker
FGFGRGRQ

SEQ ID NO: 313 - DNA, 25 bp, synthetic construct
  source 1..25: mol_type = other DNA
atattgctattaccttaacccagaa

SEQ ID NO: 314 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 315 - protein, 8 aa, synthetic construct
  SITE 1: note = Residue attached to another sequence via a Lysine 2-[2-[2-aminoethoxy]ethoxy]acetic acid linker
  SITE 2: note = D-amino acid
  SITE 5: note = D-amino acid
  SITE 7: note = D-amino acid
GFFGRGRQ

SEQ ID NO: 316 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 317 - protein, 8 aa, synthetic construct
  SITE 1: note = Residue attached to another sequence via a Lysine 2-[2-[2-aminoethoxy]ethoxy]acetic acid linker
  SITE 2: note = D-amino acid
FFFGRGRQ

SEQ ID NO: 318 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 319 - protein, 8 aa, synthetic construct
  SITE 1: note = Residue attached to another sequence via a Lysine 2-[2-[2-aminoethoxy]ethoxy]acetic acid linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 320 - protein, 7 aa, synthetic construct
  SITE 1: note = Acetylated residue
  SITE 7: note = Residue attached to another sequence via a Lysine 2-[2-[2-aminoethoxy]ethoxy]acetic acid linker
PKKKRKV

SEQ ID NO: 321 - protein, 8 aa, synthetic construct
  SITE 1: note = Residue attached to another sequence via a Lysine 2-[2-[2-aminoethoxy]ethoxy]acetic acid linker
FGFRRRRQ

SEQ ID NO: 322 - protein, 8 aa, synthetic construct
  VARIANT 1: note = Any amino acid
  VARIANT 6: note = The number of carbons in the side chain may vary
  VARIANT 8: note = The number of carbons in the side chain may vary
XFGFGRGR

SEQ ID NO: 323 - protein, 8 aa, synthetic construct
  VARIANT 1: note = Any amino acid
  SITE 3: note = D-amino acid
  VARIANT 6: note = The number of carbons in the D-arginine side chain may vary
  VARIANT 8: note = The number of carbons in the D-arginine side chain may vary
XGFFGRGR

SEQ ID NO: 324 - protein, 8 aa, synthetic construct
  VARIANT 1: note = Any amino acid
  SITE 3: note = D-amino acid
  VARIANT 6: note = The number of carbons in the side chain may vary
  VARIANT 8: note = The number of carbons in the side chain may vary
XFFFGRGR

SEQ ID NO: 325 - protein, 8 aa, synthetic construct
  VARIANT 1: note = Any amino acid
  SITE 3: note = D-amino acid
  SITE 4: note = 3-(2-naphthyl)-alanine
  SITE 6: note = D-amino acid
  SITE 7: note = D-amino acid
  SITE 8: note = D-amino acid
XFFXGRGR

SEQ ID NO: 326 - protein, 8 aa, synthetic construct
  VARIANT 1: note = Any amino acid
XFGFGRRR

SEQ ID NO: 327 - protein, 8 aa, synthetic construct
  VARIANT 1: note = Any amino acid
XFGFRRRR

SEQ ID NO: 328 - protein, 11 aa, synthetic construct
  VARIANT 1: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = The number of carbons in the side chain may vary
  VARIANT 10: note = Any amino acid
  VARIANT 11: note = The number of carbons in the side chain may vary
  VARIANT 1..11: note = May have D or L stereochemistry
  VARIANT 5..8: note = This region may encompass 1-4 residues
XFFFXXXXRXR

SEQ ID NO: 329 - protein, 11 aa, synthetic construct
  VARIANT 1: note = Any amino acid
  SITE 4: note = 3-(2-naphthyl)-alanine
  VARIANT 5: note = Any amino acid
  VARIANT 6: note = Any amino acid
  VARIANT 7: note = Any amino acid
  VARIANT 8: note = Any amino acid
  VARIANT 9: note = The number of carbons in the side chain may vary
  VARIANT 10: note = Any amino acid
  VARIANT 11: note = The number of carbons in the side chain may vary
  VARIANT 1..11: note = May have D or L stereochemistry
  VARIANT 5..8: note = This region may encompass 1-4 residues
XFFXXXXXRXR

SEQ ID NO: 330 - protein, 8 aa, synthetic construct
  VARIANT 1: note = Any amino acid
  VARIANT 3: note = Any amino acid
  VARIANT 5: note = Any amino acid
  VARIANT 7: note = Any amino acid
  VARIANT 1..8: note = May have D or L stereochemistry
XFXFXRXR

SEQ ID NO: 331 - protein, 8 aa, synthetic construct
  VARIANT 1: note = Any amino acid
  SITE 2: note = Beta-homophenylalanine
  SITE 4: note = 3-(2-naphthyl)-alanine
  VARIANT 1..8: note = May have D or L stereochemistry
XXFXGRGR

SEQ ID NO: 332 - protein, 8 aa, synthetic construct
  VARIANT 1: note = Any amino acid
  SITE 2: note = Beta-homophenylalanine
  SITE 4: note = 3-(2-naphthyl)-alanine
  VARIANT 1..8: note = May have D or L stereochemistry
XXFXSRSR